(19) United States
(12) Patent Application Publication
(10) Pub. No.: US 2025/0333421 A1
(43) Pub. Date: Oct. 30, 2025

(54) PYRIDAZINES OR 1,2,4-TRIAZINES SUBSTITUTED BY SPIROCYCLIC AMINES

(71) Applicant: Janssen Pharmaceutica NV, Beerse (BE)

(72) Inventors: Wei CAI, Shanghai (CN); Xuedong DAI, Shanghai (CN); Olivier Alexis Georges QUEROLLE, Saint Vigor (FR); Johannes Wilhelmus J. THURING, Antwerpen (BE); Xiangjun DENG, Shanghai (CN); Lichao FANG, Shanghai (CN); Liqiang FU, Shanghai (CN); Ming LI, Shanghai (CN); Lianzhu LIU, Shanghai (CN); Yingtao LIU, Shanghai (CN); Yanping XU, Noblesville, IN (US); Vineet PANDE, Vosselaar (BE)

(21) Appl. No.: 18/566,249

(22) PCT Filed: Jun. 2, 2022
(86) PCT No.: PCT/CN2022/096734

§ 371 Date: Dec. 1, 2023

(30) Foreign Application Priority Data
Jun. 3, 2021 (WO) PCT/CN2021/098067

(51) Int. Cl. C07D 487/10; A61K 31/53; A61P 35/02

(52) U.S. Cl. CPC C07D 487/10; A61K 31/53; A61P 35/02

(57) ABSTRACT

The present invention relates to pharmaceutical agents useful for therapy and/or prophylaxis in a mammal, pharmaceutical composition comprising such compounds, and their use as menin/MLL protein/protein interaction inhibitors, useful for treating diseases such as cancer, including but not limited to leukemia, myelodysplastic syndrome (MDS), and myeloproiiferative neoplasms (MPN); and diabetes.

### FIELD OF THE INVENTION

The present invention relates to pharmaceutical agents useful for therapy and/or prophylaxis in a mammal, pharmaceutical composition comprising such compounds, and their use as rnenin/MLL protein/protein interaction inhibitors, useful for treating diseases such as cancer, including but not limited to leukemia, myelodysplastic syndrome (MDS), and myeloproliferative neoplasms (MPN); and diabetes.

### SEQUENCE LISTING

The instant application contains a Sequence Listing which has been submitted electronically in ASCII format and is hereby incorporated by reference in its entirety. Said ASCII copy, created on Sep. 16, 2024, is named JAB7093USPCT2_SL.txt and is 6,020 bytes in size.

### BACKGROUND OF THE INVENTION

Chromosomal rearrangements affecting the mixed lineage leukemia gene (MLL; LL1; KMT2A) result in aggressive acute leukemias across all age groups and still represent mostly incurable diseases emphasizing the urgent need for novel therapeutic approaches. Acute leukemias harboring these chromosomal translocations of MLL represent as lymphoid, myeloid or biphenotypic disease and constitute 5 to 10% of acute leukemias in adults and approximately 70% in infants (Marschalek, Br J Haematol 2011. 152(2), 141-54; Tomizawa et al., Pediatr Blood Cancer 2007. 49(2), 127-32).

MLL is a histone methyltransferase that methylates histone H3 on lysine 4 (H3K4) and functions in multiprotein complexes. Use of inducible loss-of-function alleles of Mll1 demonstrated that Mll1 plays an essential role in sustaining hematopoietic stem cells (HSCs) and developing B cells although its histone methyltransferase activity is dispensable for hematopoiesis (Mishra et al., Cell Rep 2014. 7(4), 1239-47).

Fusion of MLL with more than 60 different partners has been reported to date and has been associated with leukemia formation/progression (Meyer et al., Leukemia 2013. 27, 2165-2176). Interestingly, the SET (Su(var)3-9, enhancer of zeste, and trithorax) domain of MLL is not retained in chimeric proteins but is replaced by the fusion partner (Thiel et al., Bioessays 2012. 34, 771-80). Recruitment of chromatin modifying enzymes like Dot1L and/or the pTEFb complex by the fusion partner leads to enhanced transcription and transcriptional elongation of MLL target genes including HOXA genes (e.g. HOX49) and the HOX cofactor MELS1 as the most prominent ones. Aberrant expression of these genes in turn blocks hematopoietic differentiation and enhances proliferation.

Menin which is encoded by the Multiple Endocrine Neoplasia type 1 (MEN1) gene is expressed ubiquitously and is predominantly localized in the nucleus. It has been shown to interact with numerous proteins and is, therefore, involved in a variety of cellular processes. The best understood function of menin is its role as an oncogenic cofactor of MLL fusion proteins. Menin interacts with two motifs within the N-terminal fragment of MLL that is retained in all fusion proteins, MBM1 (menin-binding motif 1) and MBM2 (Thiel et al., Bioessays 2012. 34, 771-80). Menin/MLL interaction leads to the formation of a new interaction surface for lens epithelium-derived growth factor (LEDGF). Although MLL directly binds to LEDGF, menin is obligatory for the stable interaction between MLL and LEDGF and the gene specific chromatin recruitment of the MLL complex via the PWWP domain of LEDGF (Cermakova et al., Cancer Res 2014. 15, 5139-51; Yokoyama & Cleary, Cancer Cell 2008. 8, 36-46). Furthermore, numerous genetic studies have shown that menin is strictly required for oncogenic transformation by MLL fusion proteins suggesting the menin/MLL interaction as an attractive therapeutic target. For example, conditional deletion of Men1 prevents leukomogenesis in bone marrow progenitor cells ectopically expressing MLL fusions (Chen et al., Proc Natl Acad Sci 2006. 103, 1018-23). Similarly, genetic disruption of menin/MLL fusion interaction by loss-of-function mutations abrogates the oncogenic properties of the MLL fusion proteins, blocks the development of leukemia in vivo and releases the differentiation block of MLL-transformed leukemic blasts. These studies also showed that menin is required for the maintenance of HOX gene expression by MLL fusion proteins (Yokoyama et al., Cell 2005. 123, 207-18). In addition, small molecule inhibitors of menin/MLL interaction have been developed suggesting druggability of this protein/protein interaction and have also demonstrated efficacy in preclinical models of AML (Borkin et al., Cancer Cell 2015. 27, 589-602; Cierpicki and Grembecka, Future Med Chem 2014. 6, 447-462). Together with the observation that menin is not a requisite cofactor of MLL1 during normal hematopoiesis (Li et al., Blood 2013. 122, 2039-2046), these data validate the disruption of menin/MLL interaction as a promising new therapeutic approach for the treatment of MLL rearranged leukemia and other cancers with an active HOX/ME1S1 gene signature. For example, an internal partial tandem duplication (PTD) within the 5′ region of the MLL gene represents another major aberration that is found predominantly in de novo and secondary AML as well as myeloid dysplasia syndromes. Although the molecular mechanism and the biological function of MLL-PTD is not well understood, new therapeutic targeting strategies affecting the menin/MLL interaction might also prove effective in the treatment of MLL-PTD-related leukemias. Furthermore, castration-resistant prostate cancer has been shown to be dependent on the menin/MLL interaction (Malik et al., Nat Med 2015. 21, 344-52).

MLL protein is also known as Histone-lysine N-methyltransferase 2A (KMT2A) protein in the scientific field (UniProt Accession #Q03164).

Several references describe inhibitors targeting the menin-MLL interaction: WO2011029054, J Med Chem 2016, 59, 892-913 describe the preparation of thienopyrimidine and benzodiazepine derivatives; WO2014164543 describes thienopyrimidine and thienopyridine derivatives; Nature Chemnical Biology March 2012, 8, 277-284 and Ren, J.; et al. Bioorg Med Chem Lett (2016), 26(18), 4472-4476 describe thienopyrimidine derivatives; J Med Chem 2014, 77, 1543-1556 describes hydroxy- and aminomethylpiperidine derivatives; Future Med Chem 2014, 6, 447-462 reviews small molecule and peptidomimetic compounds; WO2016195776 describes furo[2,3-d]pyrimidine, 9H-purine, [1,3]oxazolo[5,4-dlpyrimidine, [1,3]oxazolo[4,5-d]pyrimidine, [1,3]thiazolo[5,4-dlpyrimidine, thieno[2,3-b]pyridine and thieno[2,3-d]pyrimidine derivatives: WO2016197027 describes 5,6,7,8-tetrahydropyrido[3,4-d]pyrimidine, 5,6,7,8-tetrahydropyrido]4,3-d]pyrimidine, pyrido[2,3-d]pyrimidine and quinoline derivatives; and WO2016040330 describes thienopyrimidine and thienopyridine compounds. WO2017192543 describes piperidines as Menin inhibitors. WO2017112768, WO2017207387, WO2017214367, WO2018053267 and WO2018024602 describe inhibitors of the menin-MLL interaction. WO2017161002 and WO2017161028 describe inhibitors of menin-MLL. WO2018050686, WO2018050684 and WO2018109088 describe inhibitors of the menin-MLL interaction. WO2018226976 describes methods and compositions for inhibiting the interaction of menin with MLL proteins. WO2018175746 provides methods of treatment for hematological malignancies and Ewing's sarcoma. WO2018106818 and WO2018106820 provide methods of promoting proliferation of a pancreatic cell. WO2018153312 discloses azaspiro compounds relating to the field of medicinal chemistry. WO2017132398 discloses methods comprising contacting a leukemia cell exhibiting an NPM1 mutation with a pharmacologic inhibitor of interaction between MLL and Menin. WO2019060365 describes substituted inhibitors of menin-MLL. WO2020069027 describes the treatment of hematological malignancies with inhibitors of menin. Krivtsov et al., Cancer Cell 2019. No. 6 Vol. 36, 660-673 describes a menin-MLL inhibitor. WO2021121327 describes substituted straight chain spiro derivatives and their use as menin/MLL protein/protein interaction inhibitors.

### DESCRIPTION OF THE INVENTION

The present invention concerns novel compounds of Formula (I),

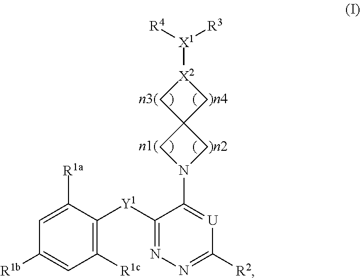

and the tautomers and the stereoisomeric forms thereof, wherein
R^{1a} represents —C(═O)—NR^{xa}R^{xb}; Het; or

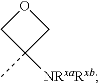

Het represents a 5- or 6-membered monocyclic aromatic ring containing one, two or three nitrogen atoms and optionally a carbonyl moiety;
wherein said 5- or 6-membered monocyclic aromatic ring is optionally substituted with one, two or three substituents selected from the group consisting of C₃₋₆cycloalkyl and C₁₋₄alkyl;
R^{xa} and R^{xb} are each independently selected from the group consisting of hydrogen; C₁₋₄alkyl; C₃₋₆cycloalkyl; C₁₋₄alkyl substituted with 1, 2 or 3 halo atoms; and C₁₋₄alkyl substituted with one —OH, —OC₁₋₄alkyl, or NR^{11c}R^{11d};
R^{1b} represents F or Cl;
R^{1c} represents H or halo;
Y¹ represents —CR^{5a}R^{5b}—, —O— or —NR^{5c}—.
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents C₁₋₅alkyl;

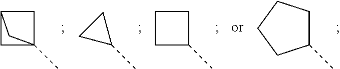

R^{5a}, R^{5b}, R^{5c}, R^{7a}, and R^{7b}, are each independently selected from the group consisting of hydrogen, C₁₋₄alkyl and C₃₋₆cycloalkyl;
R³ represents —C₁₋₆alkyl-NR^{8a}R^{8b}, —C₁₋₆alkyl-C(═O)—NR^{9a}R^{9b}, —C₁₋₆alkyl-OH, or
C₁₋₆alkyl-NR¹¹—C(═O)—O—C₁₋₄alkyl-O—C(═O)—C₁₋₄alkyl;
wherein each of the C₁₋₄alkyl or C₁₋₆alkyl moieties in the R³ definitions independently of each other may be substituted with one, two or three substituents each independently selected from the group consisting of cyano, halo, —OH, and —O—C₁₋₄alkyl;
R^{8a} and R^{8b} are each independently selected from the group consisting of hydrogen;
C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of —OH, cyano, halo, —C—C, —CH═CH, —S—C₁₋₄alkyl, —S(═O)₂—C₁₋₄alkyl, —S(═O)₂—NR^{11a}R^{11b}, —O—C₁₋₄alkyl, —C(═O)—NR^{10a}R^{10c}R^{10b}, —NR^{10c}—C(═O)—C₁₋₄alkyl, —O—C₁₋₄alkyl substituted with one NR^{11a}R^{11b} and —O—C₁₋₄alkyl substituted with one, two or three halo atoms;
R^{9a}, R^{9b}, R^{10a}, R^{10b}, R^{10c}, R¹¹, R^{11a}, R^{11b}, R^{12a}, and R^{12b} are each independently selected from the group consisting of hydrogen and C₁₋₆alkyl;
R^{11c} and R^{11d} are each independently selected from the group consisting of hydrogen, C₁₋₆alkyl, and —C(═O)—C₁₋₄alkyl;
and the pharmaceutically acceptable salts and the solvates thereof;
provided however that at least one of the following conditions is fulfilled:
   a) R^{1a} represents Het wherein the 5- or 6-membered monocyclic aromatic ring is substituted with three substituents selected from the group consisting of C₃₋₆cycloalkyl and C₁₋₄alkyl;
   b) R^{1a} represents —C(═O)—NR^{xa}R^{xb}; and R^{xa} is selected from the group consisting of C₁₋₄alkyl substituted with one —OH, —OC₁₋₄alkyl, or NR^{11c}R^{11d}; and C₁₋₄alkyl substituted with 1, 2 or 3 halo atoms;
   c) R¹⁰ represents halo;
   d) R⁴ is other than isopropyl;
   e) R³ represents —C₁₋₆alkyl-NR^{8a}R^{8b}; and R^{8a} is C₁₋₆alkyl substituted with one, two or three substituents each independently selected from the group consisting of —C—C, —CH═CH, —S—C₁₋₄alkyl, —S(═O)₂—NR^{11a}R^{11b}—O—C₁₋₄alkyl substituted with one NR^{11a}R^{11b} and —O—C₁₋₄alkyl substituted with one, two or three halo atoms.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer, including but not limited to leukemia, myelodysplastic syndrome (MDS), and myeloproliferative neoplasms (MPN); and diabetes.

In a particular embodiment, the invention relates to a compound of Formula (I), a pharmaceutically acceptable salt, or a solvate thereof, for use in the treatment or in the prevention of cancer.

In a specific embodiment said cancer is selected from leukemias, lymphomas, myelomas or solid tumor cancers (e.g. prostate cancer, lung cancer, breast cancer, pancreatic cancer, colon cancer, liver cancer, melanoma and glioblastoma, etc.). In some embodiments, the leukemias include acute leukemias, chronic leukemias, myeloid leukemias, myelogeneous leukemias, lymphoblastic leukemias, lymphocytic leukemias. Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, leukemias exhibiting HOX/MEIS1 gene expression signatures etc.

In particular, compounds according to the present invention and the pharmaceutical compositions thereof may be useful in the treatment or prevention of leukemias, in particular nucleophosmin (NPMI)-mutated leukemias, e.g. NPM1c.

In an embodiment, compounds of Formula (I) and the pharmaceutically acceptable salts, and the solvates thereof, may have improved metabolic stability properties.

In an embodiment, compounds of Formula (I) and the pharmaceutically acceptable salts, and the solvates thereof, may have extended in vivo half-life (T1/2).

In an embodiment, compounds of Formula (I) and the pharmaceutically acceptable salts, and the solvates thereof, may have improved oral bioavailability.

In an embodiment, compounds of Formula (I) and the pharmaceutically acceptable salts, and the solvates thereof, may reduce tumor growth e.g., tumours harbouring MLL (KMT2A) gene rearrangements/alterations and/or NPM1 mutations.

In an embodiment, compounds of Formula (I) and the pharmaceutically acceptable salts, and the solvates thereof, may have improved PD properties in vivo during a prolonged period of time, e.g. inhibition of target gene expression such as MEIS1 and upregulation of differentiation marker over a period of at least 16 hours.

In an embodiment, compounds of Formula (I) and the pharmaceutically acceptable salts, and the solvates thereof, may have an improved safety profile (e.g. reduced hERG inhibition; improved cardiovascular safety).

In an embodiment, compounds of Formula (I) and the pharmaceutically acceptable salts, and the solvates thereof, may be suitable for Q.D. dosing (once daily).

The invention also relates to the use of a compound of Formula (I), a pharmaceutically acceptable salt, or a solvate thereof, in combination with an additional pharmaceutical agent for use in the treatment or prevention of cancer, including but not limited to leukemia, myelodysplastic syndrome (MDS), and myeloproliferative neoplasms (MPN); and diabetes.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer, including but not limited to leukemia, myelodysplastic syndrome (MDS), and myeloproliferative neoplasms (MPN); and diabetes.

Additionally, the invention relates to a method of treating or preventing a cell proliferative disease in a warm-blooded animal which comprises administering to the said animal an effective amount of a compound of Formula (I), a pharmaceutically acceptable salt, or a solvate thereof, as defined herein, or a pharmaceutical composition or combination as defined herein.

### DETAILED DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain saturated hydrocarbon radical having from 1 to 4 carbon atoms, such as methyl, ethyl, n-propyl, isopropyl, n-butyl, s-butyl, t-butyl and the like.

Similar, the term ‘C₁₋₆alkyl’ as used herein as a group or part of a group represents a straight or branched chain saturated hydrocarbon radical having from 1 to 6 carbon atoms, such as methyl, ethyl, n-propyl, isopropyl, n-butyl, s-butyl, t-butyl, n-pentyl, n-hexyl and the like.

The term ‘C₃₋₆cycloalkyl’ as used herein as a group or part of a group defines a saturated, cyclic hydrocarbon radical having from 3 to 6 carbon atoms, such as cyclopropyl, cyclobutyl, cyclopentyl and cyclohexyl.

It will be clear for the skilled person that S(═O)₂ or SO₂ represents a sulfonyl moiety.

It will be clear for the skilled person that CO or C(═O) represents a carbonyl moiety.

It will be clear for the skilled person that a group such as —CRR— represents

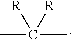

An example of such a group is —CR^{5a}R^{5b}—.

It will be clear for the skilled person that a group such as —NR— represents

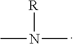

An example of such a group is —NR^{5c}—.

Non-limiting examples of ‘monocyclic 5- or 6-membered aromatic rings containing one, two or three nitrogen atoms and optionally a carbonyl moiety’, include, but are not limited to pyrazolyl, imidazolyl, pyridinyl, pyridazinyl, pyrimidinyl, pyrazinyl, triazinyl or 1,2-dihydro-2-oxo-4-pyridinyl.

The skilled person will understand that a 5- or 6-membered monocyclic aromatic ring containing one, two or three nitrogen atoms and a carbonyl moiety includes, but is not limited to

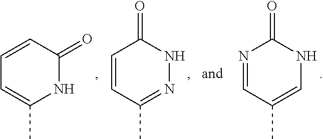

When any variable occurs more than one time in any constituent, each definition is independent.

When any variable occurs more than one time in any formula (e.g. Formula (I)), each definition is independent.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture (isolation after a reaction e.g. purification by silica gel chromatography). In a particular embodiment, when the number of substituents is not explicitly specified, the number of substituents is one.

Combinations of substituents and/or variables are permissible only if such combinations result in chemically stable compounds. ‘Stable compound’ is in this context meant to indicate a compound that is sufficiently robust to survive isolation to a useful degree of purity from a reaction mixture (isolation after a reaction e.g. purification by silica gel chromatography).

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

Within the context of this invention ‘saturated’ means ‘fully saturated’, if not otherwise specified.

Unless otherwise specified or clear from the context, aromatic rings groups, can be attached to the remainder of the molecule of Formula (I) through any available ring carbon atom (C-linked) or nitrogen atom (N-linked).

Unless otherwise specified or clear from the context, aromatic rings groups, may optionally be substituted, where possible, on carbon and/or nitrogen atoms according to the embodiments.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof. The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration: for example if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (?), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer.

Some of the compounds according to Formula (I) may also exist in their tautomeric form. Such forms in so far as they may exist, although not explicitly indicated in the above Formula (I) are intended to be included within the scope of the present invention. It follows that a single compound may exist in both stereoisomeric and tautorneric form.

Pharmaceutically acceptable salts include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I) and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term “prodrug” includes any compound that, following oral or parenteral administration, in particular oral administration, is metabolised in vivo to a (more) active form in an experimentally-detectable amount, and within a predetermined time (e.g. within a dosing interval of between 0.5 and 24 hours, or e.g. within a dosing interval of between 6 and 24 hours (i.e. once to four times daily)). For the avoidance of doubt, the term “parenteral” administration includes all forms of administration other than oral administration, in particular intravenous (IV), intramuscular (IM), and subcutaneous (SC) injection.

Prodrugs may be prepared by modifying functional groups present on a compound in such a way that the modifications are cleaved in vivo when such prodrug is administered to a mammalian subject. The modifications typically are achieved by synthesising the parent compound with a prodrug substituent. In general, prodrugs include compounds wherein a hydroxyl, amino, sulthydryl, carboxy or carbonyl group is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

The term solvate comprises the solvent addition forms as well as the salts thereof which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form.

Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C, ¹³C and ¹⁸F. Preferably, the isotope is selected from the group of ²H, ³H, ¹¹C and ¹⁸F. More preferably, the isotope is 2H, ³H or ¹³C. More preferably, the isotope is ²H or ¹³C. More preferably, the isotope is ²H. In particular, deuterated compounds and ¹³C-enriched compounds are intended to be included within the scope of the present invention. In particular, deuterated compounds are intended to be included within the scope of the present invention.

Certain isotopically-labeled compounds of the present invention (e.g., those labeled with ³H and ¹⁴C) may be useful for example in substrate tissue distribution assays. Tritiated (3H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

The present invention relates in particular to compounds of Formula (I) as defined herein, and the tautomers and the stereoisomeric forms thereof, wherein
R^{1a} represents —C(═O)—NR^{xa}R^{xb}; Het; or

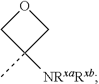

Het represents a 5- or 6-membered monocyclic aromatic ring containing one, two or three nitrogen atoms and optionally a carbonyl moiety;
wherein said 5- or 6-membered monocyclic aromatic ring is optionally substituted with one, two or three substituents selected from the group consisting of C₃₋₆cycloalkyl and C₁₋₄alkyl;
R^{xa} and R^{xb} are each independently selected from the group consisting of hydrogen;
C₁₋₄alkyl; C₃₋₆cycloalkyl; C₁₋₄alkyl substituted with 1, 2 or 3 halo atoms; and C₁₋₄alkyl substituted with one —OH, —OC₁₋₄alkyl, or NR^{11c}R^{11d};
R^{1b} represents F or Cl;
R^{11c} represents H or halo;
Y¹ represents —O—;
R² represents hydrogen;
U represents N;
n1, n2, n3 and n4 are each independently selected from 1 and 2;
X¹ represents CH, and X² represents N;
R⁴ represents C₁₋₅alkyl;

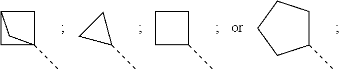

R³ represents —C₁₋₆alkyl-NR^{8a}R^{8b};
wherein the C₁₋₆alkyl moiety in the R³ definition may be substituted with one, two or three substituents each independently selected from the group consisting of cyano, halo, —OH, and —O—C₁₋₄alkyl;
R^{8a} and R^{8b} are each independently selected from the group consisting of hydrogen; C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of —OH, cyano, halo, —C—C, —CH═CH, —S—C₁₋₄alkyl, —S(═O)₂—C₁₋₄alkyl, —S(═O)₂—NR^{11a}R^{11b}, —O—C₁₋₄alkyl, —C(═O)—NR^{10a}R^{10b}, —NR^{10c}—C(═O)—C₁₋₄alkyl, —O—C₁₋₄alkyl substituted with one NR^{11a}R^{11b}, and —O—C₁₋₄alkyl substituted with one, two or three halo atoms;
R^{10a}, R^{10b}, R^{10c}, R^{11a}, R^{11b}, R^{12a}, and R^{12b} are each independently selected from the group consisting of hydrogen and C₁₋₆alkyl;
R^{11c} and R^{11d} are each independently selected from the group consisting of hydrogen, C₁₋₆alkyl, and —C(═O)—C₁₋₄alkyl;
and the pharmaceutically acceptable salts and the solvates thereof;
provided however that at least one of the following conditions is fulfilled:
   a) R^{1a} represents Het wherein the 5- or 6-membered monocyclic aromatic ring is substituted with three substituents selected from the group consisting of C₃₋₆cycloalkyl and C₁₋₄alkyl;
   b) R^{1a} represents —C(═O)—NR^{xa}R^{xb}; and R^{xa} is selected from the group consisting of C₁₋₄alkyl substituted with one —OH, —OC₁₋₄alkyl, or NR^{11c}R^{11d}; and C₁₋₄alkyl substituted with 1, 2 or 3 halo atoms;
   c) R¹⁰ represents halo;
   d) R⁴ is other than isopropyl;
   e) R³ represents —C₁₋₆alkyl-NR^{8a}R^{8b}; and R^{1a} is C₁₋₆alkyl substituted with one, two or three substituents each independently selected from the group consisting of —C—C, —CH═CH, —S—C₁₋₄alkyl, —S(═O)₂—NR^{11a}R¹¹, —O—C₁₋₄alkyl substituted with one NR^{11a}R^{11b} and —O—C₁₋₄alkyl substituted with one, two or three halo atoms.

The present invention relates in particular to compounds of Formula (I) as defined herein, and the tautomers and the stereoisomeric forms thereof, wherein
R^{1a} represents —C(═O)—NR^{xa}R^{xb};
R^{xa} and R^{xb} are each independently selected from the group consisting of C₁₋₄alkyl; and
C₁₋₄alkyl substituted with one —OH, or NR^{11c}R^{11d};
R^{1b} represents F;
R^{1c} represents H or halo;
Y¹ represents —O—;
R² represent hydrogen;
n1, n2, n3 and n4 are each independently selected from 1 and 2;
X¹ represents CH, and X² represents N;
R⁴ represents C₁₋₅alkyl;
R³ represents —C₁₋₆alkyl-NR^{8a}R^{8b};
wherein the C₁₋₆alkyl moiety in the R³ definition may be substituted with one, two or three —OH substituents;
R^{8a} and R^{8b} are each independently selected from the group consisting of hydrogen; C₁₋₆alkyl; and C₁₋₆alkyl substituted with one, two or three substituents each independently selected from the group consisting of —C—C, —CH═CH, —S—C₁₋₄alkyl, —S(═O)₂—NR^{11a}R^{11b}—O—C₁₋₄alkyl, —O—C₁₋₄alkyl substituted with one NR^{11a}R^{11b} and —O—C₁₋₄alkyl substituted with one, two or three halo atoms;
R^{11a} and R^{11b} represent hydrogen;
R^{11c} and R^{11d} are each independently selected from the group consisting of hydrogen and —C(═O)—C₁₋₄alkyl;
and the pharmaceutically acceptable salts and the solvates thereof;
provided however that at least one of the following conditions is fulfilled:
   a) R^{1a} represents —C(═O)—NR^{xa}R^{xb}; and R^{xa} is selected from the group consisting of C₁₋₄alkyl substituted with one —OH or NR^{11c}R^{11d};
   b) R^{1c} represents halo;
   c) R⁴ tert-butyl;
   d) R³ represents —C₁₋₆alkyl-NR^{8a}R^{1b}; and R^{8a} is C₁₋₆alkyl substituted with one, two or three substituents each independently selected from the group consisting of —C≡C, —CH═CH, —S—C₁₋₄alkyl, —S(═O)₂—NR^{11a}R^{11b}, —O—C₁₋₄alkyl substituted with one NR^{11a}R^{11b}, and —O—C₁₋₄alkyl substituted with one, two or three halo atoms.

In an embodiment, the present invention relates to those compounds of Formula (I) and the pharmaceutically acceptable salts, and the solvates thereof, or any subgroup thereof as mentioned in any of the other embodiments, wherein R^{1b} represents F.

In an embodiment, the present invention relates to those compounds of Formula (I) and the pharmaceutically acceptable salts, and the solvates thereof, or any subgroup thereof as mentioned in any of the other embodiments, wherein R² represents hydrogen.

In an embodiment, the present invention relates to those compounds of Formula (I) and the pharmaceutically acceptable salts, and the solvates thereof, or any subgroup thereof as mentioned in any of the other embodiments, wherein n1 is 1, n2 is 2, n3 is 1, and n4 is 1.

In an embodiment, the present invention relates to those compounds of Formula (I) and the pharmaceutically acceptable salts, and the solvates thereof, or any subgroup thereof as mentioned in any of the other embodiments, wherein Y¹ represents —O—.

In an embodiment, the present invention relates to those compounds of Formula (I) and the pharmaceutically acceptable salts, and the solvates thereof, or any subgroup thereof as mentioned in any of the other embodiments, wherein
Y¹ represents —O—; and
U represents N.

In an embodiment, the present invention relates to those compounds of Formula (I) and the pharmaceutically acceptable salts, and the solvates thereof, or any subgroup thereof as mentioned in any of the other embodiments, wherein Li represents N.

In an embodiment, the present invention relates to those compounds of Formula (I) and the pharmaceutically acceptable salts, and the solvates thereof, or any subgroup thereof as mentioned in any of the other embodiments, wherein
Y¹ represents —O—;
U represents N;
R^{1b} represents F; and
R² represents hydrogen.

In an embodiment, the present invention relates to those compounds of Formula (I) and the pharmaceutically acceptable salts, and the solvates thereof, or any subgroup thereof as mentioned in any of the other embodiments, wherein
R^{11a} represents —C(═O)—NR^{xa}R^{xb}.
Y¹ represents —O—;
U represents N;
R^{1b} represents F;
R^{1c} represents H;
R² represents hydrogen:
R⁴ represents C₁₋₅alkyl; and
R³ represents —C₁₋₆alkyl-NR^{8a}R^{8b}.

In an embodiment, the present invention relates to those compounds of Formula (I) and the pharmaceutically acceptable salts, and the solvates thereof, or any subgroup thereof as mentioned in any of the other embodiments, wherein
R^{1a} represents —C(═O)—NR^{xa}R^{xb}.
Y¹ represents —O—;
U represents N;
n1 is 1, n2 is 2, n3 is 1, and n4 is 1.
R^{1b} represents F;
R^{1c} represents H;
R² represents hydrogen:
R⁴ represents C₁₋₅alkyl; and
R³ represents —C₁₋₆alkyl-NR^{8a}R^{8b}.

In an embodiment, the present invention relates to those compounds of Formula (I) and the pharmaceutically acceptable salts, and the solvates thereof, or any subgroup thereof as mentioned in any of the other embodiments, wherein R^{1a} represents —C(═O)—NR^{xa}R^{xb}.

In an embodiment, the present invention relates to those compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}.

In an embodiment, the present invention relates to those compounds of Formula (I) and the pharmaceutically acceptable salts, and the solvates thereof, or any subgroup thereof as mentioned in any of the other embodiments, wherein R³ represents —CH₂—CH₂—CH₂—NR^{8a}R^{8b}.

In an embodiment, the present invention relates to those compounds of Formula (I) and the pharmaceutically acceptable salts, and the solvates thereof, or any subgroup thereof as mentioned in any of the other embodiments, wherein C₁₋₆alkyl in the R³ definition —C₁₋₆alkyl-NR^{1a}R^{8b} is limited to —CH₂—CH₂—CH₂—.

In an embodiment, the present invention relates to those compounds of Formula (I) and the pharmaceutically acceptable salts, and the solvates thereof, or any subgroup thereof as mentioned in any of the other embodiments, wherein at least one of the following conditions is fulfilled:
a) R^{1a} represents —C(═O)—NR^{xa}R^{xb}; and RY is selected from the group consisting of C₁₋₄alkyl substituted with one —OH, —OC₁₋₄alkyl, or NR^{11c}R^{11c}; and C₁₋₄alkyl substituted with 1, 2 or 3 halo atoms;
b) R^{1c} represents halo;
c) R⁴ is other than isopropyl;
d) R³ represents —C₁₋₆alkyl-NR^{8a}R^{8b}; and R^{8a} is C₁₋₆alkyl substituted with one, two or three substituents each independently selected from the group consisting of —C≡C, —CH═CH, —S—C₁₋₄alkyl, —S(═O)₂—NR^{11a}R^{11b}, —O—C₁₋₄alkyl substituted with one NR^{11a}R^{11a}, and —O—C₁₋₄alkyl substituted with one, two or three halo atoms.

In an embodiment, the present invention relates to those compounds of Formula (I) and the pharmaceutically acceptable salts, and the solvates thereof, or any subgroup thereof as mentioned in any of the other embodiments, wherein R^{1a} represents Het wherein the 5- or 6-membered monocyclic aromatic ring is substituted with three substituents selected from the group consisting of C₃₋₆cycloalkyl and C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R^{a} represents —C(═O)—NR^{xa}R^{xb}; and R^{xa} is selected from the group consisting of C₁₋₄alkyl substituted with one —OH, —OC₁₋₄alkyl, or NR^{11c}R^{11d}; and C₁₋₄alkyl substituted with 1, 2 or 3 halo atoms.

In an embodiment, the present invention relates to those compounds of Formula (I) and the pharmaceutically acceptable salts, and the solvates thereof, or any subgroup thereof as mentioned in any of the other embodiments, wherein R¹⁰ represents halo.

In an embodiment, the present invention relates to those compounds of Formula (I) and the pharmaceutically acceptable salts, and the solvates thereof, or any subgroup thereof as mentioned in any of the other embodiments, wherein R^{1c} represents Br.

In an embodiment, the present invention relates to those compounds of Formula (I) and the pharmaceutically acceptable salts, and the solvates thereof, or any subgroup thereof as mentioned in any of the other embodiments, wherein R⁴ is other than isopropyl.

In an embodiment, the present invention relates to those compounds of Formula (I) and the pharmaceutically acceptable salts, and the solvates thereof, or any subgroup thereof as mentioned in any of the other embodiments, wherein R⁴ is tert-butyl.

In an embodiment, the present invention relates to those compounds of Formula (I) and the pharmaceutically acceptable salts, and the solvates thereof, or any subgroup thereof as mentioned in any of the other embodiments, wherein R³ represents —C₁₋₆alkyl-NR^{8a}R^{8b}; and R^{8a} is C₁₋₆alkyl substituted with one, two or three substituents each independently selected from the group consisting of —C≡C, —CH═CH, —S—C₁₋₄alkyl, —S(═O)₂—NR^{11a}R^{11b}, —O—C₁₋₄alkyl substituted with one NR^{11a}R^{11b}, and —O—C₁₋₄alkyl substituted with one, two or three halo atoms.

In an embodiment, the present invention relates to a subgroup of Formula (I) as defined in the general reaction schemes.

In an embodiment the compound of Formula (I) is selected from the group consisting of any of the exemplified compounds,

tautomers and stereoisomeric forms thereof,

and the free bases, any pharmaceutically acceptable salts, and the solvates thereof.

All possible combinations of the above indicated embodiments are considered to be embraced within the scope of the invention.

### Methods for the Preparation of Compounds of Formula (I)

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or prepared by standard synthetic processes commonly used by those skilled in the art of organic chemistry. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups (PG) can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

### General Synthetic Schemes

All abbreviations used in the general schemes are as defined in the Table in the part Examples. Variables are as defined in the scope or as specifically defined in the general Schemes.

### Part A) Schemes 1a, 1b, 1c, 2a, 2b and 3

Rn=C₁₋₆alkyl-NR^{8a}PG or C₁₋₆alkyl-OPG or C₁₋₆alkyl-C(═O)OR^{9a}, PG=protecting group

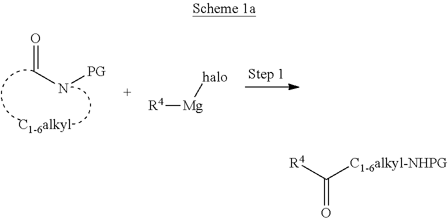

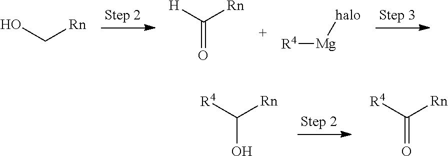

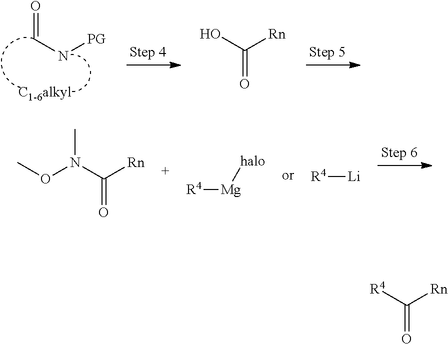

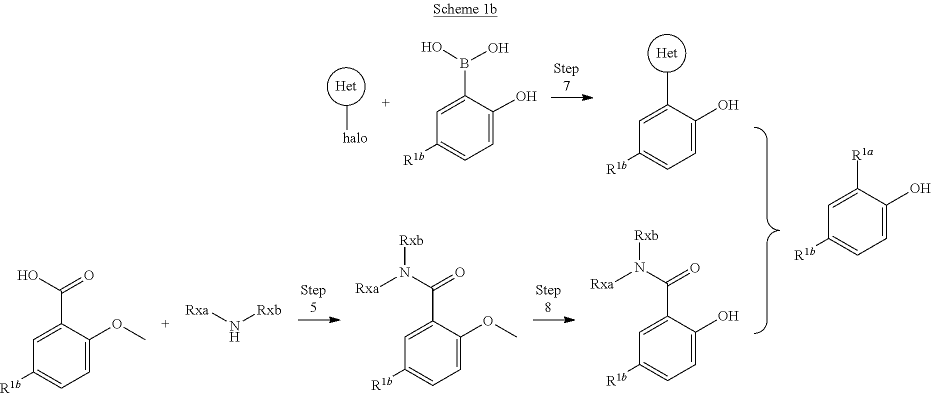

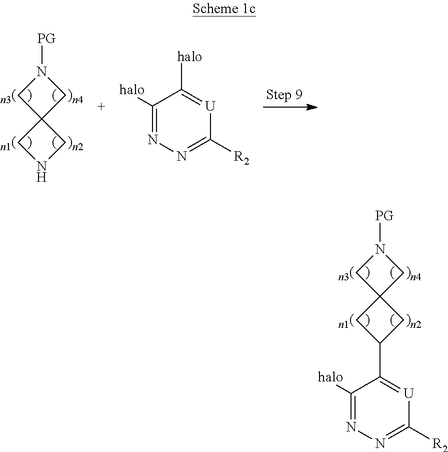

In Scheme 1a, 1b and 1c the following reaction conditions apply:
Step 1: at a suitable temperature such as for example −70° C., in the presence of a suitable base such as for example TMEDA and a suitable organometallic reagent such as for example isopropylmagnesium bromide, in a suitable solvent such as for example THF;
Step 2: at a suitable temperature such as for example from 0° C. to RT, in the presence of a suitable oxidative reagent such as for example DMP, in a suitable solvent such as for example DCM;
Step 3: at a suitable temperature such as for example from −20° C. to RT, in the presence of a suitable organometallic reagent such as for example isopropylmagnesium bromide, in a suitable solvent such as for example THF;
Step 4: at a suitable temperature such as for example 80° C., in the presence of a suitable base such as for example NaOH, in suitable solvents such as for example THF and H₂O;

Step 5: at a suitable temperature such as for example RT, in the presence of suitable amide condensation reagents such as for example EDCI and HOBt, in the presence of a suitable base such as for example NMM, in a suitable solvent such as for example DCM;

Step 6: at a suitable temperature such as for example −70° C., in the presence of a suitable organometallic reagent such as for example isopropyllithium, in a suitable solvent such as for example THF;

Step 7: at a suitable temperature such as for example 90° C., in the presence of a suitable organometallic catalyst such as for example Pd(dppf)Cl₂, in the presence of a suitable base such as for example Na₂CO₃, in suitable solvents such as for example 1,4-dioxane and H₂O;

Step 8: at a suitable temperature such as for example from 0° C. to R T, in the presence of a suitable Lewis acid such as for example BBr₃, in a suitable solvent such as for example DCM;

Step 9: at a suitable temperature such as for example from −78° C. to 40° C., in particular from 0° C. to RT, in the presence of a suitable base such as for example TEA, DBU or K₂CO₃, in a suitable solvent such as for example DCM, THF or DMF;

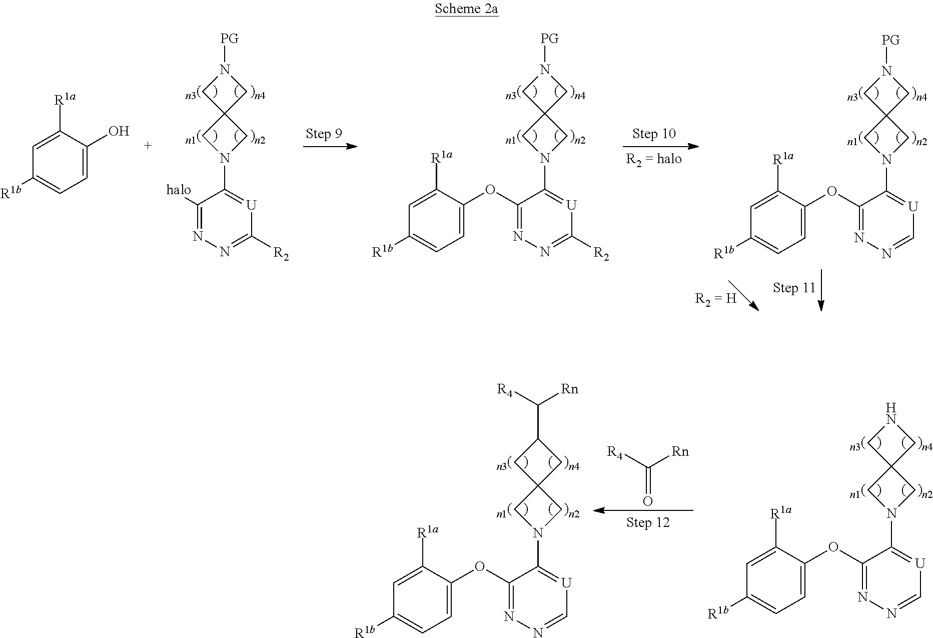

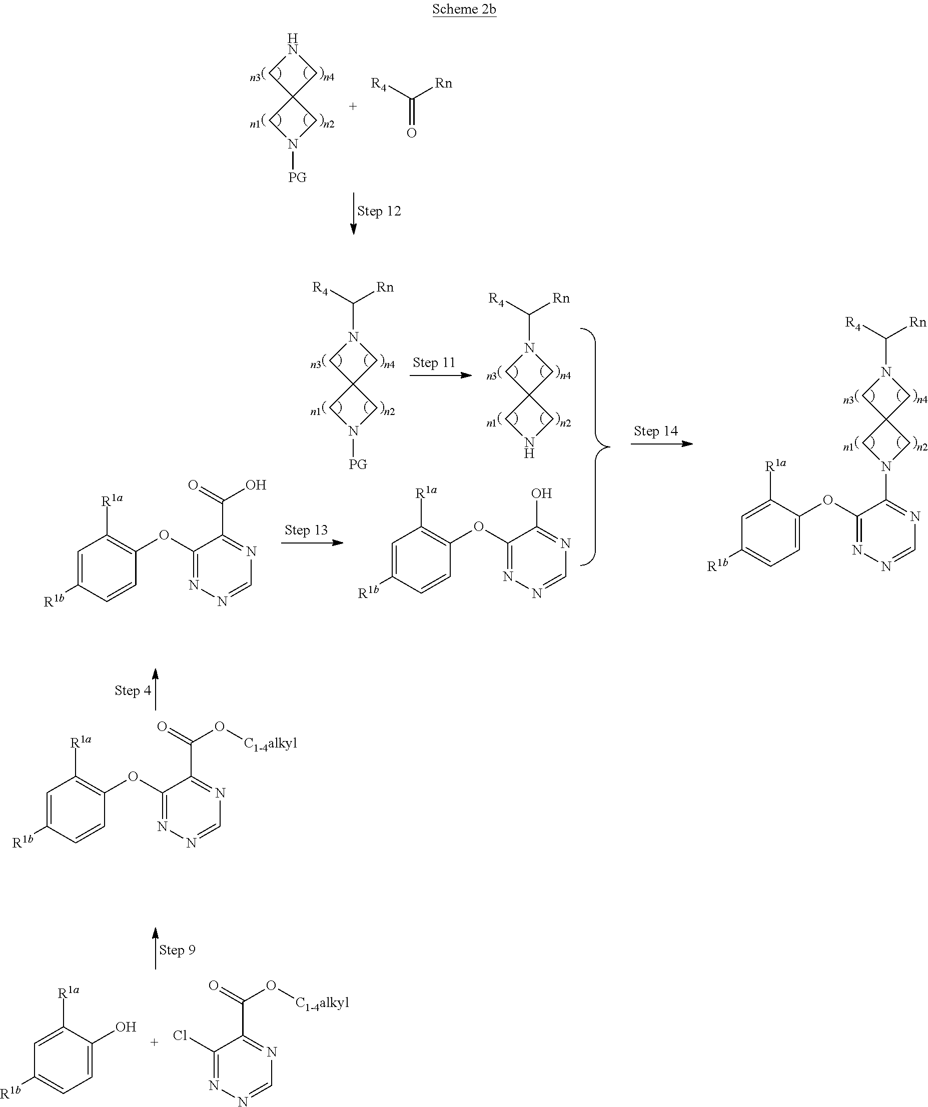

In Scheme 2a and 2b, the following reaction conditions apply:

Step 9: See Step 9 in Scheme 1;

Step 10: at a suitable temperature such as for example RT, in the presence of a suitable catalyst such as for example Pd/C, in the presence of a suitable reductive reagent such as for example H₂, optionally in the presence of a suitable base such as for example TEA, in a suitable solvent such as for example THF;

Alternatively, at a suitable temperature such as RT, in the presence of a suitable catalyst such as for example Pd(dppf)Cl₂-DCM complex, a suitable reducing agent such NaBH₄, a suitable base such as for example TMEDA, in a suitable solvent such as for example THF.

Step 11: for N deprotection, at a suitable temperature such as for example RT, in the presence of a suitable acid as for example TFA, in a suitable solvent such as for example DCM; for O deprotection, at a suitable temperature such as for example RT, in the presence of a suitable acid as for example 4-methylbenzenesulfonic acid, in a suitable solvent such as for example MeOH;

Step 12: at a suitable temperature such as for example 80° C., optionally in the presence of a suitable Lewis acid such as for example ZnCl₂, in the presence of a suitable reductive reagent such as for example NaBH₃CN, in a suitable solvent such as for example MeOH;

Step 13: at a suitable temperature such as for example RT, in the presence of a suitable organometallic catalyst such as for example Ag(Phen)₂OTf, in the presence of a suitable brominating reagent such as for example 1,3-dibromo-1,3,5-triazinane-2,4,6-trione, in a suitable solvent such as for example DCE;

Step 14: at a suitable temperature such as for example RT, in the presence of a suitable chlorinating reagent such as for example oxalyl chloride, in the presence of DMF, in a suitable solvent such as for example DCM.

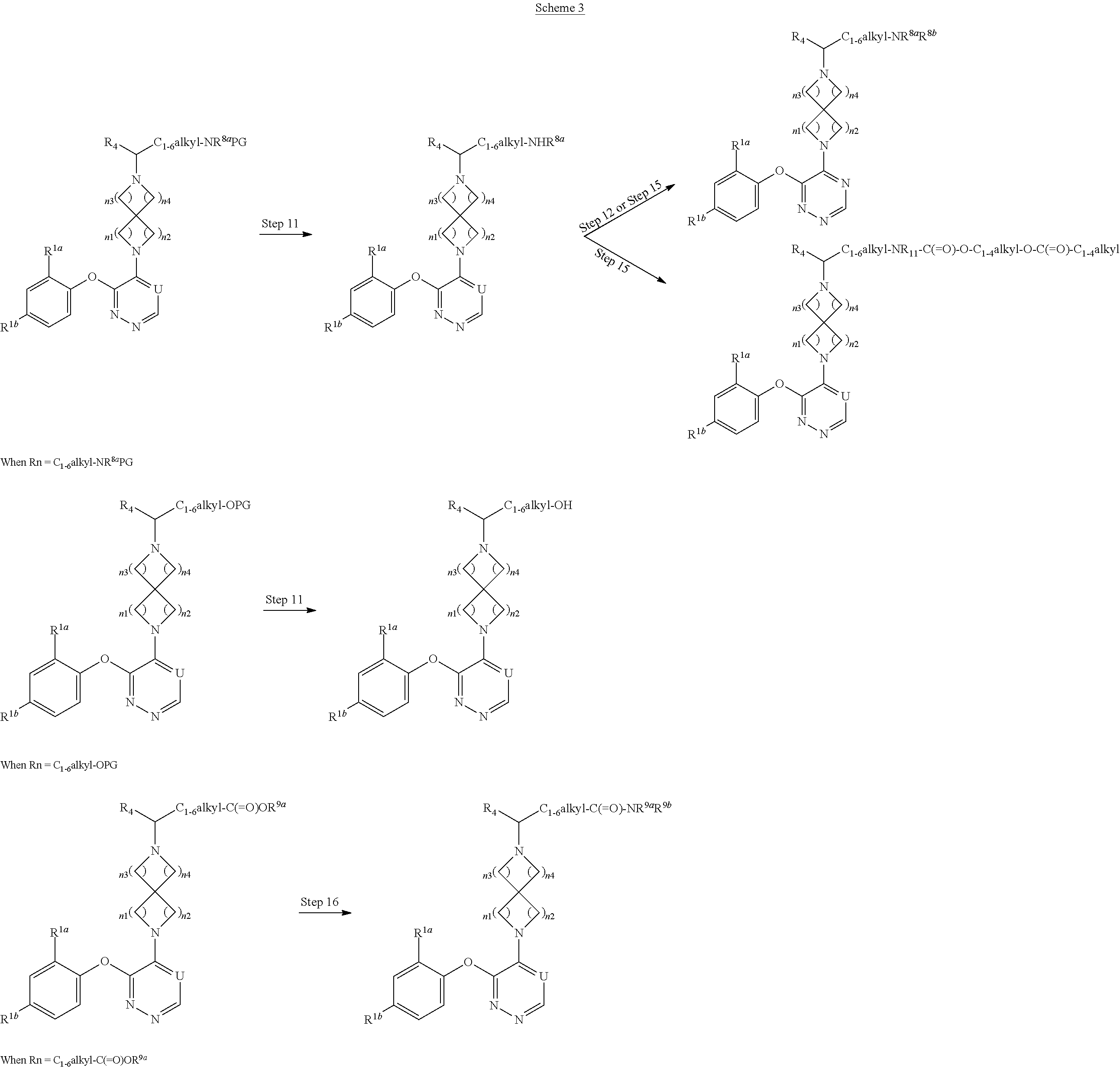

In Scheme 3, the following reaction conditions apply:

Step 11-12: See Step 11-12 in Scheme 2;

Step 15: at a suitable temperature such as for example 80° C., in the presence of a suitable base such as for example Cs₂CO₃, in suitable solvent such as for example DMF;

Step 16: at a suitable temperature such as for example 40° C., in the presence of a suitable base such as for example ammonia, in suitable solvent such as for example 1,4-dioxane.

### Part B) Schemes 4, 5, 6, 7, 8, 9, 10, 11 and 12

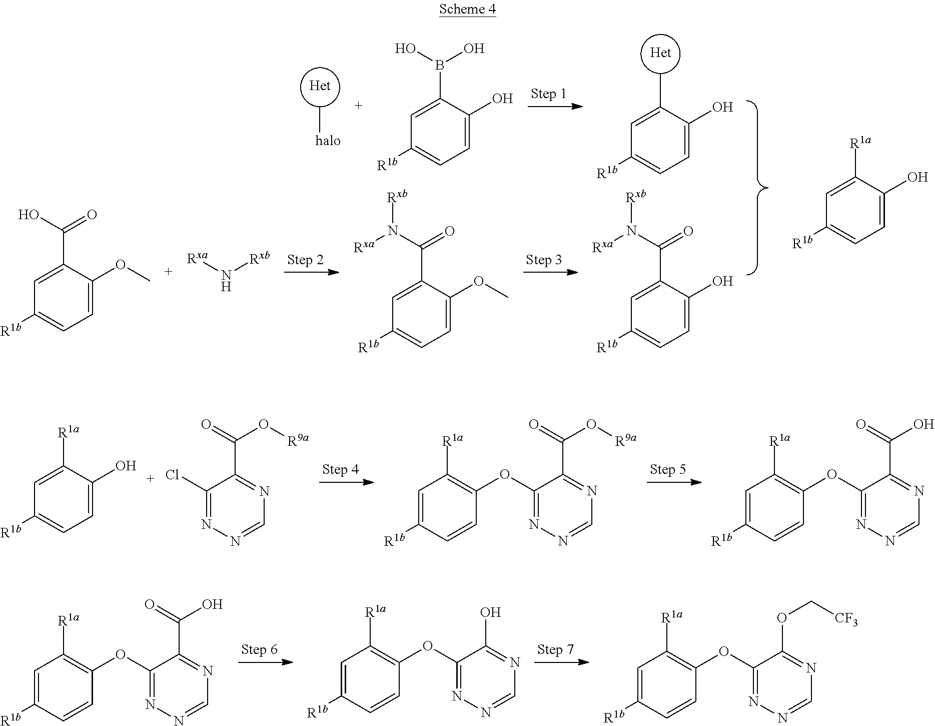

In Scheme 4, the following reaction conditions apply:

Step 1: at a suitable temperature such as for example 90° C., in the presence of a suitable organometallic catalyst such as for example Pd(dppf)Cl₂, in the presence of a suitable base such as for example Na₂CO₃, in suitable solvents such as for example 1,4-dioxane and H₂O;

Step 2: at a suitable temperature such as for example RT, in the presence of suitable amide condensation reagent such as for example HATU, in the presence of a suitable base such as for example DIEA, in a suitable solvent such as for example DCM;

Step 3: at a suitable temperature such as for example from −78° C. to RT, in the presence of a suitable Lewis acid such as for example BBr₃, in a suitable solvent such as for example DCM;

Step 4: at a suitable temperature such as for example from −78° C. to 40° C., in particular from 0° C. to RT, in the presence of a suitable base such as for example TEA, DBU or K₂CO₃, in a suitable solvent such as for example DCM, THF or DMF;

Step 5: at a suitable temperature such as for example RT, in the presence of a suitable base such as for example LiOH·H₂O, in suitable solvents such as for example THF and H₂O;

Step 6: at a suitable temperature such as for example RT, in the presence of a suitable organometallic catalyst such as for example Ag(Phen)₂OTf, in the presence of a suitable brominating reagent such as for example 1,3-dibromo-1,3,5-triazinane-2,4,6-trione, in a suitable solvent such as for example DCE;

Step 7: at a suitable temperature such as for example RT, in the presence of a suitable brominating reagent such as 1,3-dibromo-1,3,5-triazinane-2,4,6-trione, in the presence of 2,2,2-trifluoroethan-1-ol as solvent.

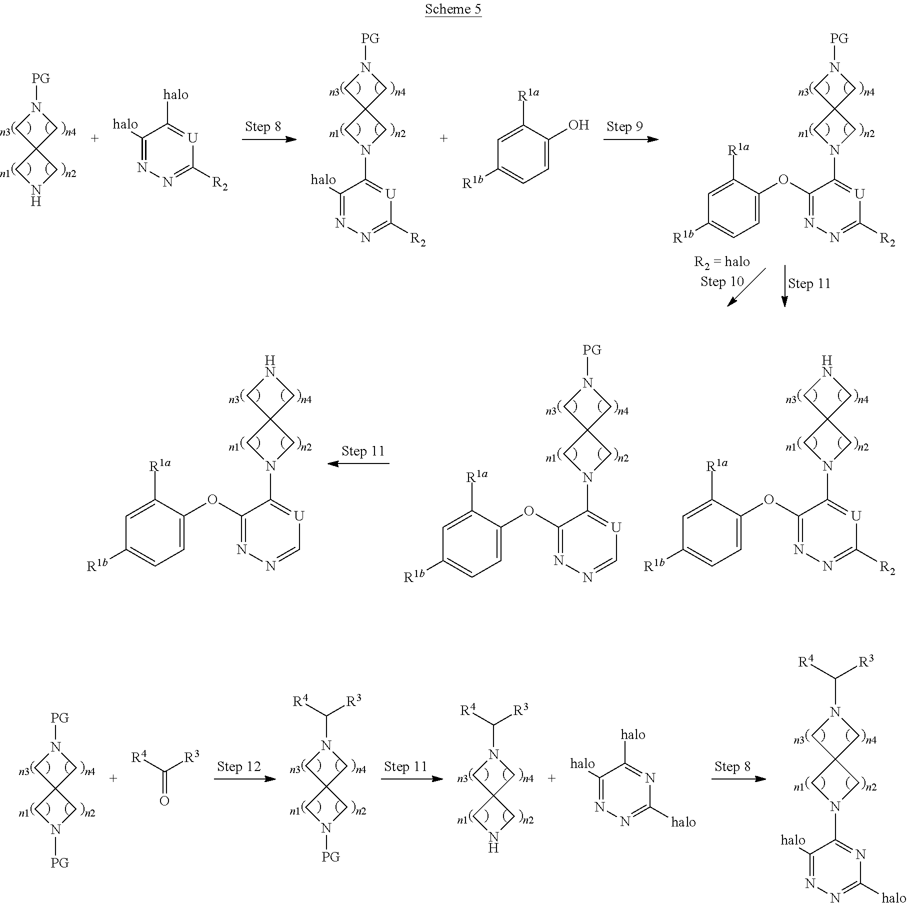

In Scheme 5, the following reaction conditions apply:

Step 8: at a suitable temperature such as for example from −78° C. to 40° C., in particular from 0° C. to RT, in the presence of a suitable base such as for example TEA, DBU or K₂CO₃, in a suitable solvent such as for example DCM, THF or DMF;

Step 9: at a suitable temperature such as for example from −78° C. to 40° C., in particular from 0° C. to RT, in the presence of a suitable base such as for example TEA, DBU or K₂CO₃, in a suitable solvent such as for example DCM, THF or DMF;

Step 10: at a suitable temperature such as for example RT, in the presence of a suitable organometallic catalyst as for example Pd/C and a suitable base as for example TEA, in a suitable solvent such as for example MeOH under H₂ atmosphere;

Step 11: When PG is Boc, at a suitable temperature such as for example RT, in the presence of a suitable acid as for example TFA, in a suitable solvent such as for example DCM.

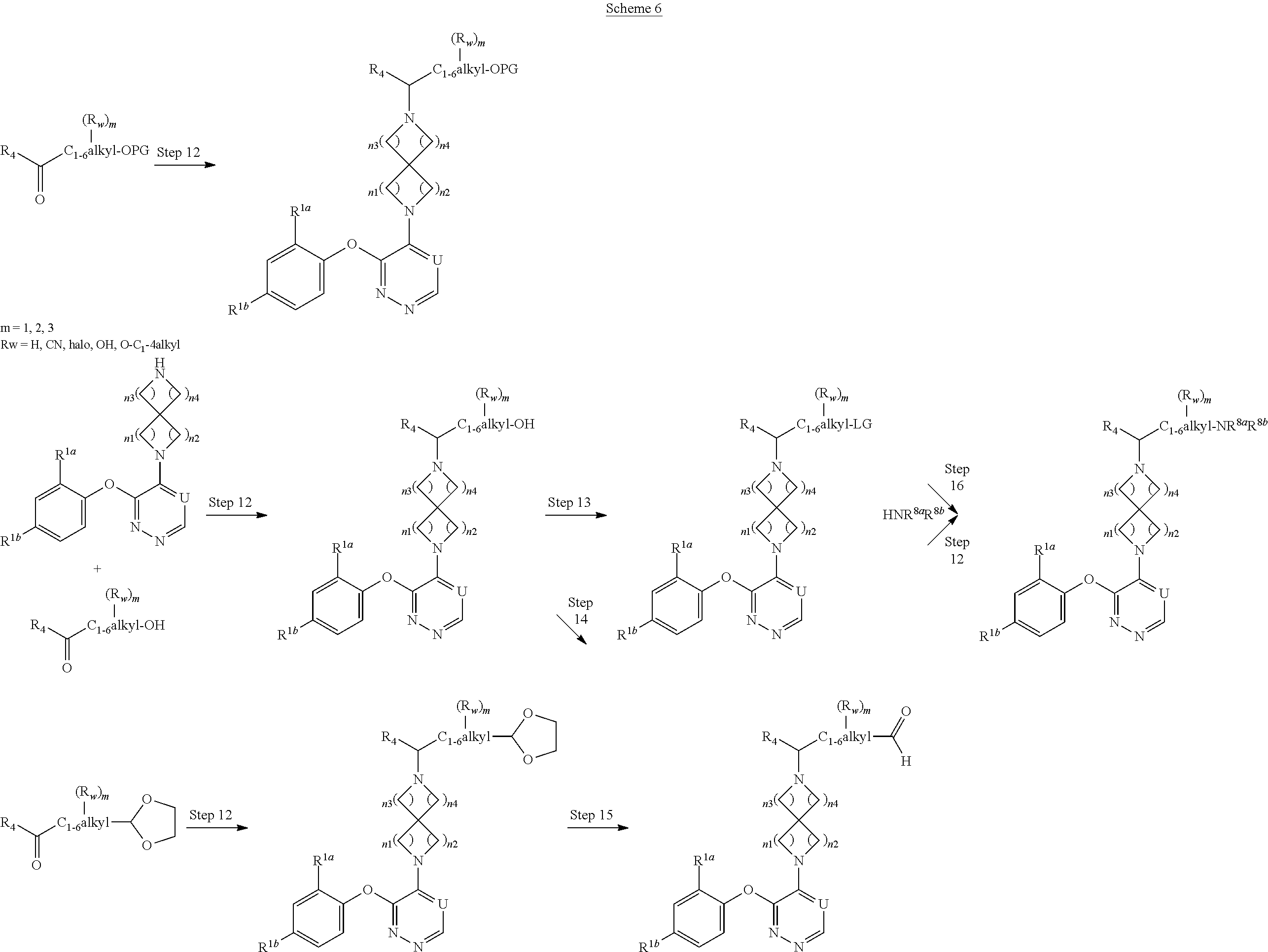

In Scheme 6, the following reaction conditions apply:

Step 12: reductive amination condition, at a suitable temperature such as for example from RT to 80° C., in the presence or absence of a suitable Lewis acid such as for example ZnCl₂ or an acid for example AcOH, in the presence of a suitable reducing agent such as for example NaBH₃CN, in a suitable solvent such as for example MeOH;

Step 13: at a suitable temperature such as for example 0° C., in the presence of a suitable electrophile as for example MsCl, in the presence of a suitable base such as for example TEA, in a suitable solvent such as for example DCM;

Step 14: at a suitable temperature such as for example from 0° C. to RT, in the presence of a suitable oxidizing agent as for example DMP, in a suitable solvent such as for example DCM;

Step 15: at a suitable temperature such as for example 50° C., in the presence of a suitable acid as for example HCl, in a suitable solvent such as for example ACN;

Step 16: at a suitable temperature such as for example RT, in the presence or absence of a suitable base as for example TEA, in a suitable solvent such as for example THF.

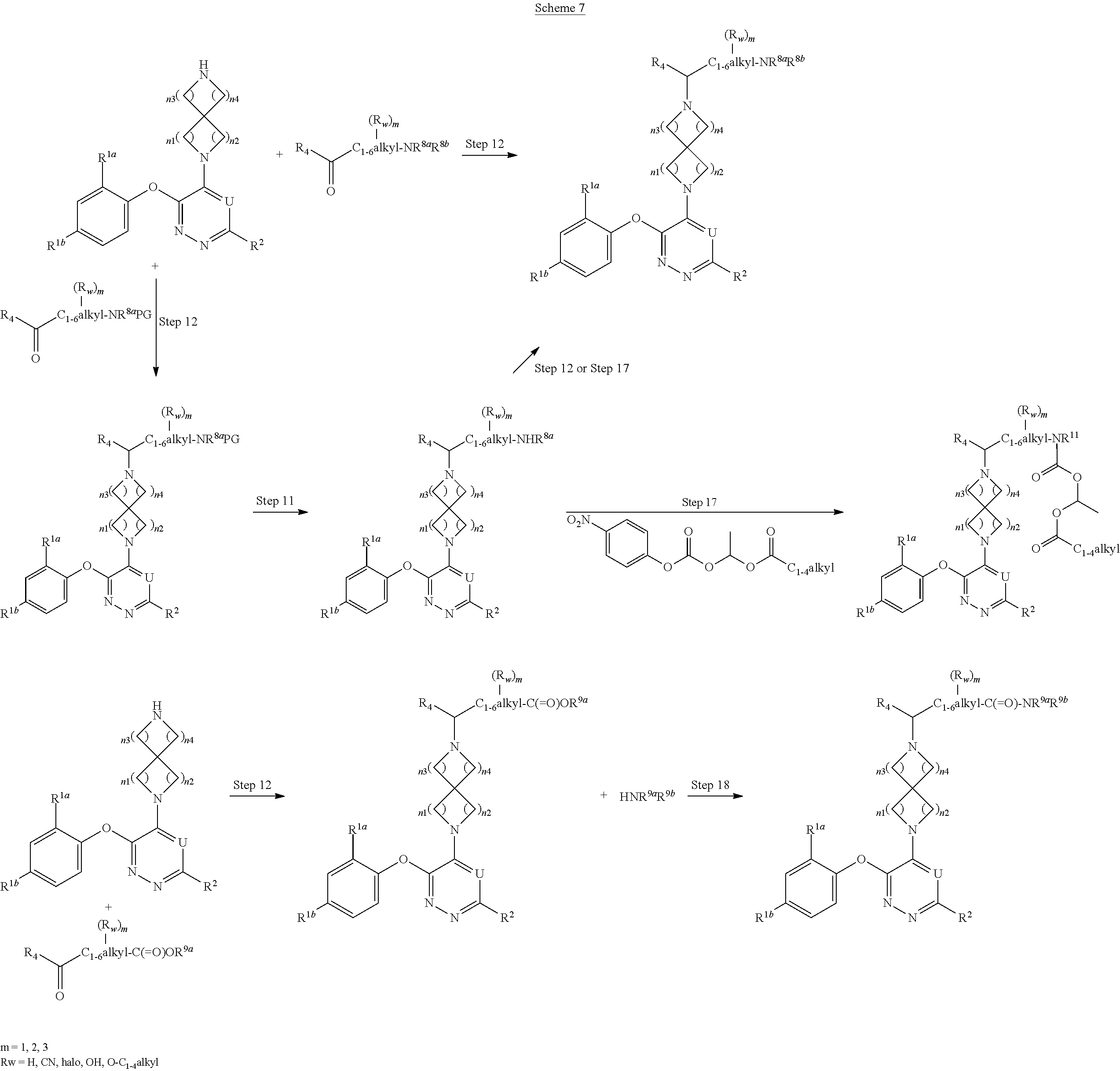

In Scheme 7. the following reaction conditions apply:

Step 11: When PG is Boc, at a suitable temperature such as for example RT, in the presence of a suitable acid as for example TFA, in a suitable solvent such as for example DCM;

Step 12: reductive amination condition, at a suitable temperature such as for example from RT to 80° C., in the presence or absence of a suitable Lewis acid such as for example ZnCl₂ or an acid for example AcOH, in the presence of a suitable reducing agent such as for example NaBH₃CN, in a suitable solvent such as for example MeOH;

Step 17: at a suitable temperature such as for example from RT to 80° C., in the presence of a suitable base such as for example DIEA or Cs₂CO₃, in suitable solvent such as for example DCM or DMF;

Step 18: at a suitable temperature such as for example 40° C., in the presence of a suitable base such as for example ammonia, in suitable solvent such as for 1,4-dioxane.

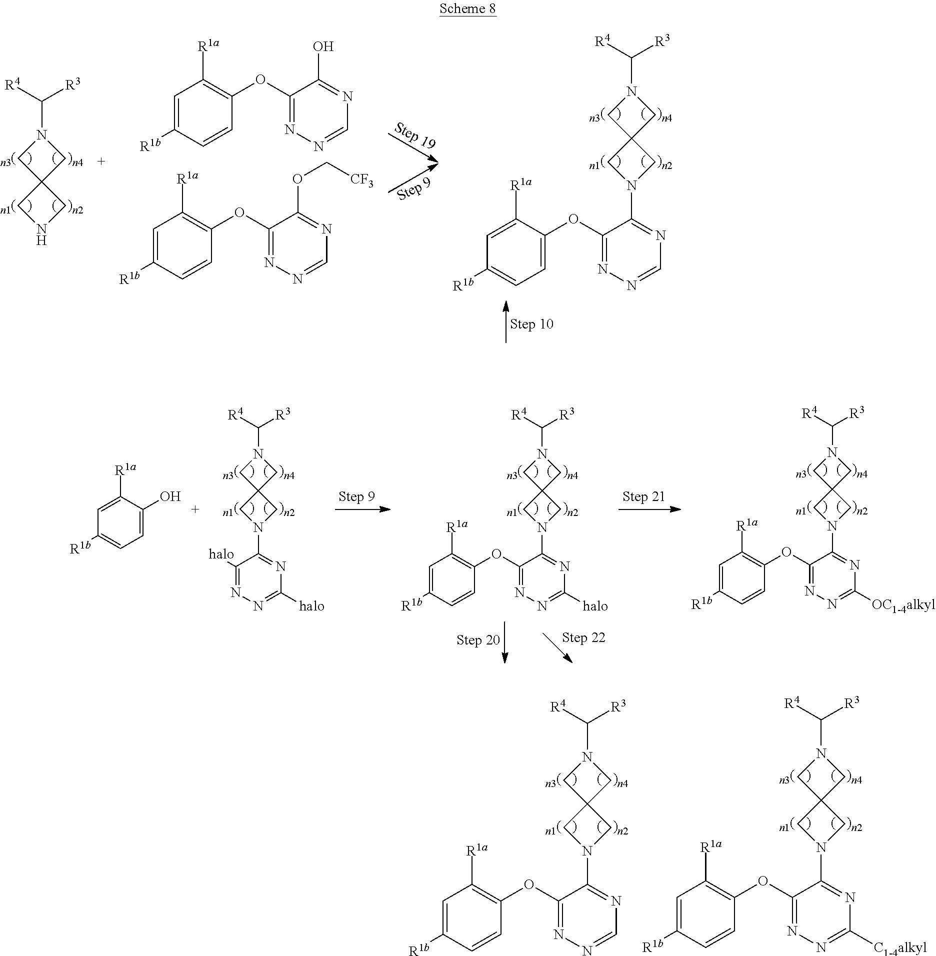

In Scheme 8, the following reaction conditions apply:

Step 9: at a suitable temperature such as for example from −78° C. to 40° C., in particular from 0° C. to RT, in the presence of a suitable base such as for example TEA, DBU or K₂CO₃, in a suitable solvent such as for example DCM. THF or DMF;

Step 10: at a suitable temperature such as for example RT, in the presence of a suitable organometallic catalyst as for example Pd/C, optionally in the presence of a suitable base as for example TEA, in a suitable solvent such as for example MeOH under H₂ atmosphere;

Step 19: at a suitable temperature such as for example RT, in the presence of a suitable chlorinating reagent such as for example oxalyl chloride, in the presence of DMF, in a suitable solvent such as for example DCM;

Step 20: at a suitable temperature such as for example 90° C., in the presence of a suitable nucleophilic amine, in a suitable solvent such as for example EtOH;

Step 21: at a suitable temperature such as for example RT. in the presence of a suitable acid such as for example HCl in dioxane, in a suitable solvent such as for example MeOH;

Step 22: at a suitable temperature such as for example 110° C., in the presence of a suitable boron reagent such as for example trimethylboroxine, in the presence of a suitable organometallic catalyst such as for example tetrakis(triphenylphosphine)palladium(0), in the presence of a suitable base such as for example K₂CO₃, in a suitable solvent such as for example 1,4-dioxane;

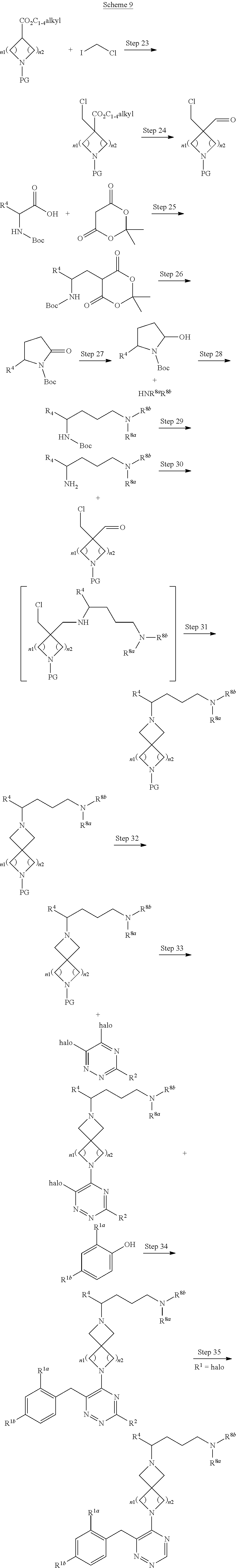

In Scheme 9, the following reaction conditions apply:

Step 23: at a suitable temperature such as for example from −78° C. to −25° C., in the presence of suitable bases such as for example DIEA and n-BuLi, in a suitable solvent such as for example THF;s

Step 24: at a suitable temperature such as for example between −65° C. and −55° C., in the presence of suitable reducing agent such as for example DIBAL-H, in a suitable solvent such as for example toluene, preferably conducted in a suitable flow chemistry system;

Step 25: first at a suitable temperature such as for example from −10° C. to 10° C., in the presence of a suitable base such as for example DMAP, in the presence of a suitable condensation agent such as for example DCC, in a suitable solvent such as for example DCM; then at a suitable temperature such as for example from −10° C. to 0° C., in the presence of a suitable acid such as for example AcOH, in the presence of a suitable reducing agent such as for example NaBH₄, in a suitable solvent such as for example DCM;

Step 26: in a suitable solvent such as for example toluene and heated to reflux;

Step 27: at a suitable temperature such as for example from −5° C. to 5° C., in the presence of 10 suitable reducing agent such as for example LiBH₄, in a suitable solvent such as for example 2-methyltetrahydrofuran;

Step 28: at a suitable temperature such as for example from 15° C. to 25° C., in the presence of a suitable reducing agent such as for example NaBH(OAc)3, in a suitable solvent such as for example DCM;

Step 29: at a suitable temperature such as for example from 15° C. to 25° C., in the presence of a suitable acid such as for HCl, in a suitable solvent such as for example IPA;

Step 30: at a suitable temperature such as for example from 5° C. to 30° C., in the presence of a suitable base such as for example TEA, in the presence of suitable reducing agent such as for example NaBH(OAc)₃, in a suitable solvent such as for example toluene;

Step 31: at a suitable temperature such as for example from 50° C. to 55° C., in the presence of a suitable base such as for example K₂HPO₄, in a suitable solvent such as for example H₂O;

Step 32: When PG is Bn at a suitable temperature such as for example from −5° C. to 45° C., under a hydrogen atmosphere within a suitable pressure range such as for example from 0.27 to 0.40 MPa, in the presence of a suitable catalyst such as for example palladium hydroxide on carbon, in the presence of a suitable acid as for example MSA in a suitable solvent such as EtOH;

Step 33: at a suitable temperature such as for example from −50° C. to −40° C., in the presence of suitable base such as for example TEA, in a suitable solvent such as 2-methyltetrahydrofuran;

Step 34: at a suitable temperature such as for example from 20° C. to 30° C., in the presence of suitable base such as for example TMG, in a suitable solvent such as 2-methyltetrahydrofuran;

Step 35: at a suitable temperature such as for example from 20° C. to 30° C., under a hydrogen atmosphere within a suitable pressure range such as for example from 0.20 to 0.30 Mpa, in the presence of a suitable catalyst such as for example palladium on carbon, in a suitable solvent such as MeOH;

alternatively, at a suitable temperature such as room temperature, in the presence of a suitable catalyst such as for example 1,1′-Bis(diphenylphosphino)ferrocene-palladiurn(II)dichloride dichloromethane complex, a suitable reducing agent such sodium borohydride, a suitable base such as for example N,N,N′N′-tetramethylethylenedianine, in a suitable solvent such as for example tetrahydrofuran.

### Scheme 10

In general, compounds of Formula (I) wherein Y¹ is limited to —CH₂—, and R² is limited to W¹, hereby named compounds of Formula (Ia), can be prepared according to the following reaction Scheme 10. In Scheme 10, W¹ represents chloro, bromo or iodo; all other variables are defined according to the scope of the present invention.

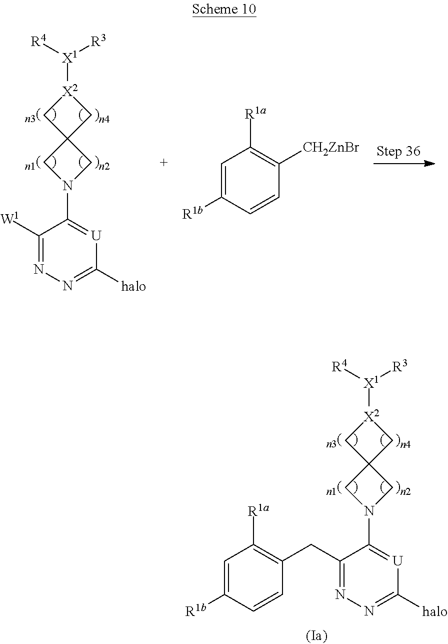

In Scheme 10, the following reaction conditions apply:

Step 36: at a suitable temperature ranged from 60° C. to 100° C., in presence of a suitable catalyst such as palladium acetate (Pd(OAc)₂) or tris(dibenzylideneacetone)dipalladium(0) (Pd₂(dba)₃) or tetrakis(triphenylphosphine)palladium(0), in a suitable solvent such as for example tetrahydrofuran or dioxane.

The skilled person will realize that starting from compound (Ia), analogous chemistry as reported in step 10 in scheme 5 and in steps 20, 21 and 22 in scheme 8 could be performed.

### Scheme 11

In general, compounds of Formula (I) wherein Y¹ is limited to —CR^{5a}R^{5b}— and R² is limited to W¹, hereby named compounds of Formula (Ib), can be prepared according to the following reaction Scheme 11. In Scheme 11 at least one of R^{5a} and R^{5b} is other than hydrogen. All other variables are defined according to the scope of the present invention.

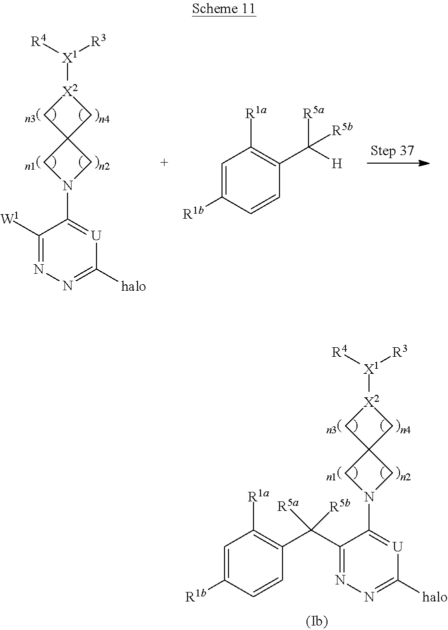

In Scheme 11, the following reaction condition apply:

Step 37: at a suitable temperature ranged from 80° C. to 200° C., in presence of a suitable catalyst such as palladium acetate (Pd(OAc)₂), in the presence of a suitable ligand such as for example triphenylphosphine or tricyclohexylphosphine, in a suitable solvent such as for example dioxane, preferably in sealed conditions, optionally under microwave irradiation.

The skilled person will realize that starting from compound (Ib), analogous chemistry as reported in step 10 in scheme 5 and in steps 20, 21 and 22 in scheme 8 could be performed.

### Scheme 12

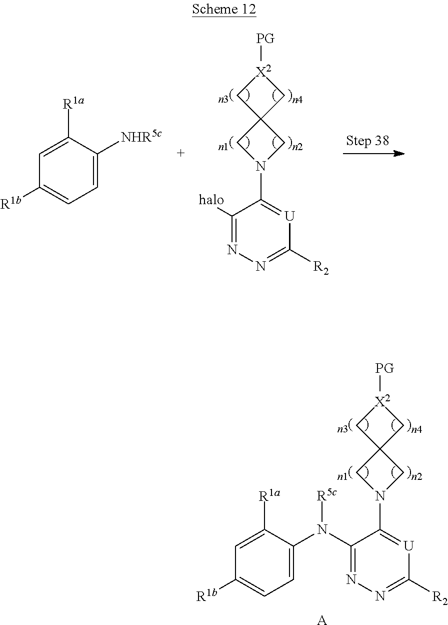

In Scheme 12, the following reaction condition apply:

Step 38: at a suitable temperature such as for example from RT to 80° C., in the presence of a suitable base such as for example DIEA, Cs₂CO₃ or DBU, in suitable solvent such as for example DCM, THF or DMF;

Alternatively, at a suitable temperature such as for example RT to 100° C., in the presence of a suitable catalyst such as for example Pd₂dba₃, in the presence of a suitable ligand such as for example Xantphos, in the presence of a suitable base such as Cs₂CO₃ or Na₂CO₃, in a suitable solvent such dioxane or a mixture of dioxane and water

The skilled person will realize that starting from intermediate A, analogous chemistry as reported in case Y¹ represents O can be performed.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation. sulfonylation. halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl(CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, New Jersey, 2007.

### Pharmacology

It has been found that the compounds of the present invention block the interaction of menin with MLL proteins and oncogenic MLL fusion proteins per se, or can undergo metabolism to a (more) active form in vivo (prodrugs). Therefore the compounds according to the present invention and the pharmaceutical compositions comprising such compounds may be useful for the treatment or prevention, in particular treatment, of diseases such as cancer, including but not limited to leukemia, myelodysplastic syndrome (MDS), and myeloproliferative neoplasms (MPN); and diabetes.

In particular, the compounds according to the present invention and the pharmaceutical compositions thereof may be useful in the treatment or prevention of cancer. According to one embodiment, cancers that may benefit from a treatment with menin/MLL inhibitors of the invention comprise leukemias, lymphomas, myelomas or solid tumor cancers (e.g. prostate cancer, lung cancer, breast cancer, pancreatic cancer, colon cancer, liver cancer, melanoma and glioblastoma, etc.). In some embodiments, the leukemias include acute leukemias, chronic leukemias, myeloid leukemias, myelogeneous leukemias, lymphoblastic leukemias, lymphocytic leukemias, Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, leukemias exhibiting HOX1ME1S1 gene expression signatures etc.

In particular, the compounds according to the present invention and the pharmaceutical compositions thereof may be useful in the treatment or prevention of myelodysplastic syndrome (MDS) or myeloproliferative neoplasms (MPN).

In particular, compounds according to the present invention and the pharmaceutical compositions thereof may be useful in the treatment or prevention of leukemias, in particular nucleophosmin (NPMI)-nutated leukemias, e.g. NPM1c.

In particular, compounds according to the present invention and the pharmaceutical compositions thereof may be useful in the treatment or prevention of AML, in particular nucleophosmin (NPMI)-mutated AML (i.e., NPM1^{mut} AML), more in particular abstract NPM1-mutated AML.

In particular, compounds according to the present invention and the pharmaceutical compositions thereof may be useful in the treatment or prevention of MLL-rearranged leukemias, in particular MLL-rearranged AML or ALL.

In particular, compounds according to the present invention and the pharmaceutical compositions thereof may be useful in the treatment or prevention of leukemias with MLL gene alterations, in particular AML or ALL with MLL gene alterations.

In particular, compounds according to the present invention and the pharmaceutical compositions thereof may be suitable for Q.D. dosing (once daily).

In particular, compounds according to the present invention and the pharmaceutical compositions thereof may be useful in the treatment or prevention of hematological cancer in a subject exhibiting NPM1 gene mutations and/or mixed lineage leukemia gene (MILL; MLL1; KMT2A) alterations, mixed lineage leukemia (MLL), MLL-related leukemia, MLL-associated leukemia, MLL-positive leukemia, MLL-induced leukemia, rearranged mixed lineage leukemia, leukemia associated with a MLL, rearrangement/alteration or a rearrangement/alteration of the MLL gene, acute leukemia, chronic leukemia, myelodysplastic syndrome (MDS), myeloproliferative neoplasms (MPN), insulin resistance, pre-diabetes, diabetes, or risk of diabetes, hyperglycemia, chromosomal rearrangement on chromosome 11q23, type-1 diabetes, type-2 diabetes; promoting proliferation of a pancreatic cell, where pancreatic cell is an islet cell, beta cell, the beta cell proliferation is evidenced by an increase in beta cell production or insulin production; and for inhibiting a menin-MLL interaction, where the MLL fusion protein target gene is HOX or MEIS1 in human.

Hence, the invention relates to compounds of Formula (I), the tautomers and the stereoisomeric forms thereof, and the pharmaceutically acceptable salts, and the solvates thereof, for use as a medicament.

The invention also relates to the use of a compound of Formula (I), a tautomer or a stereoisomeric form thereof, or a pharmaceutically acceptable salt, or a solvate thereof, or a pharmaceutical composition according to the invention, for the manufacture of a medicament.

The present invention also relates to a compound of Formula (I), a tautomer or a stereoisomeric form thereof, or a pharmaceutically acceptable salt, or a solvate thereof, or a pharmaceutical composition according to the invention, for use in the treatment, prevention, amelioration, control or reduction of the risk of disorders associated with the interaction of menin with MLL proteins and oncogenic MLL fusion proteins in a mammal, including a human, the treatment or prevention of which is affected or facilitated by blocking the interaction of menin with MLL proteins and oncogenic MLL fusion proteins.

Also, the present invention relates to the use of a compound of Formula (I), a tautomer or a stereoisomeric form thereof, or a pharmaceutically acceptable salt, or a solvate thereof, or a pharmaceutical composition according to the invention, for the manufacture of a medicament for treating, preventing, ameliorating, controlling or reducing the risk of disorders associated with the interaction of menin with MLL proteins and oncogenic MLL fusion proteins in a mammal, including a human, the treatment or prevention of which is affected or facilitated by blocking the interaction of menin with MLL proteins and oncogenic MLL fusion proteins.

The invention also relates to a compound of Formula (I), a tautomer or a stereoisomeric form thereof, or a pharmaceutically acceptable salt, or a solvate thereof, for use in the treatment or prevention of any one of the diseases mentioned hereinbefore.

The invention also relates to a compound of Formula (I), a tautomer or a stereoisomeric form thereof, or a pharmaceutically acceptable salt, or a solvate thereof, for use in treating or preventing any one of the diseases mentioned hereinbefore.

The invention also relates to the use of a compound of Formula (I), a tautomer or a stereoisomeric form thereof, or a pharmaceutically acceptable salt, or a solvate thereof, for the manufacture of a medicament for the treatment or prevention of any one of the disease conditions mentioned hereinbefore.

The compounds of the present invention can be administered to mammals, preferably humans, for the treatment or prevention of any one of the diseases mentioned hereinbefore.

In view of the utility of the compounds of Formula (I), the tautomers and the stereoisomeric forms thereof, and the pharmaceutically acceptable salts, and the solvates thereof, there is provided a method of treating warm-blooded animals, including humans, suffering from any one of the diseases mentioned hereinbefore.

Said method comprises the administration, i.e. the systemic or topical administration, of a therapeutically effective amount of a compound of Formula (I), a tautomer or a stereoisomeric form thereof, or a pharmaceutically acceptable salt, or a solvate thereof, to warm-blooded animals, including humans.

Therefore, the invention also relates to a method for the treatment or prevention of any one of the diseases mentioned hereinbefore comprising administering a therapeutically effective amount of compound according to the invention to a patient in need thereof. One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. An effective therapeutic daily amount would be from about 0.005 mg/kg to 100 mg/kg. The amount of a compound according to the present invention, also referred to herein as the active ingredient, which is required to achieve a therapeutically effect may vary on case-by-case basis, for example with the particular compound, the route of administration, the age and condition of the recipient, and the particular disorder or disease being treated. A method of treatment may also include administering the active ingredient on a regimen of between one and four intakes per day. In these methods of treatment the compounds according to the invention are preferably formulated prior to administration.

The present invention also provides compositions for preventing or treating the disorders referred to herein. Said compositions comprising a therapeutically effective amount of a compound of Formula (I), a tautomer or a stereoisomeric form thereof, or a pharmaceutically acceptable salt, or a solvate thereof, and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions may be prepared by any methods well known in the art of pharmacy, for example, using methods such as those described in Gennaro et al. Remington's Pharmaceutical Sciences (18^{th} ed., Mack Publishing Company, 1990, see especially Part 8 Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, an embodiment of the present invention relates to a product containing as first active ingredient a compound according to the invention and as further active ingredient one or more anticancer agent, as a combined preparation for simultaneous, separate or sequential use in the treatment of patients suffering from cancer.

The one or more other medicinal agents and the compound according to the present invention may be administered simultaneously (e.g. in separate or unitary compositions) or sequentially in either order. In the latter case, the two or more compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular other medicinal agent and compound of the present invention being administered, their route of administration, the particular condition, in particular tumour, being treated and the particular host being treated.

The following examples further illustrate the present invention.

### Examples

Several methods for preparing the compounds of this invention are illustrated in the following examples. Unless otherwise noted, all starting materials were obtained from commercial suppliers and used without further purification, or alternatively can be synthesized by a skilled person bv using well-known methods

| Abbreviation | Meaning |
|---|---|
| Ag(Phen)₂OTf | silver triflate-bis(1,10-phenanthroline) complex |
| 2-MeTHF | 2-methyltetrahydrofuran |
| ACN | acetonitrile |
| AcOH or HOAc | acetic acid |
| aq. | aqueous |
| Ar | argon |
| BBr₃ | tribromoborane |
| bn | benzyl |
| Boc | tert-butyloxycarbonyl |
| Boc₂O | di-tert-butyl dicarbonate |
| n-BuLi | n-butyllithium |
| Cbz | benzyloxycarbonyl |
| CHCl₃ | chloroform |
| Cs₂CO₃ | cesium carbonate |
| conc. | concentrated |
| DBU | 1,8-diazabicyclo[5.4.0]undec-7-ene |
| DCC | dicyclohexylcarbodiimide |
| DCE | dichloroethane |
| DCM | dichloromethane |
| DEA | diethylamine |
| DIBAL-H | diisobutylaluminum hydride |
| DIEA or DIPEA | N,N-diisopropylethylamine |
| DMAP | N,N-dimethylpyridin-4-amine |
| DMF | N,N-dimethylformamide |
| DMP | Dess-Martin periodinane |
| DMSO | dimethyl sulfoxide |
| dppf | 1,1′-ferrocenediyl-bis(diphenylphosphine) |
| EDCI | N-(3-Dimethylaminopropyl)-N′-ethylcarbodiimide |
| | hydrochloride |
| EA or EtOAc | ethyl acetate |
| EtOH | ethanol |
| eq. | equivalent(s) |
| FA | formic acid |
| FCC | flash column chromatography |
| h | hour(s) |
| H₂ | hydrogen |
| HATU | 1-[bis(dimethylamino)methylene]-1H-1,2,3- |
| | triazolo[4,5-b]pyridinium 3-oxid |
| | hexafluorophosphate |
| H₂O | water |
| HCl | hydrochloric acid |
| HOBt | 1-Hydroxybenzotriazole |
| HPLC | high performance liquid chromatography |
| IPA | isopropyl alcohol |
| IPAc | isopropyl acetate |
| K₂CO₃ | potassium carbonate |
| KI | potassium iodide |
| K₂HPO₄ | dipotassium phosphate |
| K₃PO₄ | tripotassium phosphate |
| LCMS | liquid chromatography mass spectrometry |
| LiAID₄ | lithium aluminum deuteride |
| LAH | lithium aluminum hydride |
| LiBH₄ | lithium borohydride |
| LDA | lithium diisopropylamide |
| LiCl | lithium chloride |
| LG | leaving group |
| Me | methyl |
| MeOH | methanol |
| 2-MeTHF | 2-methyltetrahydrofuran |
| min or mins | minute(s) |
| mL | milliliters |
| mmol | millimoles |
| mg | milligram |
| MgSO₄ | magnesium sulfate |
| MSA | methanesulfonic acid |
| MsCl | methanesulfonyl chloride |
| MTBE | methyl tert-butyl ether |
| N₂ | nitrogen |
| NA | not available |
| NaBH₃CN | sodium cyanoborohydride |
| NaBH(OAc)₃ | sodium triacetoxyborohydride |
| NaBD₃CN | sodium cyanoborodeuteride |
| Na₂CO₃ | sodium carbonate |
| NaH | sodium hydride |
| NaHCO₃ | sodium bicarbonate |
| NaI | sodium iodide |
| NaOAc orAcONa | sodium acetate |
| NaOH | sodium hydroxide |
| Na₂SO₃ | sodium sulfite |
| Na₂SO₄ | sodium sulfate |
| NBS | N-bromosuccinimide |
| NH₄Cl | ammonium chloride |
| NMM | 1-4-Methylmorpholine |
| Pd₂dba₃ | tris(dibenzylideneacetone)dipalladium(0) |
| Pd(dppf)Cl₂•DCM | [1,1′- |
| | bis(diphenylphosphino)ferrocene]dichloro- |
| | palladium(II), complex with dichloromethane |
| Pd(PPh₃)₄ | tetrakis(triphenylphosphine)palladium(0) |
| PE | petroleum ether |
| PG | protecting group |
| Phen | phenanthroline |
| psi | pound per square inch |
| p-TsOH | p-toluenesulfonic acid |
| p-TsOH•H₂O | p-toluenesulfonic acid monohydrate |
| PyBrop | bromotripyrrolidinophosphonium |
| | hexafluorophosphate |
| Rf | retention factor |
| Rₜ | retention time |
| RT or rt or r.t. | room temperature |
| sat. | saturated |
| SFC | supercritical fluid chromatography |
| TBAF | tetrabutylammonium fluoride |
| TBDMS | tert-butyldimethylsilyl |
| TBDPS | tert-butyldiphenylsilyl |
| t-BuOK | potassium tert-butoxide |
| TFA | trifluoroacetic acid |
| T₃P | propanephosphonic acid anhydride |
| TEA or Et₃N | triethylamine |
| Tf | trifluoromethanesulfonyl |
| TFA | trifluoroacetic acid |
| THF | tetrahydrofuran |
| Ti(OiPr)₄ | titanium(IV) isopropoxide |
| TLC | thin layer chromatography |
| TMEDA | N,N,N′,N′-tetramethylethylenediamine |
| TMG | 1,1,3,3-tetramethylguanidine |
| TMSI | iodotrimethylsilane |
| Ts | p-toluenesulfonyl |
| TsCl | p-toluenesulfonyl chloride |
| v/v | volume per volume |
| vol. | volume(s) |
| wt | weight |
| Xantphos | 4,5-bis(diphenylphosphino)-9,9-dimethylxanthene |

As understood by a person skilled in the art, compounds synthesized using the protocols as indicated may exist as a solvate e.g. hydrate, and/or contain residual solvent or minor impurities. Compounds or intermediates isolated as a salt form, may be integer stoichiometric i.e. mono- or di-salts, or of intermediate stoichiometry. When an intermediate or compound in the experimental part below is indicated as ‘HCl salt’ without indication of the number of equivalents of HCl, this means that the number of equivalents of HCl was not determined. The same principle will also apply to all other salt forms referred to in the experimental part, such as e.g.

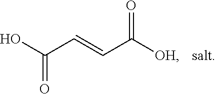

The stereochemical configuration for centers in some compounds may be designated “R” or “S” when the mixture(s) was separated; for some compounds, the stereochemical configuration at indicated centers has been designated as “*R” (first eluted from the column in case the column conditions of the separation are described in the synthesis protocol and when only one stereocenter present or indicated) or “*S” (second eluted from the column in case the column conditions of the separation are described in the synthesis protocol and when only one stereocenter present or indicated) when the absolute stereochemistry is undetermined (even if the bonds are drawn stereo specifically) although the compound itself has been isolated as a single stereoisomer and is enantiomerically pure.

For example, it will be clear that Compound 11

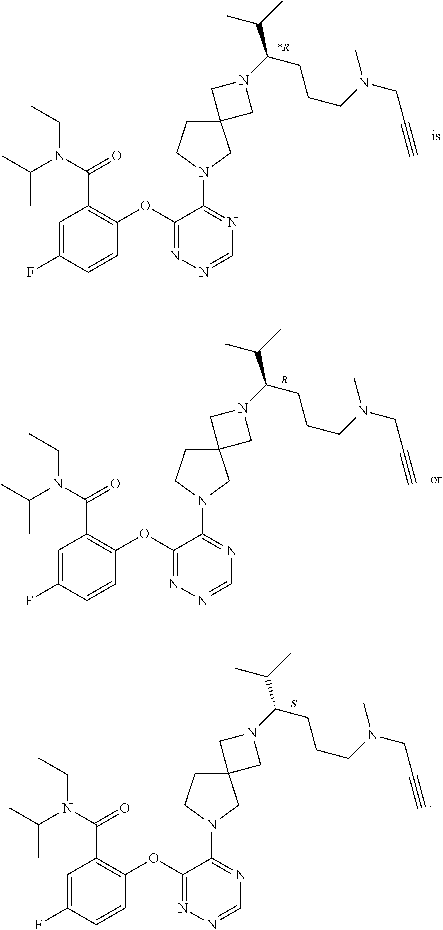

For compounds wherein the stereochemical configuration of two stereocentres is indicated by * (e.g. *R or *S), the absolute stereochemistry of the stereocentres is undetermined (even if the bonds are drawn stereospecifically), although the compound itself has been isolated as a single stereoisomer and is enantiomerically pure. In this case, the configuration of the first stereocentre is independent of the configuration of the second stereocentre in the same compound. “*R” or “*S” is assigned randomly for such molecules.

For example, for Compound 24

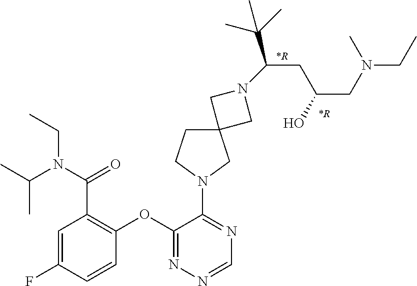 [SMILES: CCN(C)C[C@H](O)C[C@@H](N1CC2(CCN(c3ncnnc3Oc3ccc(F)cc3C(=O)N(CC)C(C)C)C2)C1)C(C)(C)C]

this means that the compound is

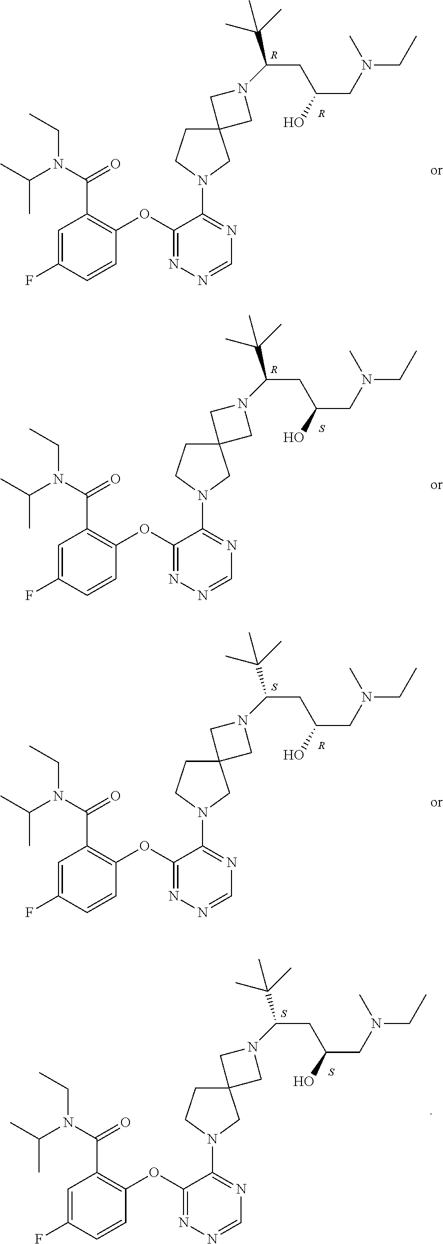

A skilled person will realize that the paragraphs above about stereochemical configurations, also apply to intermediates.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

When a stereocenter is indicated with ‘RS’ this means that a racemic mixture was obtained at the indicated centre, unless otherwise indicated.

Preparation of intermediates

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

### Preparation of intermediate 1

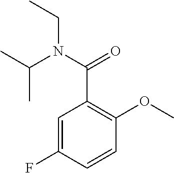 [SMILES: CCN(C(=O)c1cc(F)ccc1OC)C(C)C]

To the mixture of 5-fluoro-2-methoxybenzoic acid (8.00 g, 47.0 mmol) and N-ethylpropan-2-amine (8.19 g, 94.0 mmol) in dry DCM (150 mL) cooled at 0° C., were slowly added HATU (21.5 g, 56.5 mmol) and DIEA (9.10 g, 70.4 mmol) in portions. The resulting mixture was slowly warmed to RT and stirred for 8 h. The organic layer was washed with water (20 mL×3) and dried over anhydrous Na₂SO₄. After filtration, the solvent was removed under reduced pressure and the crude product was purified by FCC (EtOAc/PE=0% to 20%) to afford the title intermediate 1 (12.0 g, 96% yield) as a white solid.

The following intermediate was synthesized by an analogous method as described above for intermediate 1

| Int. No. | Structure | Starting Materials |
|---|---|---|
| 2 | 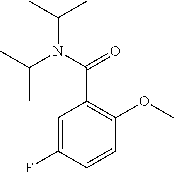 [SMILES: COc1ccc(F)cc1C(=O)N(C(C)C)C(C)C] | 5-fluoro-2-methoxybenzoic acid, diisopropylamine |

### Preparation of intermediate 3

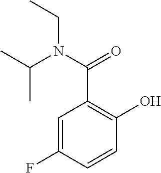 [SMILES: CCN(C(=O)c1cc(F)ccc1O)C(C)C]

To the solution of intermediate 1 (12.0 g, 50.1 mmol) in dry DCM (100 mL) cooled at −78° C. was slowly added BBr₃ (14.4 mL, 152 mmol), the resulting mixture was slowly warmed to RT and stirred for 8 h. The mixture was cooled to −78° C. again and MeOH (5 mL) was added dropwise to quench the reaction. The resulting mixture was slowly warmed to RT and the pH value was adjusted to about 8 by adding sat. aq. NaHCO3 solution. The aqueous layer was extracted by DCM (50 mL×3) and the combined organic layers were dried over anhydrous Na₂SO₄, filtered and concentrated under reduced pressure to give the crude product which was purified by FCC (EtOAc/PE=0% to 20%) to afford the title intermediate 2 (9.0 g, 78% yield) as a white solid.

The following intermediate was synthesized by an analogous method as described above for intermediate 3

| Int. No. | Structure | Starting Materials |
|---|---|---|
| 4 | 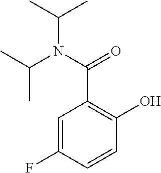 [SMILES: CC(C)N(C(=O)c1cc(F)ccc1O)C(C)C] | intermediate 2 |

### Preparation of intermediate 5

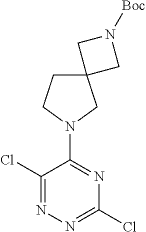 [SMILES: CC(C)(C)OC(=O)N1CC2(CCN(c3nc(Cl)nnc3Cl)C2)C1]

To the solution of 3,5,6-trichloro-1,2,4-triazine (10.0 g, 54.2 mmol) and TEA (15.2 mL, 109 mmol) in DCM (100 mL) cooled at 0° C. was added tert-butyl 2,6-diazaspiro[3.4]octane-2-carboxylate (9.21 g, 43.4 mmol), the mixture was warmed to RT and stirred for 1 h. The mixture was diluted with water (20 mL) and extracted with DCM (30 mL×3). The combined organic layers were washed with brine, dried over Na₂SO₄, filtered and concentrated under reduced pressure to give the crude product which was purified by FCC on silica gel (PE/EtOAc=1:0 to 3:1) to afford the title intermediate 3 (12.0 g, 58% yield) as a yellow solid.

### Preparation of intermediate 6

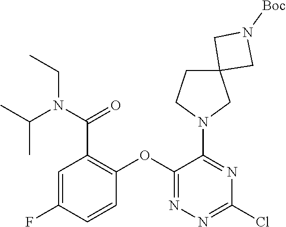 [SMILES: CCN(C(=O)c1cc(F)ccc1Oc1nnc(Cl)nc1N1CCC2(CN(C(=O)OC(C)(C)C)C2)C1)C(C)C]

The mixture of intermediate 5 (12.0 g, 33.3 mmol), intermediate 2 (7.5 g, 33.3 mmol) and DBU (6.1 g, 40.1 mmol) in THF (120 mL) was stirred at 25° C. for 8 h. The mixture was diluted with water (30 mL) and extracted with DCM (30 mL×3). The combined organic layers were washed with brine, dried over Na₂SO₄, filtered and concentrated under reduced pressure to give the crude product which was purified by FCC (PE/EtOAc=1:0 to 3:1) to afford the title intermediate 4 (14.0 g, 73% yield) as green solid.

The following intermediate was synthesized by an analogous method as described above for intermediate 6

| Int. No. | Structure | Starting Materials |
|---|---|---|
| 7 | 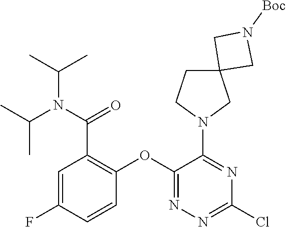 [SMILES: CC(C)N(C(=O)c1cc(F)ccc1Oc1nnc(Cl)nc1N1CCC2(CN(C(=O)OC(C)(C)C)C2)C1)C(C)C] | intermediate 5 & intermediate 4 |

### Preparation of intermediate 8

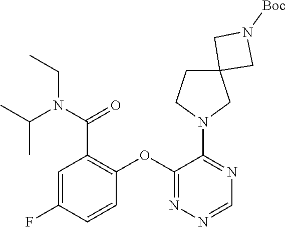 [SMILES: CCN(C(=O)c1cc(F)ccc1Oc1nncnc1N1CCC2(CN(C(=O)OC(C)(C)C)C2)C1)C(C)C]

To the mixture of intermediate 6 (20 g, 36.4 mmol), NaBH₄ (2.48 g, 65.7 mmol) and TMEDA (8.54 g, 73.5 mmol) in THF (500 mL) was added Pd(dppf)Cl₂·DCM (1.70 g, 2.08 mmol) under N₂ atmosphere. After addition, the reaction mixture was stirred at 25° C. for 14 h. The reaction mixture was filtered and the filtrate was concentrated, the residue was purified by FCC on silica gel (EtOAc) to afford the title intermediate 5 (15 g, 93% purity, 74% yield) as brown solid.

The following intermediates were synthesized by an analogous method as described above for intermediate 8

| Int. No. | Structure | Starting Materials |
|---|---|---|
| 9 | 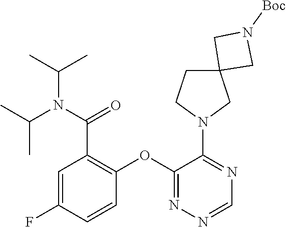 [SMILES: CC(C)N(C(=O)c1cc(F)ccc1Oc1nncnc1N1CCC2(CN(C(=O)OC(C)(C)C)C2)C1)C(C)C] | intermediate 7 |
| 36 | 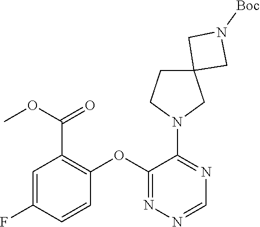 [SMILES: COC(=O)c1cc(F)ccc1Oc1nncnc1N1CCC2(CN(C(=O)OC(C)(C)C)C2)C1] | methyl 5-fluoro-2-hydroxybenzoate |

### Preparation of intermediate 10

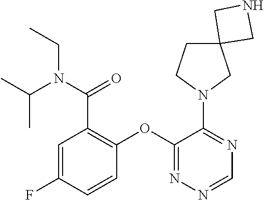 [SMILES: CCN(C(=O)c1cc(F)ccc1Oc1nncnc1N1CCC2(CNC2)C1)C(C)C]

To the solution of intermediate 8 (300 mg, 0.583 mmol) in DCM (5 mL) was added TFA (0.5 mL, 6.4 mmol), the resulting mixture was stirred at RT for 3 h. Then 10% NaOH (5 mL) solution was slowly added into the mixture to adjust the pH value to about 12. the resulting mixture was extracted with DCM (10 mL×3). The combined organic layers were dried over anhydrous Na₂SO₄, filtered, and concentrated in vacuo to afford the title intermediate 6 (220 mg, 90% yield) as a white solid.

The following intermediate was synthesized by an analogous method as described above for intermediate 10

| Int. No. | Structure | Starting Materials |
|---|---|---|
| 11 | 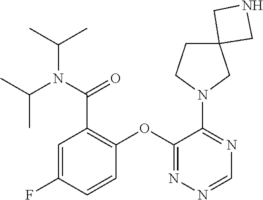 [SMILES: CC(C)N(C(=O)c1cc(F)ccc1Oc1nncnc1N1CCC2(CNC2)C1)C(C)C] | intermediate 9 |

### Preparation of intermediate 12

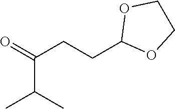

To a solution of Mg (7.15 g, 294 mmol) and 12 (0.105 g, 0.414 mmol) in THF (176 ml) at 50° C. under N₂. The reaction mixture was added dropwise to a solution (10 ml) of 2-(2-bromoethyl)-1,3-dioxolane (25.0 g, 138 mmol, in THF (100 ml)). The color of the mixed system changes from brown to colorless, and the color recovers to room temperature. Then the reaction mixture was added dropwise to a solution (90 ml) of 2-(2-bromoethyl)-1,3-dioxolane (25.0 g, 138 mmol, in THF (100 ml)) at 25° C. for 1 hours. Finally get the crude product was added dropwise to a solution of N-methoxy-N-methylisobutyramide (11.9 g, 90.7 mmol) in THF (100 ml) at 25° C. for 16 under N₂. TLC (PE/EA=4/1, Rf=0.3) showed a new spot was found. Then 300 mL of sat. NH₄Cl (aq.) was added to the mixture to quench the reaction. The mixture was warmed to room temperature and filtered. The filtrate was extracted with EA (300 mL×3), dried over Na₂SO₄ and filtered. The filtrated was evaporated to dryness which was purified with FCC (PE/EA=4/1) to give intermediate 13 (22.0 g, 80% purity) as colorless oil.

The following intermediate was synthesized by an analogous method as described above for intermediate 12

| Int. No. | Structure | Starting Materials |
|---|---|---|
| 13 | 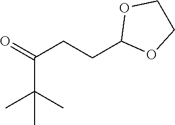 | N-methoxy-N-methylpivalamide |

### Preparation of intermediate 14, 14a & 14b

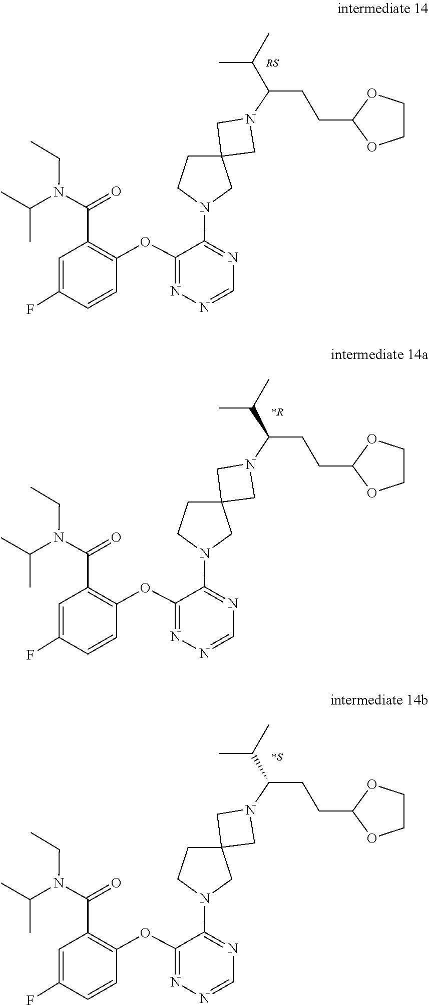 [SMILES: CCN(C(=O)c1cc(F)ccc1Oc1nncnc1N1CCC2(C1)CN(C(CCC1OCCO1)C(C)C)C2)C(C)C ; CCN(C(=O)c1cc(F)ccc1Oc1nncnc1N1CCC2(C1)CN([C@H](CCC1OCCO1)C(C)C)C2)C(C)C ; CCN(C(=O)c1cc(F)ccc1Oc1nncnc1N1CCC2(C1)CN([C@@H](CCC1OCCO1)C(C)C)C2)C(C)C]

A stir bar, intermediate 10 (1.5 g, 3.62 mmol), intermediate 12 (2.7 g, 15.7 mmol), acetic acid (500 mg, 8.33 mmol) and methanol (20 mL) were added to a 100 mL round-bottomed flask, the mixture was heated and stirred at 45° C. for 1 hour before sodium cyanoborohydride (500 mg, 7.96 mmol) was added to the mixture, the mixture was heated and stirred at 45° C. for 8 hours.

The reaction mixture was cooled to the room temperature, and was diluted with dichloromethane (20 mL). Subsequently, a saturated solution of sodium bicarbonate (40 mL) was added, and the mixture was extracted with dichloromethane (20 mL×3). The combined organic layers were dried over anhydrous Na₂SO₄, filtered and concentrated under reduced pressure to give a residue, which was purified by FCC (eluent: dichloromethane: methanol=1:0 to 10:1, dichloromethane: methanol=10:1, Rf=0.4) to give intermediate 14 as colorless oil (1.5 g, 96.81% purity, 70.31% yield).

Intermediate 14 was separated by SFC (separation condition: DAICEL CHIRALPAK IG (250 mm*50 mm ,10 um); Mobile phase: A: Supercritical CO₂, B: 0.1% NH₃H₂O MEOH, A:B =55:45 at 200 mL/min; Column Temp: 38; Nozzle Pressure: 100 Bar; Nozzle Temp: 60; Evaporator Temp: 20; Trimmer Temp: 25; Wavelength: 220 nm). The pure fractions were collected, and the solvent was evaporated under vacuum to give two residues. The first residue one was partitioned between acetonitrile (2 mL) and water (10 mL). The solution was lyophilized to dryness to give intermediate 14a (2.0 g, 99.33% purity, 39.73% yield) as a white solid. The second residue was partitioned between acetonitrile (2 mL) and water (10 mL). The mixture was lyophilized to dryness to give intermediate 14b (2.0 g, 99.79% purity, 39.92% yield) as a white solid.

The following intermediate was synthesized by an analogous method as described above for intermediate 14

| Int. No. | Structure | Starting Materials |
|---|---|---|
| 15 | 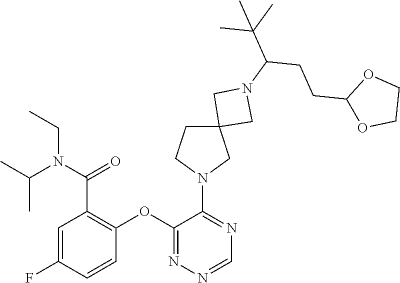 [SMILES: CCN(C(=O)c1cc(F)ccc1Oc1nncnc1N1CCC2(C1)CN(C(CCC1OCCO1)C(C)(C)C)C2)C(C)C] | intermediate 10 & intermediate 13 |

### Preparation of intermediate 16

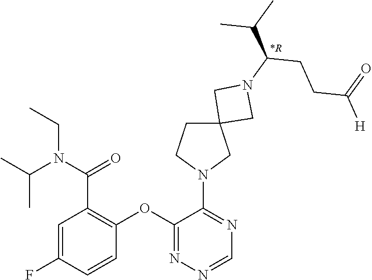 [SMILES: CCN(C(=O)c1cc(F)ccc1Oc1nncnc1N1CCC2(C1)CN([C@H](CCC=O)C(C)C)C2)C(C)C]

A stir bar, intermediate 14a, hydrochloric acid (1 M, 4.4 mL) and acetonitrile (20 mL) were added to a 100 mL round-bottomed flask before the mixture was heated and stirred at 50° C. for 1 h. The mixture was cooled to room temperature, suspended into dichloromethane (40 mL) and adjusted to pH=12 by 10% solution of sodium hydroxide (10 mL). The aqueous layer was extracted with dichloromethane (10 ml×3). The combined organic layers were dried over anhydrous Na₂SO₄, filtered and concentrated under reduced pressure to give intermediate 16 (460 mg, crude) as a light yellow solid.

The following intermediate was synthesized by an analogous method as described above for intermediate 16

| Int. No. | Structure | Starting Materials |
|---|---|---|
| 17 | 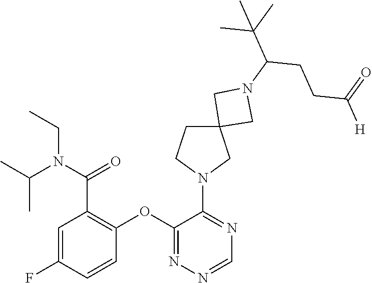 [SMILES: CCN(C(=O)c1cc(F)ccc1Oc1nncnc1N1CCC2(C1)CN(C(CCC=O)C(C)(C)C)C2)C(C)C] | intermediate 5 |

### Preparation of intermediate 18

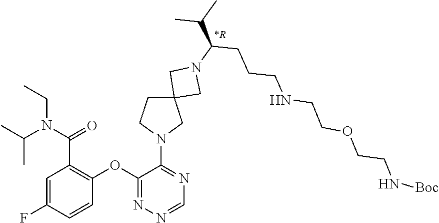 [SMILES: CCN(C(=O)c1cc(F)ccc1Oc1nncnc1N1CCC2(C1)CN([C@H](CCCNCCOCCNC(=O)OC(C)(C)C)C(C)C)C2)C(C)C]

To a mixture of intermediate 16 (200 mg, 0.380 mmol) in MeOH (3 mL) was added tert-butyl (2-(2-aminoethoxy)ethyl)carbamate (78 mg, 0.382 mmol) and acetic acid (50 mg, 0.833 mmol).

The mixture was stirred at room temperature for 30 minutes. Then NaBH₃CN (50 mg, 0.796 mmol) was added to the mixture and the resultant mixture was stirred at room temperature overnight. The reaction mixture was evaporated to remove solvent. The residue was diluted by saturated NaHCO₃ aqueous solution (10 mL), extracted with DCM (10 mL×3). The combined organic layers were washed with brine (10 mL), dried over anhydrous Na₂SO₄, filtered and concentrated under reduced pressure to give the crude, which was purified by FCC (100% dichloromethane to DCM/MeOH=10/1, TLC: dichloromethane: MeOH=10:1, Rf=0.3) to give intermediate 18 (120 mg, purity 65.86%, yield 29.11%) as a yellow solid.

### Preparation of intermediate 19

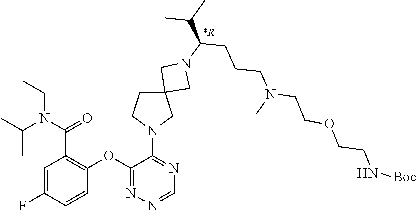 [SMILES: CCN(C(=O)c1cc(F)ccc1Oc1nncnc1N1CCC2(C1)CN([C@H](CCCN(C)CCOCCNC(=O)OC(C)(C)C)C(C)C)C2)C(C)C]

To a mixture of intermediate 18 (110 mg, 0.154 mmol) in MeOH (3 mL) was added formaldehyde (253 mg, 37% solution in H₂O, 3.12 mmol) and acetic acid (20 mg, 0.333 mmol). The mixture was stirred at room temperature for 30 minutes. Then NaBH₃CN (20 mg, 0.318 mmol) was added to the mixture and the resultant mixture was stirred at room temperature overnight. The reaction mixture was evaporated to remove solvent. The residue was diluted by saturated NaHCO₃ aqueous solution (10 mL), extracted with DCM (10 mL×3). The combined organic layers were washed with brine (10 mL), dried over anhydrous Na₂SO₄, filtered and concentrated under reduced pressure to give the crude, which was purified by preparative HPLC (Column: Xtimate C18 100*30 mm*3 μm, Mobile Phase A: water (0.225% FA), Mobile Phase B: acetonitrile, Flow rate: 25 mL/min, gradient condition from 10% B to 40%). The pure fractions were collected and the volatile solvent was evaporated under vacuum to give the residue which was lyophilized to afford intermediate 19 (45 mg, purity 97.89%, yield 39.28%) as a yellow oil.

### Preparation of intermediate 20

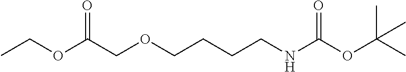 [SMILES: CCOC(=O)COCCCCNC(=O)OC(C)(C)C]

NaH (423 mg, 10.6 mmol) and 10 mL of THF was added into 100 mL of three-necked flask, then tert-butyl (4-hydroxybutyl)carbamate (1 g, 5.28 mmol) in 5 mL of THF was added under argon atmosphere. The mixture was stirred at 0-5° C. for 30 minutes under argon atmosphere. Subsequently ethyl 2-bromoacetate (1.32 g, 7.93 mmol) in 5 mL of THF was added to the above solution. The mixture was stirred at r.t for 16 hours. The reaction mixture was poured into 40 mL of water and extracted with EtOAc (40 mL×2), the combined extracts was washed with brine (50 mL ), dried over Na₂SO₄, filtered and concentrated in vacuum, the residue was purified by column chromatography (PE/EtOAc=10-30%) to afford intermediate 20 (500 mg, yield 31%) as colorless oil.

### Preparation of intermediate 21

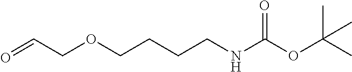 [SMILES: CC(C)(C)OC(=O)NCCCCOCC=O]

To a solution of intermediate 20 (300 mg, 0.926 mmol) in 10 mL toluene was added dropwise DIBAL-H (1.7 mL, 1 M) under N₂ atmosphere below −68° C. After addition, the reaction mixture was stirred below −70° C. for 1 hour. 2 mL of MeOH was added into the reaction mixture bellow −68° C., then the reaction mixture was added into 20 mL of 0.1N HCl and extracted with EtOAc (20 mL×2), the combined extracts was washed with brine (20 mL), dried over Na₂SO₄, filtered and concentrated in vacuum to afford intermediate 21 (170 mg, crude) as light brown oil, which was used for next step directly.

### Preparation of intermediate 22

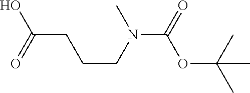

To a solution of 4-(methylamino)butanoic acid (10.0 g, 65.1 mmol) and TEA (26.0 mL, 196 mmol) in MeOH (120 mL) was added Boc₂O (16.0 g, 73.3 mmol) dropwise. The mixture was stirred at rt for 2 days. The mixture was concentrated under reduce pressure to give a residue. The residue was diluted with EtOAc (150 mL), washed with cooled 0.1 N HCl (70 mL×2) and then washed with H₂O (50 mL×2) and brine (50 mL), dried over Na₂SO₄, filtered and concentrated to give desired product (8.90 g, crude) as colorless oil. The crude product was used for next step without further purification.

### Preparation of intermediate 23

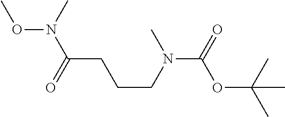 [SMILES: CON(C)C(=O)CCCN(C)C(=O)OC(C)(C)C]

A stir bar, intermediate 22 (8.90 g, 41.0 mmol), N,O-dimethylhydroxylamine hydrochloride (5.00 g, 51.3 mmol), 1H-benzo[d][1,2,3]triazol-1-ol (5.50 g, 40.7 mmol), 4-methylmorpholine (25.0 g, 247 mmol) and CHCl₃ (300 mL) was added into 1 L of round bottomed-flask under N₂ atmosphere. The reaction mixture was cooled to 0° C. and N1-((ethylimino)methylene)-N3,N3-dimethylpropane-1,3-diamine hydrochloride (11.0 g, 57.4 mmol) was added. After that, the reaction mixture was stirred at 27° C. for 16 hours. The reaction mixture was washed with water (200 mL), then 0.1 N HCl (150 mL×2), sat. NaHCO₃ (200 mL×3), brine (200 mL), dried over Na₂SO₄, filtered and concentrated in vacuum and the residue was purified by column chromatography (PE/EtOAc=20%-40%) to afford intermediate 23 (6.30 g, 59% yield) as colorless oil.

### Preparation of intermediate 24

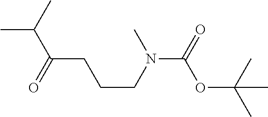 [SMILES: CC(C)C(=O)CCCN(C)C(=O)OC(C)(C)C]

Under N₂ at −70° C., isopropyl lithium (64.8 mL, 45.4 mmol, 0.7M) was added dropwise to a solution of intermediate 23 (4.00 g, 15.4 mmol) in THF (50 mL). The solution was stirred at 70° C. for 2 hours. The mixture was quenched with sat. NH₄Cl (80 mL), extracted with EtOAc (50 mL×3). The combined organic phase was washed with brine (50 mL), dried over Na₂SO₄, filtered and the filtrate was concentrated under reduce pressure to give a crude product. The crude product was purified by FCC (PE: EtOAc=10:1) to afford intermediate 24 (2.20 g, 55% yield) as colorless oil.

### Preparation of intermediate 25, 25a &25b

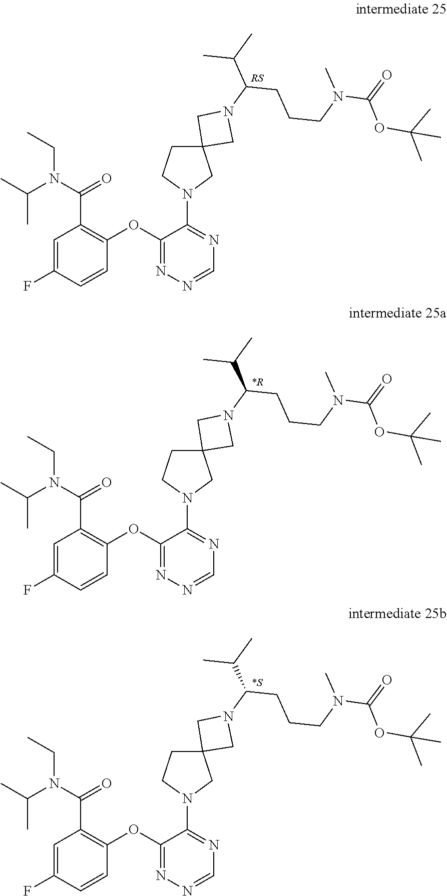

To a solution of intermediate 10 (1.2 g, 2.90 mmol) and intermediate 24 (705 mg, 2.90 mmol) in MeOH (50 mL) was added ZnCl₂ (1.60 g, 11.7 mmol). The mixture was stirred at 80° C. for 2 hours. Then sodium cyanotrihydroborate (1.1 g, 17.5 mmol) was added. The reaction mixture was stirred 80° C. overnight. The mixture was concentrated under reduce pressure to give a residue. The residue was diluted with DCM (100 mL), quenched with sat. NH₄Cl (50 mL). The aqueous was extracted with DCM (50 mL×3). The combined organic phase was washed with brine (50 mL), dried over Na₂SO₄, filtered and the filtrate was concentrated under reduce pressure to give a crude product. The crude product was purified by FCC (DCM: MeOH=10:1) to afford intermediate 25 (1.0 g, 48% yield) as white solid, which was resolved by SFC to give intermediate 25a (400 mg, yield 40%) (Peak 1,Rt=1.326 min) and intermediate 25b (400 mg, 40% yield) (Peak 2, Rt=1.420 min) all as white solid. SFC method: Column: DAICEL CHIRALPAK AD(250 mm*50 mm, 10 um); Mobile phase: A: Supercritical CO₂, B: 0.1% NH₃H₂O IPA, A:B=75:25 at 200 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm.

### Preparation of intermediate 26

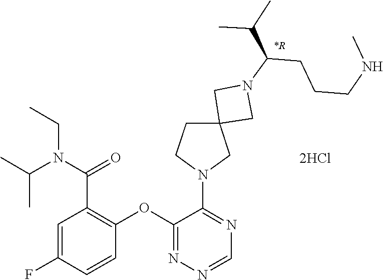

To a solution of intermediate 25a (100 mg, 0.156 mmol) in 2 mL of dioxane was added HCl/dioxane (4 mL, 16 mmol). After addition, the reaction mixture was stirred at r.t for 1 hour. The reaction mixture was concentrated in vacuum to afford intermediate 26 (92 mg, crude) as colorless oil which was used in next step without any purification.

### Preparation of intermediate 27

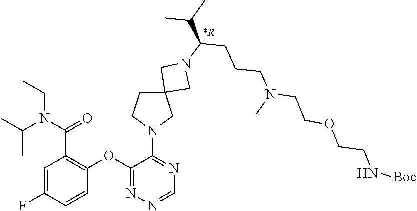 [SMILES: CCN(C(=O)c1cc(F)ccc1Oc1nncnc1N1CCC2(C1)CN([C@H](CCCN(C)CCOCCNC(=O)OC(C)(C)C)C(C)C)C2)C(C)C]

To a solution of intermediate 26 (220 mg, 0.358 mmol) and intermediate 21 (170 mg, 0.735 mmol) in 15 mL of MeOH was added NaOAc (100 mg, 1.22 mmol). After stirred for 15 minutes, NaBH₃CN (50 mg, 0.796 mmol) was added to the mixture. After addition, the reaction mixture was stirred and heated at 35° C. for 16 hours. The reaction mixture was concentrated in vacuum and the residue was diluted with 30 mL of water and basified by 5% NaOH to pH=12, then extracted with DCM (30 ml×3). The combined extracts was washed with brine (30 mL), dried over anhydrous Na₂SO₄, filtered and concentrated in vacuum, the residue was purified by column chromatography (eluent: 100% DCM to DCM/MeOH=20:1 (containing 0.25% NH₃.H₂O)) to afford intermediate 27 (210 mg, 72% yield) as light brown sticky oil.

### Preparation of intermediate 28

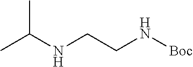

To a solution of tert-butyl (2-aminoethyl)carbamate (5.00 g, 31.2 mmol) and propan-2-one (3.62 g, 62.3 mmol) in MeOH (50 mL) was added acetic acid (3.81 g, 62.4 mmol). The mixture was stirred at room temperature for 0.5 h. Then sodium cyanotrihydroborate (3.93 g, 62.5 mmol) was added. The mixture was stirred at room temperature for 2 h. The mixture was concentrated under reduced pressure, the mixture was diluted with DCM (300 mL). The mixture was washed with NaHCO₃ (100 mL×2) and brine (100 mL×2), dried over Na₂SO₄, filtered and concentrated under reduced pressure. The crude product was purified by flash column chromatography over silica gel (eluent: 100% dichloromethane to dichloromethane: methanol =10:1) to afford intermediate 28 (5.0 g, 79% yield) as yellow oil.

### Preparation of intermediate 29

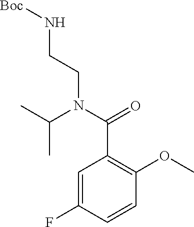 [SMILES: COc1ccc(F)cc1C(=O)N(CCNC(=O)OC(C)(C)C)C(C)C]

To a solution of 5-fluoro-2-methoxybenzoic acid (2.50 g, 14.1 mmol) and intermediate 28 (2.86 g, 14.1 mmol) in DCM (50 mL) was added T3P (18.0 g, 28.3 mmol) and TEA (6.25 mL, 45.0 mmol). The mixture was stirred at room temperature for 1 h. The mixture was diluted with DCM (100 mL), the mixture was washed with NaHCO₃ (50 mL×2) and brine (50 mL×2), dried over Na₂SO₄, filtered and concentrated under reduced pressure to afford intermediate 29 (5.0 g, crude) as yellow oil, which was used for next step without further purification.

### Preparation of intermediate 30

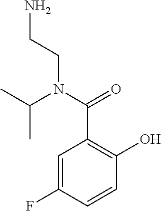 [SMILES: CC(C)N(CCN)C(=O)c1cc(F)ccc1O]

To a solution of intermediate 29 (5.00 g, 14.1 mmol) in DCM (50 mL) was added BBr₃ (4.00 mL, 42.3 mmol) at −78° C. under N₂ atmosphere. The mixture was stirred at −78° C. for 1 h and stirred at room temperature for 12 h. The mixture was quenched with H₂O (20 mL) at 0° C. The mixture extracted with DCM (100 mL), the organic phase was discarded, and the aqueous phase was basified with NaOH (2 M) to pH=11 to afford the solution (3.40 g in solution, theoretical amount), which was used for next step without further purification.

### Preparation of intermediate 31

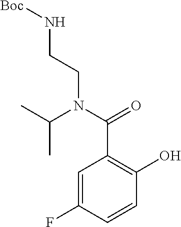 [SMILES: CC(C)N(CCNC(=O)OC(C)(C)C)C(=O)c1cc(F)ccc1O]

To a solution of intermediate 30 (3.40 g, 14.2 mmol, PH=11, in H₂O (50 mL)) was added Boc₂O (3.09 g, 14.2 mmol). The mixture was stirred at room temperature for 1 h. The mixture was diluted with water (50 mL) and extracted with DCM (30 mL×3), dried over Na₂SO₄, filtered and concentrated under reduced pressure. The crude product was purified by flash column chromatography over silica gel (eluent: 100% petroleum ether to petroleum ether: ethyl acetate=1:1) to afford intermediate 31 (900 mg, 90.75% purity, 17.0% yield) as white solid.

### Preparation of intermediate 32

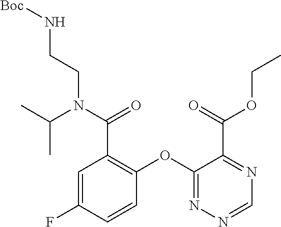 [SMILES: CCOC(=O)c1ncnnc1Oc1ccc(F)cc1C(=O)N(CCNC(=O)OC(C)(C)C)C(C)C]

To a solution of intermediate 31 (900 mg, 2.64 mmol) in DCM (20 mL) was added ethyl 6-oxo-1,6-dihydro-1,2,4-triazine-5-carboxylate (894 mg, 5.29 mmol), TEA (1.30 mL, 13.1 mmol) and PyBrop (2.47 g, 5.30 mmol). The mixture was stirred at 30° C. for 12 h. The mixture was diluted with DCM (30 mL) and washed with water (20 mL×2), dried over Na₂SO₄, filtered and concentrated under reduced pressure. The crude product was purified by flash column chromatography over silica gel (eluent: A: petroleum ether, B: ethyl acetate, 0% B to 50% B in A) to afford intermediate 32 (650 mg, 94.50% purity, 47% yield) as brown solid.

### Preparation of intermediate 33

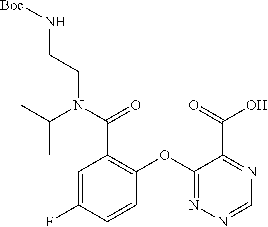 [SMILES: CC(C)N(CCNC(=O)OC(C)(C)C)C(=O)c1cc(F)ccc1Oc1nncnc1C(=O)O]

To a solution of intermediate 21 (340 mg, 0.692 mmol) in THF (6 mL) and H₂O (1 mL) was added LiOH.H₂O (44.0 mg, 1.05 mmol) at 0° C. After addition, the reaction mixture was stirred at 20° C. for 0.5 h. The mixture was adjusted to pH=5 by 1 N HCl and evaporated under reduce pressure to remove solvent. The residue was lyophilized to give intermediate 33 (320 mg, crude) as yellow solid, which was used in the next step without further purification.

### Preparation of intermediate 34

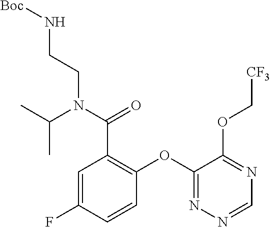 [SMILES: CC(C)N(CCNC(=O)OC(C)(C)C)C(=O)c1cc(F)ccc1Oc1nncnc1OCC(F)(F)F]

4A molecular sieve (1.0 g) was added to a solution of intermediate 33 (320 mg, 0.690 mmol) in 2,2,2-trifluoroethanol (5 mL). The mixture was stirred at 70° C. for 1 h. Then dibromoisocyanuric acid (396 mg, 1.38 mmol) was added. The mixture was stirred at 70° C. for 1 h. The resultant was filtered. The filtrate was concentrated and purified by FCC (eluent: A: petroleum ether, B: ethyl acetate, 0% B to 50% B in A) to give intermediate 34 (170 mg, 45% yield) as yellow oil.

### Preparation of intermediate 35

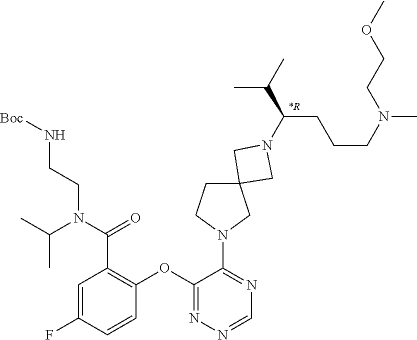 [SMILES: COCCN(C)CCC[C@H](C(C)C)N1CC2(CCN(c3ncnnc3Oc3ccc(F)cc3C(=O)N(CCNC(=O)OC(C)(C)C)C(C)C)C2)C1]

Intermediate 34 (170 mg, 0.329 mmol), (*S)-N-(2-methoxyethyl)-N,5-dimethyl-4-(2,6-diazaspiro[3.4]octan-2-yl)hexan-1-amine hydrochloride (132 mg, 0.395 mmol), DBU (0.15 mL, 1.00 mmol), and CH₃CN (5 mL) were added to a 100 mL round-bottomed flask. The mixture was stirred at 25° C. for 1 hours. The mixture was concentrated under reduced pressure to remove the solvent and diluted with DCM (10 mL), washed with H₂O (10 mL) and brine (10 mL). The organic layer was dried over anhydrous Na₂SO₄, filtered and concentrated under reduced pressure to give crude product, which was purified by preparative HPLC (Column: Phenomenex Gemini-NX 80*40 mm*3 um, Mobile Phase A: water(0.05% NH₃H₂O+10 mM 15 NH₄HCO₃), Mobile Phase B: acetonitrile, Flow rate: 30 mL/min, gradient condition from 44% B to 74%). The pure fractions were collected and the volatile solvent was evaporated under vacuum. The resultant aqueous mixture was extracting with DCM (20 mL×2). The combined organic extracts were dried over Na₂SO₄, filtered, and concentrated to dryness under reduced pressure to afford intermediate 35 (170 mg, yield 72%) as yellow oil.

### Preparation of intermediate 37

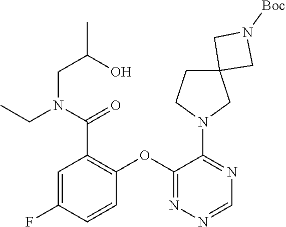 [SMILES: CCN(CC(C)O)C(=O)c1cc(F)ccc1Oc1nncnc1N1CCC2(CN(C(=O)OC(C)(C)C)C2)C1]

To the solution of intermediate 36 (400 mg, 0.871 mmol) in xylene (2 mL) was added 1-(ethylamino)propan-2-ol (449 mg, 4.353 mmol), stirred at 135° C. for 20 h. Then the mixture was concentrated under reduced pressure to afford the residue, which was purified by column chromatography on silica gel (10% MeOH in CH₂Cl₂) to give intermediate 37 as oil (462 mg, 90% purity, 36% yield).

### Preparation of intermediate 38

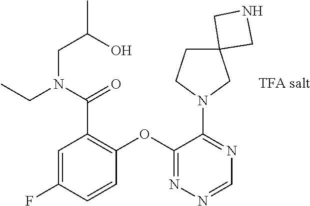 [SMILES: CCN(CC(C)O)C(=O)c1cc(F)ccc1Oc1nncnc1N1CCC2(CNC2)C1]

To the solution of intermediate 37 (200 mg, 0.377 mmol) in methylene chloride (2.1 mL, 1.326 g/mL, 32.786 mmol) was added dropwise TFA (0.7 mL, 1.49 g/mL, 9.147 mmol) at rt, then stirred at rt for 1 h. Then the mixture was concentrated under reduced pressure to afford intermediate 38, which was used directly for next step.

### Preparation of intermediate 39

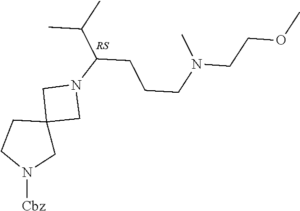 [SMILES: COCCN(C)CCCC(C(C)C)N1CC2(CCN(C(=O)OCc3ccccc3)C2)C1]

To a solution of benzyl 2,6-diazaspiro[3.4]octane-6-carboxylate (5.0 g, 20.3 mmol) in 100 mL of MeOH was added HCl/dioxane (5.1 mL, 20.4 mmol, 4M). The mixture was stirred at 30° C. for 30 minutes. Then AcONa (5.0 g, 61.0 mmol) and 6-((2-methoxyethyl)(methyl)amino)-2-methylhexan-3-one (6.40 g, 31.8 mmol) were added. The reaction mixture was stirred at 30° C. for 15 minutes and NaBH₃CN (2.0 g, 31.8 mmol) added. After addition, the reaction mixture was stirred at 30° C. for 15 hours. The reaction mixture was concentrated, and the residue was diluted with DCM (100 mL), washed with 2N HCl (200 mL×2). The combined aqueous was washed with DCM (100 mL), and then basified by 10% NaOH to pH=12, extracted with DCM (200 mL×3). The combined extracts were washed with brine (200 mL), dried over Na₂SO₄, filtered and concentrated to afford crude compound. The crude product was purified by FCC (DCM: MeOH=10:1) to offer intermediate 39 (2.60 g, 30% yield) as colorless liquid.

### Preparation of intermediate 40

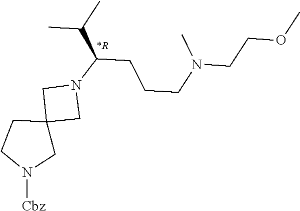 [SMILES: COCCN(C)CCC[C@H](C(C)C)N1CC2(CCN(C(=O)OCc3ccccc3)C2)C1]

The intermediate 39 (2.0 g, 4.63 mmol) was purified by Supercritical Fluid Chromatography (Column: DAICEL CHIRALPAK AY-H(250 mm*30 mm, 5 um), Mobile Phase A: 0.1% NH₃H₂O, Mobile Phase B: EtOH, Flow rate: 100 mL/min, gradient condition from 20% B to 20%). The first faction was collected and evaporated to give intermediate 40 (Rt=2.309 min, 590 mg, 30% yield) as colorless oil.

### Preparation of intermediate 41

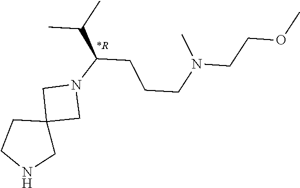 [SMILES: COCCN(C)CCC[C@H](C(C)C)N1CC2(CCNC2)C1]

A stir bar, intermediate 40 (690 mg, 1.45 mmol), 1,1,2-trichloroethane (964 mg, 7.22 mmol) and anhydrous methanol (20 mL) were added to a 100 mL hydrogenated bottle before the mixture was purged with argon for three times, then wet Pd/C (120 mg, 10% purity) was added to the mixture. The resultant mixture was purged with argon and hydrogen for three times, heated and stirred at 40° C. under hydrogen atmosphere (50 psi.) for 4 hours. the mixture was cooled to room temperature and filtered through a pad of Celite®, the filter cake was washed with methanol (10 mL×3). The combined organic layers were concentrated under reduced pressure to give intermediate 41 (120 mg, crude) as a colorless oil.

### Preparation of intermediate 42

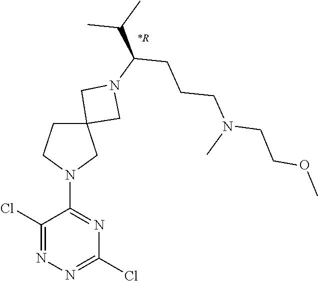 [SMILES: COCCN(C)CCC[C@H](C(C)C)N1CC2(CCN(c3nc(Cl)nnc3Cl)C2)C1]

A stir bar, 3,5,6-trichloro-1,2,4-triazine (1.00 g, 5.42 mmol), intermediate 41 (1.61 g, 5.41 mmol) and anhydrous dichloromethane (20 mL) were added to a 40 mL glass bottle before triethylamine (1.10 g, 10.9 mmol) was added to the mixture dropwise. The resultant mixture was stirred at 25° C. for 8 h. The mixture was concentrated under reduced pressure to give a residue which was suspended into water (40 mL) and extracted with dichloromethane (20 mL×3). The combined organic layers were dried over anhydrous Na₂SO₄, filtered and concentrated under reduced pressure to give the crude which was purified by FCC (100% dichloromethane to dichloromethane: methanol=25:1) to give intermediate 42 (1.20 g, 96.13% purity, 47.8% yield) as a yellow solid.

### Preparation of intermediate 43

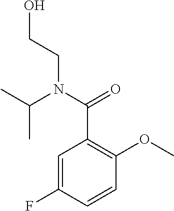 [SMILES: COc1ccc(F)cc1C(=O)N(CCO)C(C)C]

A stir bar, 5-fluoro-2-methoxybenzoic acid (10 g, 58.8 mmol), 2-(isopropylamino)ethanol (12.1 g, 117 mmol), TEA (17.8 g, 176 mmol) and dry dichloromethane (200 mL) were added to a 500 mL round-bottomed flask and then stirred at 0° C. before T3P (56.1 g, 88.2 mmol) was added to the mixture. The resulting mixture was stirred at 25° C. for 12 hours. The reaction mixture was poured into dichloromethane (300 mL) before washed with water (200 mL×3). The organic layer was dried over anhydrous Na₂SO₄, filtered, and concentrated under reduced pressure to give intermediate 43 (11.7 g, 90% purity, 70.2% yield) as yellow oil.

### Preparation of intermediate 44

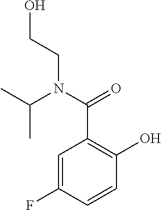 [SMILES: CC(C)N(CCO)C(=O)c1cc(F)ccc1O]

A stir bar, intermediate 43 (11.7 g, 45.8 mmol) and anhydrous dichloromethane (35 mL) were added to a 100 mL three-necked round-bottomed flask before the mixture was cooled to −78° C. under dry ice-ethanol bath, then tribromoborane (9.54 mL, 101 mmol, 2.65 g/mL) was added to the mixture dropwise over 1 h. The resultant mixture was warmed to room temperature gradually and stirred at 25° C. for 1 h. The mixture was cooled to −78° C. under dry ice-ethanol bath and quenched with methanol (30 mL) dropwise. The resultant mixture was added to the saturated solution of sodium bicarbonate (200 mL) slowly. The aqueous layer was extracted with dichloromethane (100 mL×3). The combined organic layers were dried over anhydrous Na₂SO₄, filtered and concentrated under reduced pressure to give the crude which was triturated with petroleum ether/ethyl acetate (50/1, 200 mL) to give intermediate 44 (6.56 g, 90% purity, 53.4% yield) as a light yellow solid.

### Preparation of intermediate 45

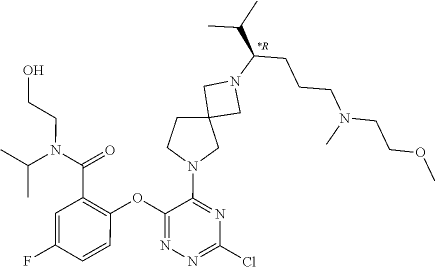

To a solution of intermediate 42 (524 mg, 1.18 mmol) and intermediate 44 (340 mg, 1.41 mmol) in THF (20 mL) was added DBU (340 mg, 2.23 mmol). The reaction mixture was stirred at 25° C. for 12 hours. The reaction mixture was poured into water (30 mL) and extracted with dichloromethane (20 mL×3).The organic layers were dried over anhydrous Na₂SO₄, filtered and concentrated under reduced pressure to give the crude product, which was triturated with petroleum ether/ethyl acetate=6 mL/0.5 mL) and the mixture was filtered, the filter cake was evaporated under vacuum to give intermediate 45 (371 mg, 78.12% purity, 37.89% yield) as yellow solid.

### Preparation of intermediate 46, 46a & 46b

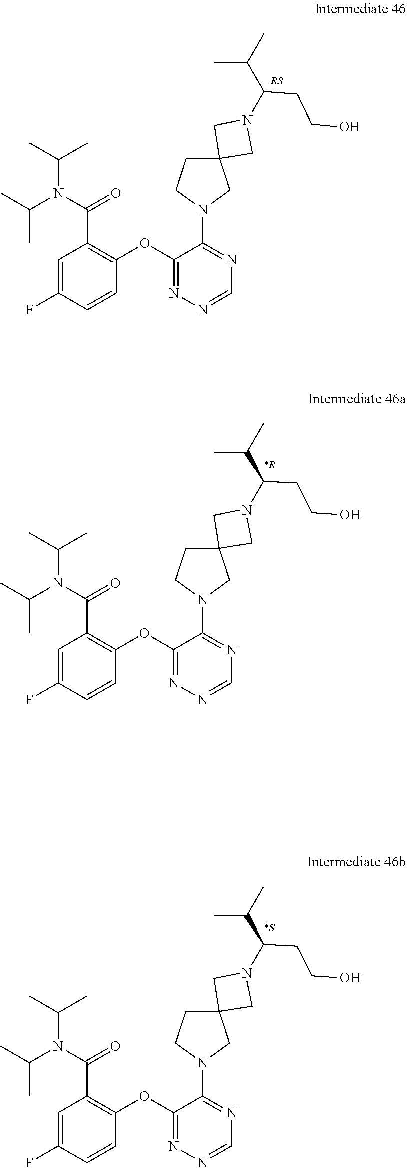 [SMILES: CC(C)C(CCO)N1CC2(CCN(c3ncnnc3Oc3ccc(F)cc3C(=O)N(C(C)C)C(C)C)C2)C1 ; CC(C)[C@@H](CCO)N1CC2(CCN(c3ncnnc3Oc3ccc(F)cc3C(=O)N(C(C)C)C(C)C)C2)C1 ; CC(C)[C@@H](CCO)N1CC2(CCN(c3ncnnc3Oc3ccc(F)cc3C(=O)N(C(C)C)C(C)C)C2)C1]

Intermediate 10 (15 g, 36 mmol) was added to a solution consisting of 1-hydroxy-4-methylpentan-3-one (12.6 g, crude) and AcOH (6.2 mL, 108 mmol), and MeOH (200 mL). The mixture was stirred at room-temperature for 0.5 hour. NaBH₃CN (6.8 g, 108 mmol) was added to the mixture. Then the mixture was stirred at room-temperature for 2 hours. The reaction mixture was adjusted to pH=8 with NH₃ (7 M in MeOH), poured into brine (100 mL) and extracted with ethyl acetate (300 mL×3). The combined organic layer was dried over Na₂SO₄, filtered and concentrated to dryness under reduced pressure to afford the crude product, which was purified by FCC (eluent: petroleum ether: ethyl acetate=1:0 to 0:1, then ethyl acetate: (dichloromethane: methanol=1:1)=1:0 to 1:1) to afford crude intermediate 46 (17 g) as a white solid.

Intermediate 46 (1.6 g) was purified by SFC over DAICEL CHIRALPAK IG 250 mm×30 mm, 10 μm (isocratic elution: MeOH (containing 0.1% of 25% aq. NH₃): supercritical CO₂, 30%: 70% to 30%: 70% (v/v)). The pure fractions were collected, and the volatiles were removed under reduced pressure. The product was suspended in water (10 mL), the mixture frozen using dry ice/acetone, and then lyophilized to dryness to afford intermediate 46a (first fraction, 700 mg, 41%) as a white solid and intermediate 46b (second fraction, 660 mg, 36%) as a white solid.

### Preparation of intermediate 47

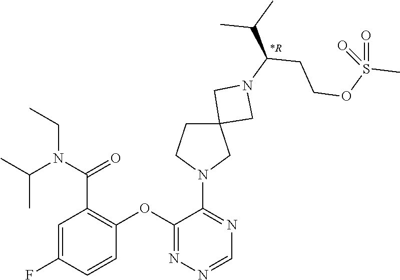 [SMILES: CCN(C(=O)c1cc(F)ccc1Oc1nncnc1N1CCC2(C1)CN([C@H](CCOS(C)(=O)=O)C(C)C)C2)C(C)C]

MsCl (0.169 mL, 1.48 g/mL, 2.182 mmol) was added dropwise to 0° C. (ice/water) solution consisting of intermediate 46a (500 mg, 0.972 mmol), Et₃N (0.27 mL, 1.9 mmol) and dichloromethane (10 mL) under N₂ atmosphere. The resultant mixture was stirred at 0° C. (ice/water) under N₂ for 45 minutes. Quenched with water (5 mL), then extracted with dichloromethane (10 mL×3). The combined organic layers were washed with brine (5 mL), dried over anhydrous Na₂SO₄, filtered and concentrated under reduced pressure to give intermediate 47 (400 mg, yield 62.514%) as a yellow solid, which was used in the next step without further purification.

### Preparation of intermediate 48

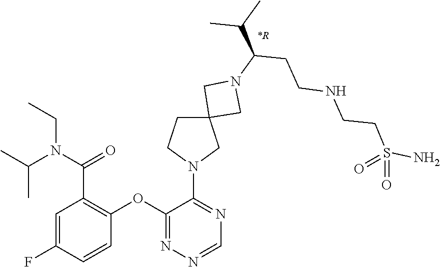 [SMILES: CCN(C(=O)c1cc(F)ccc1Oc1nncnc1N1CCC2(C1)CN([C@H](CCNCCS(N)(=O)=O)C(C)C)C2)C(C)C]

Intermediate 47 (200 mg, 0.304 mmol) and 2-aminoethane-1-sulfonamide (264 mg, 2.126 mmol) were added to THF (25 mL, 0.886 g/mL, 307.182 mmol). The mixture was stirred at 60° C. for 16 hours. The solvent was removed. The residue was purified by flash column (C18, CH₃CN: H₂O from 5:95 to 30:70, HCOOH as buffer) to afford intermediate 48 (40 mg, 19% yield).

### Preparation of intermediate 49

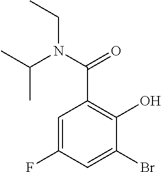 [SMILES: CCN(C(=O)c1cc(F)cc(Br)c1O)C(C)C]

To a solution of N-ethyl-5-fluoro-2-hydroxy-N-isopropylbenzamide (10.0 g, 44.4 mmol) in H₂SO₄ (20 mL) and trifluoroacetic acid (40 mL) was added NBS (8.7 g, 48.9 mmol). The reaction mixture was stirred at 25° C. for 12 hours. The reaction mixture was carefully poured onto 200 g crushed ice. The mixture was extracted with ethyl acetate (100 mL×3). The combined organic layers were dried over anhydrous Na₂SO₄, filtered and concentrated under reduced pressure to give a residue, which was purified by preparative-HPLC (Column: YMC-Triart Prep C18 250*50 mm*10 um, Mobile Phase A: water(0.225% FA), Mobile Phase B: acetonitrile, Flow rate: 100 mL/min, gradient condition from 20% B to 60%). The pure fractions were collected and the solvent was evaporated under vacuum to give intermediate 49(10.0 g, 99.48% purity, 73.7% yield) as a yellow solid.

### Preparation of intermediate 50

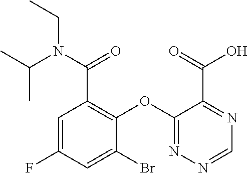 [SMILES: CCN(C(=O)c1cc(F)cc(Br)c1Oc1nncnc1C(=O)O)C(C)C]

To a solution of ethyl 6-chloro-1,2,4-triazine-5-carboxylate (650 mg, 3.47 mmol), intermediate 49 (896 mg, 2.95 mmol) in dry DMF (20 mL) was added potassium carbonate (1.44 g, 10.4 mmol). The reaction mixture was stirred at 25° C. for 12 hours. The mixture was suspended into water (80 mL) and extracted with ethyl acetate (50 mL×3). The combined organic layers were washed with the saturated solution of lithium chloride (50 mL×3). The aqueous layer was lyophilized to dryness to give intermediate 50 (400 mg, crude) as yellow solid, which was used for next step without further purification.

### Preparation of intermediate 51

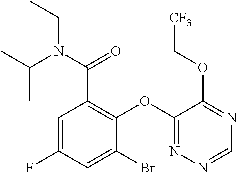 [SMILES: CCN(C(=O)c1cc(F)cc(Br)c1Oc1nncnc1OCC(F)(F)F)C(C)C]

A stir bar, intermediate 50 (300 mg, 0.702 mmol), 4A molecular sieve (1.0 g) and dry 2,2,2-trifluoroethanol (20 mL) were added to a 100 mL round-bottomed flask. The reaction mixture was heated and stirred at 65° C. for 2 hours under argon atmosphere. Then 1,3-dibromo-1,3,5-triazinane-2,4,6-trione (404 mg, 1.41 mmol) was added to the mixture in one portion. The reaction mixture was cooled to room temperature and stirred at 25° C. for another 12 hours. The reaction mixture was filtered and the filter cake was washed with ethyl acetate (50 mL). The filtrate was concentrated under reduced pressure to give a residue, which was purified by flash column chromatography on silica gel (100% petroleum ether to petroleum ether: ethyl acetate =3:1. TLC: petroleum ether: ethyl acetate=3:1, Rf=0.2) to give intermediate 51 (200 mg, 94.47% purity, 55.9% yield) as red oil.

### Preparation of intermediate 52

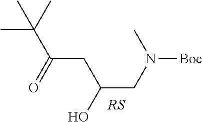 [SMILES: CN(CC(O)CC(=O)C(C)(C)C)C(=O)OC(C)(C)C]

To a solution of 3,3-dimethylbutan-2-one (5.78 g, 57.7 mmol) in anhydrous tetrahydrofuran (80 mL) was added LDA (28.9 mL, 57.8 mmol, 2 M in THF) at −78° C. under N₂ atmosphere. After addition, the reaction mixture was stirred at −78° C. for 1 hour. Then tert-butyl methyl(2-oxoethyl)carbamate (5.0 g, 28.9 mmol) in anhydrous tetrahydrofuran (20 mL) was added dropwise into the reaction mixture and the mixture was stirred at −78° C. for 2 hours. The reaction mixture was quenched with aq. NH₄Cl (20 mL) at −78° C. and water (100 mL) was added, then allowed to warm to 25° C. The mixture was extracted with ethyl acetate (150 mL×3). The combined organic layers were dried over anhydrous Na₂SO₄, filtered and concentrated under reduced pressure to give the crude product, which was purified by preparative-HPLC (Column: Xtimate C18 150*40 mm*5 um, Mobile Phase A: water(0.05%0NH₃H₂O), Mobile Phase B: acetonitrile, Flow rate: 60 mL/min, gradient condition from 35% B to 65%). The pure fractions were collected and the solvent was evaporated under vacuum. The residue was partitioned between acetonitrile (2 mL) and water (10 mL). The mixture was lyophilized to dryness to give intermediate 52 (2.20 g, 100% purity, 27.9% yield) as colourless oil.

### Preparation of intermediate 53a & 53b

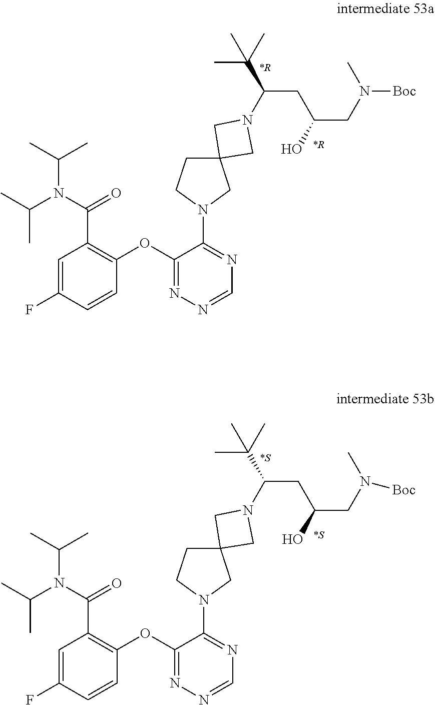 [SMILES: CC(C)N(C(=O)c1cc(F)ccc1Oc1nncnc1N1CCC2(C1)CN([C@H](C[C@@H](O)CN(C)C(=O)OC(C)(C)C)C(C)(C)C)C2)C(C)C ; CC(C)N(C(=O)c1cc(F)ccc1Oc1nncnc1N1CCC2(C1)CN([C@@H](C[C@H](O)CN(C)C(=O)OC(C)(C)C)C(C)(C)C)C2)C(C)C]

ZnCl₂ (449 mg, 3.29 mmol) was added to a solution of intermediate 11 (704 mg, 1.64 mmol) and intermediate 52 (900 mg, 3.29 mmol) in dry methanol (20 mL). The reaction mixture was heated and stirred at 65° C. for 1 hour and then sodium cyanotrihydroborate (207 mg, 3.29 mmol) was added. The reaction mixture was stirred at 65° C. for another 4 days. The reaction mixture was cooled to room temperature and concentrated under reduced pressure to give a residue before the saturated solution of sodium bicarbonate (80 mL) was added. The mixture was extracted with dichloromethane (50 mL×3). The combined organic layers were dried over anhydrous Na₂SO₄, filtered and concentrated under reduced pressure to give the crude product, which was purified by preparative-HPLC (Column: Xtimate C18 150*40 mm*5 um, Mobile Phase A: water(0.05% NH₃H₂O), Mobile Phase B: acetonitrile, Flow rate: 60 mL/min, gradient condition from 50% B to 80%). The pure fractions were collected and the solvent was evaporated under vacuum. The residue was partitioned between acetonitrile (2 mL) and water (10 mL). The mixture was lyophilized to dryness to give the the first fraction (160 mg, 96.85% purity, 13.8% yield) as colorless sticky oil and the second fraction (150 mg, 95.86% purity, 12.8% yield) as colorless sticky oil.

The first fraction (160 mg, 0.233 mmol) was separated by supercritical fluid chromatography (separation condition: DAICEL CHIRALPAK IG (250 mm*30 mm,10 um)); Mobile phase: A: Supercritical CO₂, B: 0.1% NH₃H₂O ETOH, A:B=65:35 at 70 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The pure fraction was collected and the solvent was evaporated under vacuum. The residue was partitioned between acetonitrile (2 mL) and water (10 mL). The solution was lyophilized to dryness to give intermediate 53a (70.0 mg, 100% purity, 43.8% yield) as colorless sticky oil and intermediate 53b (70.0 mg, 97.24% purity, 42.5% yield) as colorless sticky oil.

### Preparation of intermediate 54a & 54b

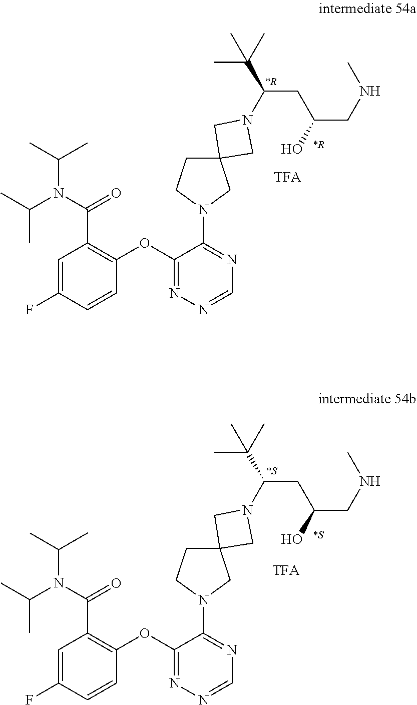 [SMILES: CNC[C@H](O)C[C@@H](N1CC2(CCN(c3ncnnc3Oc3ccc(F)cc3C(=O)N(C(C)C)C(C)C)C2)C1)C(C)(C)C ; CNC[C@@H](O)C[C@H](N1CC2(CCN(c3ncnnc3Oc3ccc(F)cc3C(=O)N(C(C)C)C(C)C)C2)C1)C(C)(C)C]

To a solution of intermediate 53a (70.0 mg, 0.102 mmol) in anhydrous dichloromethane (2 mL) was added trifluoroacetic acid (2 mL). The reaction mixture was stirred at 25° C. for 30 minutes. The reaction mixture was concentrated under reduced pressure to give intermediate 54a (70.0 mg, crude) as yellow oil.

To a solution of intermediate 53b (70.0 mg, 0.102 mmol) in anhydrous dichloromethane (2 mL) was added trifluoroacetic acid (2 mL). The reaction mixture was stirred at 25° C. for 30 minutes. The reaction mixture was concentrated under reduced pressure to give intermediate 54b (70.0 mg, crude) as yellow oil.

### Preparation of Compounds

### Preparation of Compound 1

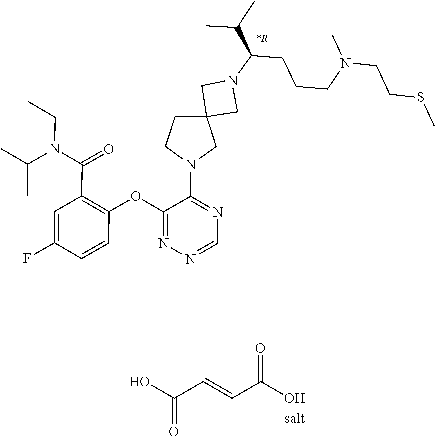

A stir bar, intermediate 16 (120 mg, 0.228 mmol), N-methyl-2-(methylthio)ethanamine hydrochloride (133 mg, 0.939 mmol) and methanol (3 mL) were added to a 40 mL glass bottle, the mixture was heated and stirred at 45° C. for 2 hours before sodium cyanoborohydride (70 mg, 1.11 mmol) was added to the mixture, the mixture was heated and stirred at 45° C. for 8 hours. The mixture was cooled to the room temperature, then the mixture was quenched with water (20 mL), extracted with dichloromethane (30 mL*3), the organic layers were dried over anhydrous Na₂SO₄, filtered and concentrated under reduce pressure to give a crude, which was purified by prep. HPLC (Column: Boston Prime C18 150*30 mm*5 um, Mobile Phase A: water (0.05% NH₃H₂O+10 mM NH₄HCO₃), Mobile Phase B: acetonitrile, Flow rate: 25 mL/min, gradient condition from 70% B to 100% B). The pure fractions were collected, and the solvent was evaporated under vacuum. The residue was partitioned between acetonitrile (2 mL) and water (10 mL). The mixture was lyophilized to dryness to give the product (85 mg, 100% purity, 60.57% yield, free base) as a white solid, which was mixed with fumaric acid (32.2 mg, 0.277 mmol), acetonitrile (12 mL) and water (4 mL) in a 50 mL round-bottomed flask. Then the mixture was concentrated under reduced pressure to afford a residue. The residue was partitioned between acetonitrile (3 mL) and water (10 mL). The solution was lyophilized to dryness to give Compound 1 (81.33 mg, 96.99% purity, 67.40% yield) as a white solid.

### Preparation of Compound 2

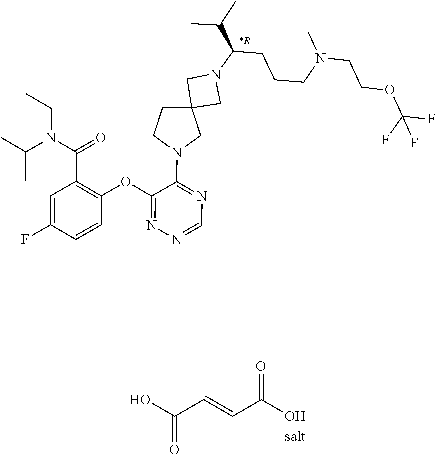

A stir bar, Compound 4 (60 mg, 0.069 mmol, free base form), formaldehyde (55.8 mg, 0.688 mmol), sodium cyanoborohydride (17.2 mg, 0.274 mmol) and methanol (2 mL) were added to a 8 mL glass bottle, the mixture was stirred at 25° C. for 8 hours. The mixture was quenched with water (20 mL), extracted with dichloromethane (20 mL*3), the organic layer were dried over anhydrous Na₂SO₄, filtered and concentrated under reduced pressure to give a crude, which was purified by preparative HPLC (Column: Welch Xtimate C18 150*25 mm*5 um, Mobile Phase A: water (0.2% FA), Mobile Phase B: ACN, Flow rate: 25 mL/min, gradient condition from 15% B to 45%). The pure fractions were adjusted to pH=8 by adding the 10% solution of sodium hydroxide, extracted with dichloromethane (20 mL*3), the organic layers were dried over anhydrous Na₂SO₄, filtered and concentrated under reduced pressure to give a residue. The residue was partitioned between acetonitrile (2 mL) and water (10 mL). The solution was lyophilized to dryness to give the product (10 mg, 100% purity, 22.23% yield) as a colorless oil, which was mixed with fumaric acid (3.6 mg, 0.031 mmol), acetonitrile (12 mL) and water (4 mL) in a 50 mL round-bottomed flask. Then the mixture was concentrated under reduced pressure to afford a residue. The residue was partitioned between acetonitrile (3 mL) and water (10 mL). The solution was lyophilized to dryness to give Compound 2 (8.8 mg, 94.46% purity, 61.34% yield) as a yellow solid.

### Preparation of Compound 3

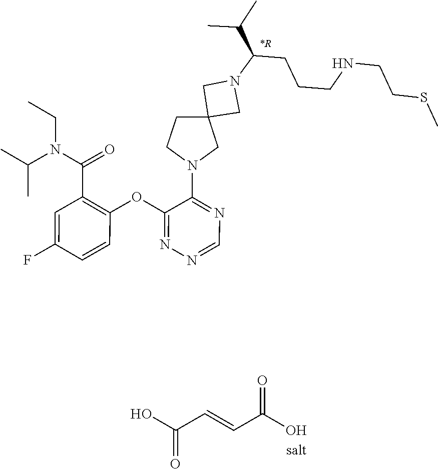

A stir bar, intermediate 16 (120 mg, 0.228 mmol), 2-(methylthio)ethanamine (80 mg, 0.877 mmol), sodium acetate (110 mg, 1.34 mmol) and methanol (3 mL) were added to a 8 mL glass bottle, the mixture was stirred and heated at 60° C. for 2 hours before sodium cyanoborohydride (30 mg, 0.477 mmol) was added to the mixture, the mixture was heated and stirred at 60° C. for 8 hours. The mixture was cooled to the room temperature, then water (30 mL) was poured into the mixture, extracted with dichloromethane (20 mL*3), the organic layers were dried over anhydrous Na₂SO₄, filtered and concentrated under reduced pressure to give a residue, which was purified by preparative HPLC (Column: Boston Prime C18 150*30 mm*5 um, Mobile Phase A: water(0.05% NH₃H₂O+10 mM NH₄HCO₃), Mobile Phase B: acetonitrile, Flow rate: 25 mL/min, gradient condition from 53% B to 83%). The pure fractions were collected, and the solvent was evaporated under vacuum. The residue was partitioned between acetonitrile (2 mL) and water (10 mL). The mixture was lyophilized to dryness to give the product (36 mg, 99.01% purity, 25.99% yield) as a colorless oil, which was mixed with fumaric acid (14.0 mg, 0.121 mmol), acetonitrile (12 mL) and water (4 mL) in a 50 mL round-bottomed flask. Then the mixture was concentrated under reduced pressure to afford a residue. The residue was partitioned between acetonitrile (3 mL) and water (10 mL). The solution was lyophilized to dryness to give Compound 3 (36.17 mg, 91.83% purity, 66.58% yield) as a white solid.

### Preparation of Compound 4

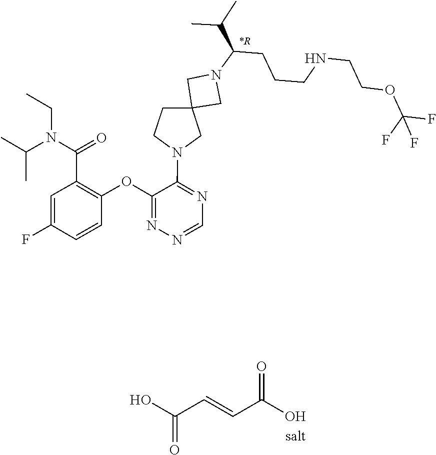

A stir bar, intermediate 16 (200 mg, 0.380 mmol), 2-(trifluoromethoxy)ethanamine hydrochloride (94.3 mg, 0.570 mmol), triethylamine (115 mg, 1.14 mmol) and anhydrous dichloromethane (10 mL) were added to a 40 mL glass bottle before the resultant mixture was stirred at 25° C. for 1 h, then sodium triacetoxyborohydride (161 mg, 0.760 mmol) was added to the mixture. The resultant mixture was stirred at 25° C. for another 1 h. The mixture was diluted into dichloromethane (20 mL) and adjusted to pH=8 by the saturated solution of sodium bicarbonate (10 mL). The aqueous layer was extracted with dichloromethane (10 mL×2). The combined organic layers were dried over anhydrous Na₂SO₄, filtered and concentrated under reduced pressure to give the crude which was purified by preparative HPLC (Column: Boston Green ODS 150*30 mm*5 um, Mobile Phase A: water (0.2% FA), Mobile Phase B: acetonitrile, Flow rate: 35 mL/min, gradient condition from 5% B to 35%). The pure fractions were collected and adjusted to pH=12 by the solution of sodium hydroxide (3 M, 8 mL). The aqueous layer was extracted with dichloromethane (10 mL×2). The combined organic layers were dried over anhydrous Na₂SO₄, filtered and concentrated under reduced pressure to give a residue. The residue was partitioned between acetonitrile (2 mL) and water (10 mL). The solution was lyophilized to dryness to give the product (80.0 mg, 97.84% purity, 32.2% yield) as a colorless solid, which was mixed with fumaric acid (29.0 mg, 0.250 mmol) and acetonitrile (5 mL) in a 50 mL round-bottomed flask before the resultant mixture was stirred at 25° C. for 1 h. The resultant mixture was concentrated under reduced pressure to give a residue. The residue was partitioned between acetonitrile (2 mL) and water (10 mL). The solution was lyophilized to dryness to give Compound 4 (91.20 mg, 89.68% purity, 75.0% yield) as white powder.

### Preparation of Compound 5

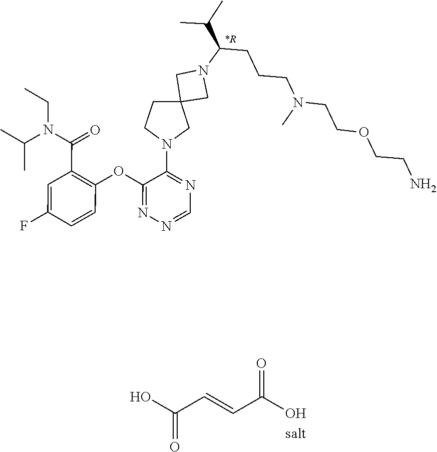

To a mixture intermediate 19 (45 mg, 0.062 mmol) in DCM (3 mL) was added TFA (1 mL) at 0° C. The reaction mixture was stirred at room temperature for 1 hour. The reaction mixture was evaporated under reduce pressure to remove solvent. The residue was diluted by saturated NaHCO₃ aqueous solution (5 mL) and CH₂Cl₂ (5 mL), basified to pH=12 by NaOH (2 N). The resultant mixture was stirred at room temperature for 30 minutes. Then the mixture was separated and the aqueous layer was extracted with dichloromethane (5 mL×2). The combined organic layers were washed with brine (5 mL), dried over anhydrous Na₂SO₄, filtered and concentrated under reduced pressure to give the product (36 mg, yield 92.74%) as a yellow oil, which was mixed with fumaric acid (20 mg, 0.172 mmol), acetonitrile (12 mL) and methanol (4 mL) in a 50 mL round-bottomed flask. Then the mixture was concentrated under reduced pressure to afford a residue. The residue was partitioned between acetonitrile (3 mL) and water (10 mL). The solution was lyophilized to dryness to give Compound 5 (31.06 mg, 91.37% purity, 50.74% yield) as a yellow powder.

### Preparation of Compound 6

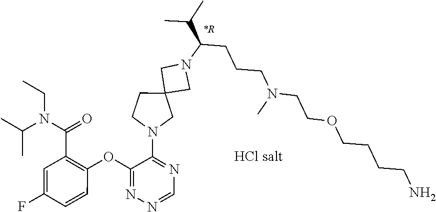

To a solution of intermediate 27 (210 mg, 0.277 mmol) in 3 mL of MeOH was added 5 mL of HCl/dioxane (4M). After addition, the reaction mixture was stirred at 10° C. for 30 minutes. The reaction mixture was concentrated in vacuum, the residue (201 mg) was purified by pre-HPLC (Column Venusil ASB Phenyl 150*30 mm*5 um, Mobile Phase A: water (0.05% HCl), Mobile Phase B: acetonitrile, Flow rate: 30 mL/min, gradient condition from 5% B to 35%). The pure fractions were collected and lyophilized to afford Compound 6 (130 mg, 60% yield) as white solid.

### Preparation of Compound 7

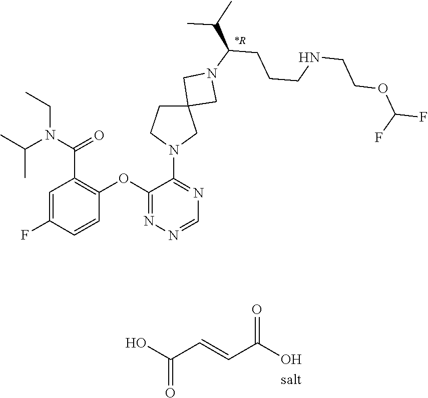

A stir bar, intermediate 16 (200 mg, 0.380 mmol), 2-(difluoromethoxy)ethanamine hydrochloride (112 mg, 0.759 mmol), triethylamine (384 mg, 3.80 mmol) and dichloromethane (2 mL) were added to a 8 mL round-bottomed flask, the mixture was stirred at 25° C. for 2 hours before sodium triacetoxyhydroborate (241 mg, 1.14 mmol) were added to the mixture, the mixture was stirred at 25° C. for 8 hours. The mixture was quenched with water (20 mL), extracted with dichloromethane (30 mL*3), the organic layers were dried over anhydrous Na₂SO₄, filtered and concentrated under reduced pressure to give a crude, which was purified by preparative HPLC (Column: Welch Xtimate C18 150*25 mm*5 um, Mobile Phase A: water(0.2% FA), Mobile Phase B: acetonitrile, Flow rate: 25 mL/min, gradient condition from 5% B to 35%). The pure fractions were collected and the solvent was evaporated under vacuum. The residue was adjusted to pH=8 with the solution of 10% sodium hydroxide (20 mL) extracted with dichloromethane (30 mL*3), the organic layers were dried over anhydrous Na₂SO₄, filtered and concentrated under reduced to give a residue, the residue was partitioned between acetonitrile (2 mL) and water (10 mL). The mixture was lyophilized to dryness to give the product (90 mg, 98.90% purity, 37.70% yield) as colorless oil, which was mixed with fumaric acid (8.6 mg, 0.074 mmol), acetonitrile (12 mL) and water (4 mL) in a 50 mL round-bottomed flask. Then the mixture was concentrated under reduced pressure to afford a residue. The residue was partitioned between acetonitrile (3 mL) and water (10 mL). The solution was lyophilized to dryness to give Compound 7 (21.45 mg, 92.07% purity, 62.52% yield) as a white solid.

### Preparation of Compound 8

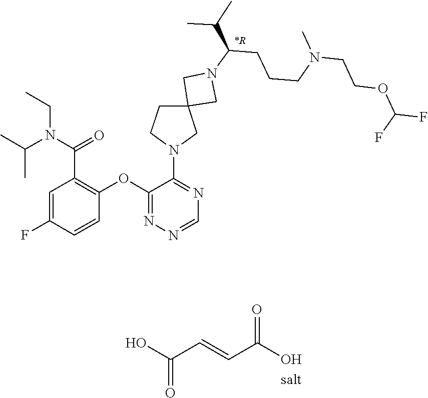

A stir bar, Compound 7 (60 mg, 0.097 mmol, free base form), formaldehyde (78.0 mg, 37% solution in H₂O, 0.961 mmol) and methanol (2 mL) were added to a 50 mL round-bottomed flask, the mixture was stirred at 25° C. for 2 hours before sodium cyanoborohydride (24.2 mg, 0.385 mmol) was added to the mixture, the mixture was stirred at 25° C. for 8 hours. The mixture was quenched with water (20 mL), extracted with dichloromethane (20 mL*3), the organic layers were dried over anhydrous Na₂SO₄, filtered and concentrated under reduced pressure to give a crude, which was purified by preparative HPLC (Column: Welch Xtimate C18 150*25 mm*5 um, Mobile Phase A: water(0.225% FA), Mobile Phase B: ACN, Flow rate: 25 mL/min, gradient condition from 1% B to 30%). The pure fractions were collected and the solvent was evaporated under vacuum to give a residue. The pure fractions were collected and the solvent was evaporated under vacuum. The residue was adjusted to pH=8 with the solution of 10% sodium hydroxide (20 mL) extracted with dichloromethane (30 mL*3), the organic layers were dried over anhydrous Na₂SO₄, filtered and concentrated under reduced to give a residue, the residue was partitioned between acetonitrile (2 mL) and water (10 mL). The mixture was lyophilized to dryness to give the product (40 mg, 95.02% purity, 61.95% yield) as colorless oil, which was mixed with fumaric acid (14.6 mg, 0.126 mmol), acetonitrile (12 mL) and water (4 mL) in a 50 mL round-bottomed flask. Then the mixture was concentrated under reduced pressure to afford a residue. The residue was partitioned between acetonitrile (3 mL) and water (10 mL). The solution was lyophilized to dryness to give Compound 8 (27.02 mg, 99.99% purity, 49.48% yield) as a white solid.

### Preparation of Compound 9

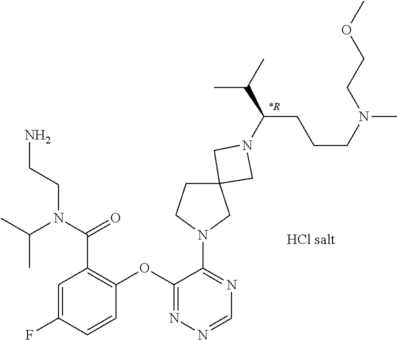 [SMILES: COCCN(C)CCC[C@H](C(C)C)N1CC2(CCN(c3ncnnc3Oc3ccc(F)cc3C(=O)N(CCN)C(C)C)C2)C1]

HCl/dioxane (1 mL, 4 M) was added to a solution of intermediate 35 (150 mg, 0.210 mmol) in DCM (3 mL), the mixture was stirred at 25° C. for 0.5 hour. The solution was concentrated to get the crude product, which was partitioned between acetonitrile (3 mL) and water (10 mL). The solution was lyophilized to dryness to give Compound 9 (99.54 mg, 98.85% purity, 65% yield) as yellow solid.

The following compounds were synthesized by an analogous method as described above for Compound 9

| Compound No. | Structure | Starting Materials |
|---|---|---|
| Compound 10 | 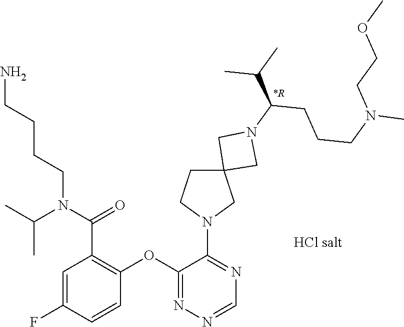 [SMILES: COCCN(C)CCC[C@H](C(C)C)N1CC2(CCN(c3ncnnc3Oc3ccc(F)cc3C(=O)N(CCCCN)C(C)C)C2)C1] | tert-butyl (4- aminobutyl)carbamate |
| Compound 15 | 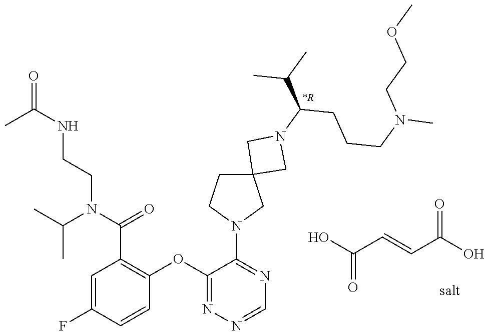 [SMILES: COCCN(C)CCC[C@H](C(C)C)N1CC2(CCN(c3ncnnc3Oc3ccc(F)cc3C(=O)N(CCNC(C)=O)C(C)C)C2)C1 ; O=C(O)/C=C/C(=O)O] | N-(2- Acetamidoethyl)-5- fluoro-N-isopropyl-2- methoxybenzamide |

### Preparation of Compound 11

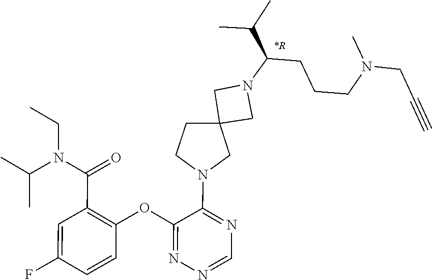

Intermediate 16 (100 mg, 0.190 mmol) was added to a solution consisting of N-methylpropargylamine (26.2 mg, 0.380 mmol), MeOH (5 mL), and HOAc (34.2 mg, 0.570 mmol). The mixture was stirred at room temperature for 15 mins. Then NaBH₃CN (10.7 mg, 0.171 mmol) was added to the solution and stirred for another 45 mins. The mixture was purified by preparative HPLC using a Boston Prime C18 150*30 mm*5 um (eluent: 5500 to 85% MeCN and water (0.05% NH₃H₂O+10 mM NH₄HCO₃) to afford pure product. The product was suspended in water (10 mL), frozen using dry ice/EtOH, and then lyophilized to dryness to afford the title compound (18.52 mg, 16% yield) as a white solid.

### Preparation of Compound 12

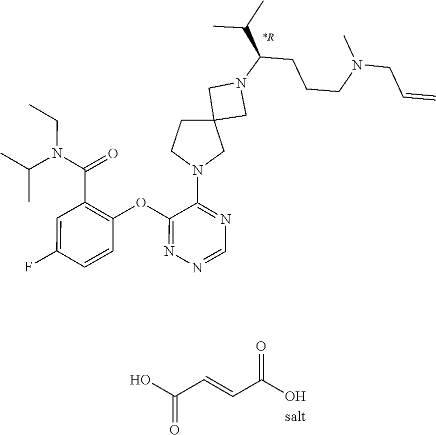

Intermediate 16 (100 mg, 0.190 mmol) was added to a solution consisting of N-allylmethylamine (27 mg, 0.38 mmol), MeOH (1 mL), and HOAc (34.2 mg, 0.570 mmol). Then the mixture was stirred at room temperature for 15 mins. Then NaBH₃CN (10.7 mg, 0.171 mmol) was added to the solution and stirred for another 45 mins. The mixture was purified by preparative HPLC using a Boston Prime C18 150*30 mm*5 um (eluent: 50% to 80% MeCN and water (0.05% NH₃H₂O) to afford pure product. The product was suspended in water (10 mL), frozen using dry ice/EtOH, and then lyophilized to dryness to afford the product (55.8 mg, 51% yield) as a brown oil, which was mixed with fumaric acid (22.3 mg, 0.192 mmol) in MeCN (2 mL), and H₂O (2 mL). Then the mixture was concentrated under reduced pressure to afford a residue. The residue was partitioned between acetonitrile (1 mL) and water (3 mL). The solution was lyophilized to dryness to give Compound 12 (28.19 mg, 35% yield) as a white solid.

### Preparation of Compound 13a & 13b

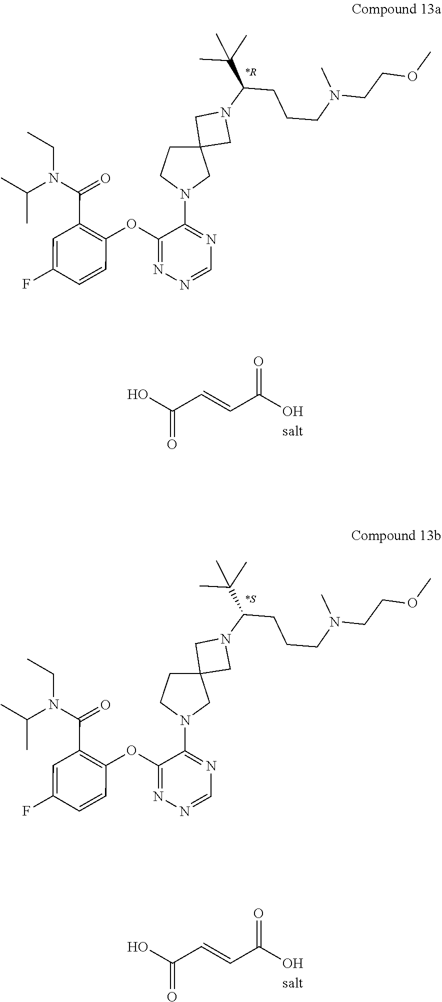

A stir bar, intermediate 17 (190 mg, 0.351 mmol), 2-methoxy-N-methylethanamine (157 mg, 1.76 mmol), and anhydrous dichloromethane (10 mL) were added to a 40 mL glass bottle before the resultant mixture was stirred at 25° C. for 1 h, then sodium triacetoxyborohydride (150 mg, 0.708 mmol) was added to the mixture. The resultant mixture was stirred at 25° C. for another 8 h. The mixture was quenched with the saturated solution of sodium bicarbonate (60 mL) and extracted with dichloromethane (30 mL×2). The combined organic layers were dried over anhydrous Na₂SO₄, filtered and concentrated under reduced pressure to give the crude which was purified by preparative HPLC (Column: Boston Prime C18 150*30 mm*5 um, Mobile Phase A: water (0.05% NH₃H₂O), Mobile Phase B: acetonitrile, Flow rate: 30 mL/min, gradient condition from 55% B to 85%). The pure fractions were collected and the solvent was evaporated under vacuum to give a residue. The residue was partitioned between acetonitrile (2 mL) and water (10 mL). The solution was lyophilized to dryness to give the product (150 mg, 97.102% purity, 67.5% yield) as light yellow oil.

Compound 13a (150 mg, 0.244 mmol) was separated by SFC (DAICEL CHIRAL TECHNOLOGIES (CHINA) CO., LTD.) (Separation condition: CHIRALPAK IG-3(IG30CD-WE016) (0.46 cm I.D.×15 cm L); Mobile phase: ACN/DEA=100/0.1(V/V), at 1.0 mL/min; Column Temp: 35; Nozzle Pressure: 100 Bar; Nozzle Temp: 60; Evaporator Temp: 20; Trimmer Temp: 25; Wavelength: UV 254 nm). The fractions of first peak were collected and the solvent was evaporated under vacuum to give a residue. The residue was partitioned between acetonitrile (2 mL) and water (10 mL). The solution was lyophilized to dryness to give light yellow oil (60.0 mg, 84.967% purity, 34.0% yield) which was further purified by preparative HPLC (Column: Boston Prime C18 150*30 mm*5 um, Mobile Phase A: water (0.05% NH₃H₂O+10 mM NH₄HCO₃), Mobile Phase B: acetonitrile, Flow rate: 25 mL/min, gradient condition from 65% B to 95%). The pure fractions were collected and the solvent was evaporated under vacuum to give a residue. The residue was partitioned between acetonitrile (2 mL) and water (10 mL). The solution was lyophilized to dryness to give Compound Ia (42.2 mg, 99.047% purity, 69.7% yield) as yellow oil which was mixed with fumaric acid (16.0 mg, 0.138 mmol) and acetonitrile (5 mL) in a 50 mL round-bottomed flask before the resultant mixture was stirred at 25° C. for 1 h. The resultant mixture was concentrated under reduced pressure to give a residue. The residue was partitioned between acetonitrile (2 mL) and water (10 mL). The solution was lyophilized to dryness to give Compound 13a (46.78 mg, 97.40% purity, 78.3% yield) as white powder.

The fractions of second peak were collected and the solvent was evaporated under vacuum to give a residue. The residue was partitioned between acetonitrile (2 mL) and water (10 mL). The solution was lyophilized to dryness to give a crude material (60.0 mg, 94.954% purity, 38.0% yield) as light yellow oil which was purified by preparative HPLC (Column: Boston Prime C18 150*30 mm*5 um, Mobile Phase A: water (0.05% NH₃H₂O+10 mM NH₄HCO₃), Mobile Phase B: acetonitrile, Flow rate: 25 mL/min, gradient condition from 65% B to 95%). The pure fractions were collected and the solvent was evaporated under vacuum to give a residue. The residue was partitioned between acetonitrile (2 mL) and water (10 mL). The solution was lyophilized to dryness to give Compound 1b (47.5 mg, 100% purity, 79.2% yield) as yellow oil, which was mixed with fumaric acid (18.0 mg, 0.155 mmol) and acetonitrile (5 mL) in a 50 mL round-bottomed flask before the resultant mixture was stirred at 25° C. for 1 h. The resultant mixture was concentrated under reduced pressure to give a residue. The residue was partitioned between acetonitrile (2 mL) and water (10 mL). The solution was lyophilized to dryness to give Compound 13b (43.36 mg, 97.35% purity, 64.5% yield) as white powder.

### Preparation of Compound 14

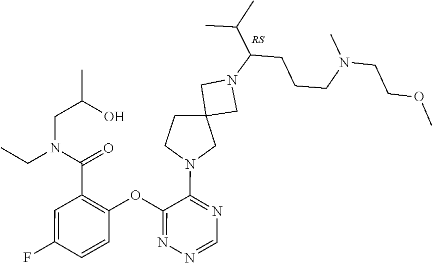

To the solution of intermediate 38 (crude from previous step, 200 mg, 0.465 mmol) in MeOH (2 mL, 0.791 g/mL, 49.372 mmol) was added 6-((2-methoxyethyl)(methyl)amino)-2-methylhexan-3-one (140.291 mg, 0.697 mmol), followed by sodium acetate (114.3 mg, 1.394 mmol), stirred at rt for 10 min, then sodium cyanoborohydride (87.6 mg, 1.394 mmol) was added, stirred at rt for 16 h. Then, the reaction mixture was sent to HPLC purification (preparation method: waters X-bridge C18 (5 μm 19 * 150 mm), Mobile Phase A: water (0.1% ammonium bicarbonate), Mobile Phase B: acetonitrile, UV: 214 nm, Flow rate: 15 mL/min, Gradient: 20-70% (% B) to give Compound 14 (48 mg, 98% purity, 16.4% yield).

### Preparation of Compound 16, 17 & 18

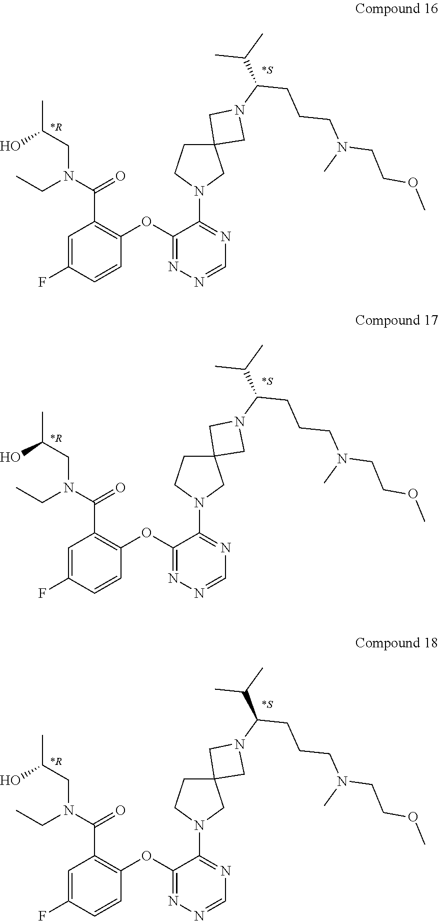 [SMILES: CCN(C[C@@H](C)O)C(=O)c1cc(F)ccc1Oc1nncnc1N1CCC2(C1)CN([C@@H](CCCN(C)CCOC)C(C)C)C2 ; CCN(C[C@H](C)O)C(=O)c1cc(F)ccc1Oc1nncnc1N1CCC2(C1)CN([C@@H](CCCN(C)CCOC)C(C)C)C2 ; CCN(C[C@@H](C)O)C(=O)c1cc(F)ccc1Oc1nncnc1N1CCC2(C1)CN([C@H](CCCN(C)CCOC)C(C)C)C2]

The mixture of Compound 14 (45 mg, 0.073 mmol) was separated by SFC (separation condition: DAICEL CHIRALPAK IG (250 mm*30 mm,10 um); Mobile phase: A: Supercritical C02, B: 0.1% NH₃.H₂O IPA, A:B=55:45 at 70 mL/min). Three fractions were obtained. The first fraction was collected and the solvent was evaporated under vacuum. The residue was partitioned between acetonitrile (2 mL) and water (10 mL). The solution was lyophilized to dryness (20 mg, 94% purity on LCMS) as colorless oil, which was further separated by SFC (separation condition: DAICEL CHIRALPAK IG (250 mm*30 mm,10 um); Mobile phase: A: Supercritical CO₂, B: 0.1% NH₃H₂O EtOH A:B=75:25 at 60 mL/min). The pure fractions of first peak were collected and the solvent was evaporated under vacuum. The residue was partitioned between acetonitrile (2 mL) and water (10 mL). The solution was lyophilized to dryness to give Compound 18 (3.51 mg, 87.38% purity, 15.34% yield) as colorless oil.

The second fraction was collected and the solvent was evaporated under vacuum. The residue was suspended in water (10 mL), the mixture frozen using dry ice/ethanol, and then lyophilized to dryness to afford Compound 16 (2.0 mg, 81.66% purity, 3.63% yield) as colorless oil.

The third fraction was collected and the solvent was evaporated under vacuum. The residue was suspended in water (10 mL), the mixture frozen using dry ice/ethanol, and then lyophilized to dryness to afford Compound 17 (2.79 mg, 79.48% purity, 4.93% yield) as colorless oil.

### Preparation of Compound 19

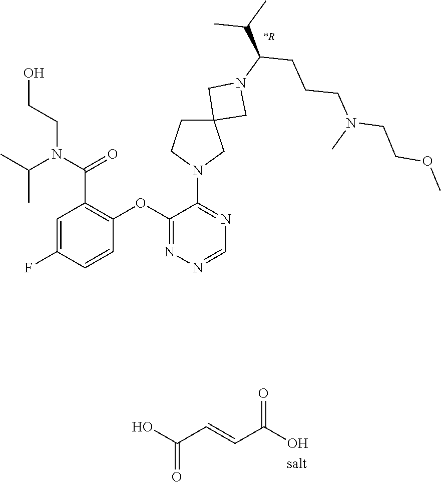 [SMILES: COCCN(C)CCC[C@H](C(C)C)N1CC2(CCN(c3ncnnc3Oc3ccc(F)cc3C(=O)N(CCO)C(C)C)C2)C1 ; O=C(O)/C=C/C(=O)O]

A stir bar, intermediate 45 (270 mg, 0.42 mmol), wet palladium on activated carbon (100 mg, w/w %=10%, containing 50% water) and anhydrous methanol (20 mL) were added to a hydrogenated bottle, then triethylamine (126 mg, 1.25 mmol) was added to the mixture. The suspension was degassed under vacuum and purged with N₂ atmosphere for three times, and then purged with hydrogen for three times. The resulting mixture was stirred under hydrogen (15 psi) at 25° C. for 12 hours. The reaction mixture was filtered through a pad of Celite® and the filter cake was washed with methanol (20 mL×3). The combined filtrates were concentrated under reduced pressure to give the crude product which was dissolved with dichloromethane (50 mL). The organic layer was washed with 10% aqueous NaOH (20 mL), water (20 mL) and brine (20 mL), dried over Na₂SO₄, filtered and concentrated under reduced pressure to give the product (99.1 mg, 95.60% purity, 37.1% yield) as a yellow solid, which was mixed with fumaric acid (37.4 mg, 0.32 mmol) and MeCN (2 mL) in a 50 mL round-bottomed flask. The reaction mixture was stirred at 25° C. for 30 minutes. The reaction mixture was concentrated under reduced pressure to give a residue, which was partitioned between acetonitrile (1 mL) and water (3 mL). The mixture was lyophilized to dryness to give Compound 19 (79.37 mg, 88.30% purity, 51.4% yield) as a yellow solid.

### Preparation of Compound 20

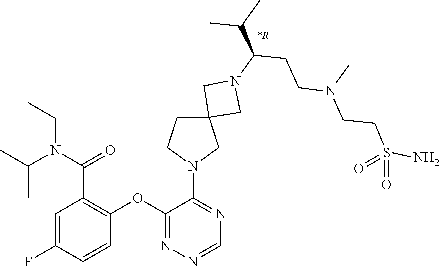 [SMILES: CCN(C(=O)c1cc(F)ccc1Oc1nncnc1N1CCC2(C1)CN([C@H](CCN(C)CCS(N)(=O)=O)C(C)C)C2)C(C)C]

Intermediate 48 (60 mg, 0.0967 mmol) and paraformaldehyde (2.902 mg, 0.0967 mmol) were added to MeOH (4 mL, 0.791 g/mL, 98.745 mmol). The mixture was stirred at RT for 0.5 hour. NaBH₃CN (6.074 mg, 0.0967 mmol) was added. The mixture was stirred for 16 hours. The mixture was purified by flash column (C18, CH₃CN: H₂O from 5:95 to 30:70, HCOOH as buffer) to afford Compound 20 (4 mg, 5.2% yield).

### Preparation of Compound 21

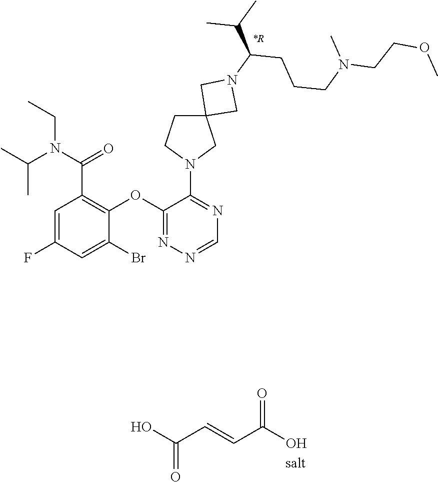 [SMILES: CCN(C(=O)c1cc(F)cc(Br)c1Oc1nncnc1N1CCC2(C1)CN([C@H](CCCN(C)CCOC)C(C)C)C2)C(C)C ; O=C(O)/C=C/C(=O)O]

To a solution of intermediate 51 (200 mg, 0.416 mmol) and intermediate 41 (185 mg, 0.622 mmol) in dry acetonitrile (10 mL) was added DBU (190 mg, 1.25 mmol). The reaction mixture was stirred at 25° C. for 12 hours. The reaction mixture was poured into water (50 mL) and extracted with dichloromethane (30 mL×3). The combined organic layers were dried over anhydrous Na₂SO₄, filtered and concentrated under reduced pressure to give a residue, which was purified by preparative-HPLC (Column: Boston Green ODS 150*30 mm*5 um, Mobile Phase A: water(0.225% FA), Mobile Phase B: acetonitrile, Flow rate: 35 mL/min, gradient condition from 10% B to 40%). The pure fractions were collected and the solvent was evaporated under vacuum. The residue was partitioned between acetonitrile (2 mL) and water (10 mL). The mixture was lyophilized to dryness to give the desired compound (150 mg, 98.35% purity, 52.3% yield)

The product (89.2 mg, 0.131 mmol), fumaric acid (30.51 mg, 0.263 mmol) and MeCN (2 mL) were added a 50 mL round-bottomed flask. The reaction mixture was stirred at 25° C. for 30 minutes. The reaction mixture was concentrated under reduced pressure to give a residue, which was partitioned between acetonitrile (1 mL) and water (3 mL). The mixture was lyophilized to dryness to give Compound 21 (78.99 mg, 98.86% purity, 65.2% yield) as a yellow solid.

### Preparation of Compound 22

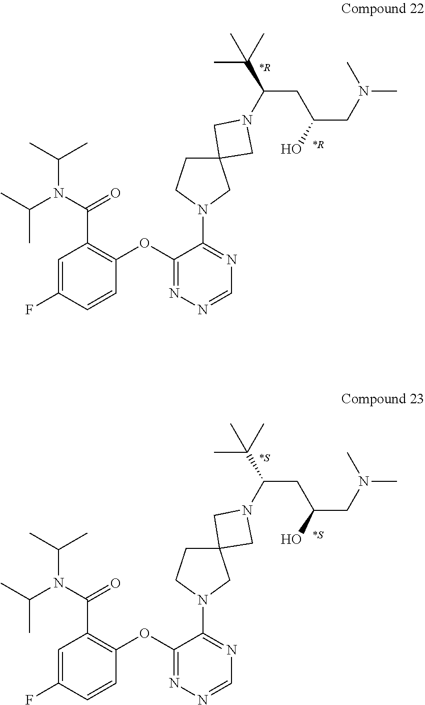

Triethylamine (50.6 mg, 0.500 mmol) was added to a solution of intermediate 54a (70.0 mg, 0.100 mmol) in dry dichloromethane (5 mL). Then formaldehyde aqueous (40.6 mg, 0.500 mmol) was added. The reaction mixture was stirred at 25° C. for 30 minutes before sodium triacetoxyborohydride (42.4 mg, 0.200 mmol) was added. The reaction mixture was stirred at 25° C. for another 12 hours. The reaction mixture was diluted with dichloromethane (50 mL) and the saturated solution of sodium bicarbonate (50 mL) was added, the mixture was extracted with dichloromethane (30 mL×3). The combined organic layers were dried over anhydrous Na₂SO₄, filtered and concentrated under reduced pressure to give a residue, which was purified by preparative-HPLC (Column: Boston Prime C18 150*25 mm*5 um, Mobile Phase A: water(0.05% NH₃H₂O), Mobile Phase B: acetonitrile, Flow rate: 35 mL/min, gradient condition from 51% B to 81%). The pure fractions were collected and the solvent was evaporated under vacuum. The residue was partitioned between acetonitrile (2 mL) and water (10 mL). The mixture was lyophilized to dryness to give Compound 22 (30.79 mg, 98.83% purity, 50.7% yield) as a white powder.

Triethylamine (50.6 mg, 0.500 mmol) was added to a solution of intermediate 54b (70.0 mg, 0.100 mmol) in dry dichloromethane (5 mL). Then formaldehyde aqueous (40.6 mg, 0.500 mmol) was added. The reaction mixture was stirred at 25° C. for 30 minutes before sodium triacetoxyborohydride (42.4 mg, 0.200 mmol) was added. The reaction mixture was stirred at 25° C. for another 12 hours. The reaction mixture was diluted with dichloromethane (50 mL) and the saturated solution of sodium bicarbonate (50 mL) was added, the mixture was extracted with dichloromethane (30 mL×3). The combined organic layers were dried over anhydrous Na₂SO₄, filtered and concentrated under reduced pressure to give a residue, which was purified by preparative-HPLC (Column: Boston Prime C18 150*30 mm*5 um, Mobile Phase A: water(0.05% NH₃H₂O), Mobile Phase B: acetonitrile, Flow rate: 35 mL/min, gradient condition from 50% B to 80%). The pure fractions were collected and the solvent was evaporated under vacuum. The residue was partitioned between acetonitrile (2 mL) and water (10 mL). The mixture was lyophilized to dryness to give Compound 23 (29.02 mg, 99.54% purity, 48.1% yield) as a white powder.

The following compounds were synthesized by an analogous method as described above for Compound 22 & 23

| Compound No. | Structure | Starting Materials |
|---|---|---|
| Compound 24 | 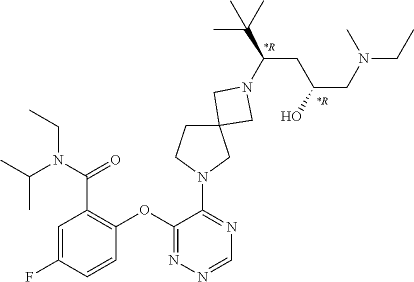 [SMILES: CCN(C)C[C@H](O)C[C@@H](N1CC2(CCN(c3ncnnc3Oc3ccc(F)cc3C(=O)N(CC)C(C)C)C2)C1)C(C)(C)C] | intermediate 10 & intermediate 52 |
| Compound 25 | 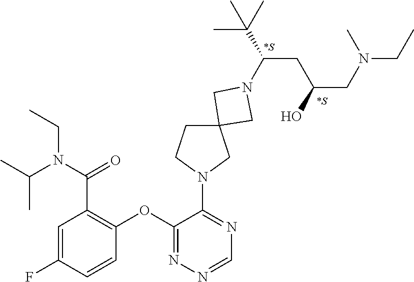 [SMILES: CCN(C)C[C@@H](O)C[C@H](N1CC2(CCN(c3ncnnc3Oc3ccc(F)cc3C(=O)N(CC)C(C)C)C2)C1)C(C)(C)C] | intermediate 10 & intermediate 52 |

LCMS (Liquid chromatography/Mass spectrometry)
General procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻(deprotonated molecule). In case the compound was not directly ionizable the type of adduct is specified (i.e. [M+NH₄]⁺, [M⁺HCOO]⁻, etc., ). For molecules with multiple isotopic patterns (Br, Cl.), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector.

TABLE 1a
LCMS Method codes (Flow expressed in mL/min; column
temperature (T) in ° C.; Run time in minutes).

| | | | | | Flow — | |
|---|---|---|---|---|---|---|
| | | | | | (ml/mn) | |
| | | | | | — | Run |
| Method | | | Mobile | | Column | time |
| code | Instrument | Column | phase | Gradient | T (° C.) | (min) |
| 1 | Agilent | Waters | mobile phase | 100% A was held for 1 min, | 0.8 | 10 |
| | | XBridge | A: H₂O with | A gradient from 100% A to | — | |
| | | C18 | 0.04% TFA; | 40% A is applied in 4 min, | 50 | |
| | | (2.0 × 50 mm, | mobile phase | and 40% A down to 15% A | | |
| | | 5 uM) | B: ACN with | in 2.5 min. And then return | | |
| | | | 0.02% TFA | to 100% A in 2 min and | | |
| | | | | held for 0.5 min. The post | | |
| | | | | time is 0.5 min. | | |
| 2 | Agilent | Waters | mobile phase | First, 90% A was held for | 0.8 | 10 |
| | | XBridge | A: H₂O with | 0.8 min. Then a gradient | — | |
| | | C18 | 0.04% TFA; | was applied to 20% A and | 50 | |
| | | (2.0 × 50 mm, | mobile phase | 80% B in 3.7 min and held | | |
| | | 5 um) | B: ACN with | for 3 min. And then return | | |
| | | | 0.02% TFA | to 90% A in 2 min and held | | |
| | | | | for 0.5 min. The post time | | |
| | | | | is 0.5 min. | | |
| 3 | Agilent | Waters | mobile phase | First, 100% A was held for | 0.8 | 10 |
| | | XBridge | A: H₂O with | 1 min. Then a gradient was | — | |
| | | Shield RP18 | 0.05% | applied to 40% A and 60% | 40 | |
| | | (2.1 × 50 mm, | ammonia; | B in 4 min and then to 5% | | |
| | | 5 um) | mobile phase | A and 95% B in 2.5 min. | | |
| | | | B: ACN | Finally return to 100% A in | | |
| | | | | 2 min and held for 0.5 min. | | |
| | | | | Post Time is 0.5 min. | | |
| 4 | Agilent: | Waters: | A: 0.1% FA | Gradient start from 5% of | 1.2 | 3.5 |
| | 1260 | Sunfire C18 | solution in | B increase to 95% within | — | |
| | Infinity and | (2.5 μm, | water | 2.5 min and keep at 95% | 50 | |
| | 6120 | 3.0 × 30 mm) | B: CH₃CN | till 3.5 min | | |
| | Quadrupole | | | | | |
| | LC/MS | | | | | |

“TFA” means trifluoroacetic acid;
“FA” means formic acid

TABLE 1b
LCMS and melting point data. Co. No. means compound
number; Rₜ means retention time in min.

| Co. No. | Rt | [M + H]⁺ | LCMS |
|---|---|---|---|
| 1 | 2.071 | 616.3 | 3 |
| 2 | 2.220 | 654.3 | 3 |
| 3 | 2.060 | 602.4 | 3 |
| 4 | 2.171 | 640.3 | 3 |
| 5 | 2.863 | 629.4 | 2 |
| 6 | 2.790 | 657.5 | 2 |
| 7 | 3.022 | 622.3 | 2 |
| 8 | 5.374 | 636.4 | 1 |
| 9 | 2.572 | 615.8 | 2 |
| 10 | 2.633 | 643.4 | 2 |
| 11 | 1.894 | 580.3 | 3 |
| 12 | 1.943 | 582.3 | 3 |
| 13a | 2.062 | 614.4 | 3 |
| 13b | 2.066 | 614.4 | 3 |
| 14 | 1.35 | 616.3 | 4 |
| 15 | 2.752 | 657.4 | 2 |
| 16 | 2.721 | 616.3 | 2 |
| 17 | 2.712 | 616.3 | 2 |
| 18 | 2.726 | 616.3 | 2 |
| 19 | 2.799 | 616.3 | 2 |
| 20 | 1.47 | 635.2 | 4 |
| 21 | 2.202 | 680.3 | 3 |
| 22 | 2.377 | 600.3 | 3 |
| 23 | 2.382 | 600.4 | 3 |
| 24 | 2.307 | 600.3 | 3 |
| 25 | 2.288 | 600.3 | 3 |

### Pharmacological Part

1) Menin/MLL homogenous time-resolved fluorescence (HTRF) assay

To an untreated, white 384-well microtiter plate was added 40 nL 200X test compound in DMSO and 4 μL 2X terbium chelate-labeled menin (vide infra for preparation) in assay buffer (40 mM Tris-HCl, pH 7.5, 50 mM NaCl, 1 mM DTT (dithiothreitol) and 0.05% Pluronic F-127). After incubation of test compound and terbium chelate-labeled menin for 30 min at ambient temperature, 4 μL 2X FITC-MBM1 peptide (FITC-β-alanine-SARWRFPARPGT-NH₂ (SEQ ID NO: 2)) (“FITC” means fluorescein isothiocyanate) in assay buffer was added, the microtiter plate centrifuged at 1000 rpm for 1 min and the assay mixtures incubated for 15 min at ambient temperature. The relative amount of menin FITC--MBM1 complex present in an assay mixture is determined by measuring the homogenous time-resolved fluorescence (HTRF) of the terbium/FITC donor/acceptor fluorphore pair using an EnVision microplate reader (ex. 337 nm/terbium em. 490 nm/FITC em. 520 nm) at ambient temperature. The degree of fluorescence resonance energy transfer (the HTRF value) is expressed as the ratio of the fluorescence emission intensities of the FITC and terbium fluorophores (F^{em} 520 nm/F^{em} 490 nm). The final concentrations of reagents in the binding assay are 200 pM terbium chelate-labeled menin, 75 nM FITC-MBM1 peptide and 0.5% DMSO in assay buffer. Dose-response titrations of test compounds are conducted using an 11 point, four-fold serial dilution scheme, starting typically at 10 μM.

Compound potencies were determined by first calculating % inhibition at each compound concentration according to equation 1:

$$\% inhibition = ( ( H C - L C ) - ( H T R {F}^{compound} - LC ) ) / ( HC - LC ) ) * 100 \qquad ( Eqn 1 )$$

Where LC and HC are the HTRF values of the assay in the presence or absence of a saturating concentration of a compound that competes with FITC-MBM1 for binding to menin, and HTRF^{compound} is the measured HTRF value in the presence of the test compound. HC and LC HTRF values represent an average of at least 10 replicates per plate. For each test compound, % inhibition values were plotted vs. the logarithm of the test compound concentration, and the IC₅₀ value derived from fitting these data to equation 2:

$$\% inhibition = Bottom + ( Top - Bottom ) / ( 1 + 10 \hat{} ( ( log {IC}_{50} - log [ cmpd ] ) * h ) ) \qquad ( Eqn 2 )$$

Where Bottom and Top are the lower and upper asymptotes of the dose-response curve, respectively, IC₅₀ is the concentration of compound that yields 50% inhibition of signal and h is the Hill coefficient. IC₅₀ values below 0.1 nM in the HTRF assay were reported as 0.1 nM in the Table below (detection limit).

Preparation of Terbium cryptate labeling of Menin: Menin (a.a 1-610-6xhis tag, 2.3 mg/mL in 20 mM Hepes (2-[4-(2-Hydroxyethyl)-1-piperazinyl]ethane sulfonic acid), 80 mM NaCl, 5 mM DTT (Dithiothreitol), pH 7.5) was labeled with terbium cryptate as follows. 200 μg of Menin was buffer exchanged into lx Hepes buffer. 6.67 μM Menin was incubated with 8-fold molar excess NHS (N-hydroxysuccinimide)-terbium cryptate for 40 minutes at room temperature. Half of the labeled protein was purified away from free label by running the reaction over a NAP5 column with elution buffer (0.1M Hepes, pH 7+0.1% BSA (bovine serum albumin)). The other half was eluted with 0.1 M phosphate buffered saline (PBS), pH7. 400 μl of eluent was collected for each, aliquoted and frozen at −80° C. The final concentration of terbium-labeled Menin protein was 115 μg/mL in Hepes buffer and 85 μg/mL in PBS buffer, respectively.

MENIN Protein Sequence (SEQ ID NO: 1):
MGLKAAQKTLFPLRSIDDVVRLFAAELGREEPDLVLLSLVLGFVEHFLAVNRVIPTNVPELT
FQPSPAPDPPGGLTYFPVADLSIIAALYARFTAQIRGAVDLSLYPREGGVSSRELVKKVSDV
IWNSLSRSYFKDRAHIQSLFSFITGTKLDSSGVAFAVVGACQALGLRDVHLALSEDHAWVVF
GPNGEQTAEVTWHGKGNEDRRGQTVNAGVAERSWLYLKGSYMRCDRKMEVAFMVCAINPSID
LHTDSLELLQLQQKLLWLLYDLGHLERYPMALGNLADLEELEPTPGRPDPLTLYHKGIASAK
TYYRDEHIYPYMYLAGYHCRNRNVREALQAWADTATVIQDYNYCREDEEIYKEFFEVANDVI
PNLLKEAASLLEAGEERPGEQSQGTQSQGSALQDPECFAHLLRFYDGICKWEEGSPTPVLHV
GWATFLVQSLGRFEGQVRQKVRIVSREAEAAEAEEPWGEEAREGRRRGPRRESKPEEPPPPK
KPALDKGLGTGQGAVSGPPRKPPGTVAGTARGPEGGSTAQVPAPAASPPPEGPVLTFQSEKM
KGMKELLVATKINSSAIKLQLTAQSQVQMKKQKVSTPSDYTLSFLKRQRKGLHHHHHH

2) Proliferation assay

The anti-proliferative effect of menin/MLL protein/protein interaction inhibitor test compounds was assessed in human leukemia cell lines. The cell line MOLM14 harbors a MLL translocation and expresses the MLL fusion proteins MLL-AF9, respectively, as well as the wildtype protein from the second allele. MLL rearranged cell lines (e.g. MOLM14) exhibit stem cell-like HOXA/MEIS1 gene expression signatures. KO-52 was used as a control cell line containing two MLL (KMT2A) wildtype alleles in order to exclude compounds that display general cytotoxic effects.

MOLM14 cells were cultured in RPMI-1640 (Sigma Aldrich) supplemented with 10% heat-inactivated fetal bovine serum (HyClone), 2 mM L-glutamine (Sigma Aldrich) and 50 pg/ml gentamycin (Gibco). KO-52 cell lines were propagated in alpha-MEM (Sigma Aldrich) supplemented with 20% heat-inactivated fetal bovine serum (HyClone), 2 mM L-glutamine (Sigma Aldrich) and 50 pg/ml gentamycin (Gibco). Cells were kept at 0.3-2.5 million cells per ml during culturing and passage numbers did not exceed 20.

In order to assess the anti-proliferative effects, 200 MOLM14 cells, or 300 KO-52 cells were seeded in 200 μl media per well in 96-well round bottom, ultra-low attachment plates (Costar, catalogue number 7007). Cell seeding numbers were chosen based on growth curves to ensure linear growth throughout the experiment. Test compounds were added at different concentrations and the DMSO content was normalized to 0.3%. Cells were incubated for 8 days at 37° C. and 5% CO₂. Spheroid like growth was measured in real-time by live-cell imaging (IncuCyteZOOM, Essenbio, 4× objective) acquiring images at day 8. Confluence (%) as a measure of spheroid size was determined using an integrated analysis tool.

In order to determine the effect of the test compounds over time, the confluence in each well as a measure of spheroid size, was calculated. Confluence of the highest dose of a reference compound was used as baseline for the LC (Low control) and the confluence of DMSO treated cells was used as 0% cytotoxicity (High Control, HC).

Absolute IC₅₀ values were calculated as percent change in confluence as follows:

LC=Low Control: cells treated with e.g. 1 μM of the cytotoxic agent staurosporin, or e.g. cells treated with a high concentration of an alternative reference compound

$$HC = High Control : Mean confluence ( \% ) ( DMSO treated cells )$$

$$\% Effect = 100 - ( 10 \; 0 * ( Sample - LC ) / ( HC - L C ) )$$

GraphPad Prism (version 7.00) was used to calculate the IC₅₀. Dose-response equation was used for the plot of % Effect vs Log10 compound concentration with a variable slope and fixing the maximum to 100% and the minimum to 0%.

TABLE 3
Biological data

| | HTRF- | spheroid | spheroid |
|---|---|---|---|
| | 30 min | assay_OneTime | assay_OneTime |
| Co. | incubation | MOLM14 | KO-52 |
| No. | IC50(nM) | IC50(μM) | IC50(μM) |
| 1 | 0.041 | 0.05 | 11.92 |
| 2 | 0.376 | 0.02 | 14.57 |
| 3 | 0.054 | 0.005 | |
| 4 | 0.208 | 0.021 | |
| 5 | 0.110 | 0.22 | |
| 6 | 0.092 | 0.1 | |
| 7 | 0.121 | 0.007 | |
| 8 | 0.182 | 0.016 | |
| 9 | 0.055 | 0.19 | |
| 10 | 0.086 | 0.46 | |
| 11 | 0.075 | 0.06 | |
| 12 | 0.044 | 0.029 | |
| 13a | 1.966 | | |
| 13b | 0.049 | 0.021 | |
| 14 | 6.662 | >0.94 | |
| 15 | 0.331 | 0.47 | |
| 16 | 2.966 | | |
| 17 | 79.341 | | |
| 18 | 35.457 | | |
| 19 | 0.079 | 0.032 | |
| 20 | 0.290 | 0.43 | |
| 21 | 13.652 | | |
| 22 | 18.205 | | |
| 23 | 4.569 | | |
| 24 | 35.465 | | |
| 25 | 6.464 | | |

1. A compound of Formula (I) 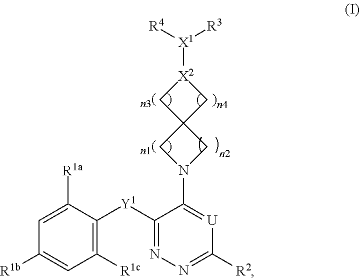
or a tautomer or a stereoisomeric form thereof, wherein
R^{1a} represents —C(═O)—NR^{xa}R^{xb}; Het; or 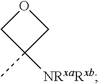
Het represents a 5- or 6-membered monocyclic aromatic ring containing one, two or three nitrogen atoms and optionally a carbonyl moiety;
wherein said 5- or 6-membered monocyclic aromatic ring is optionally substituted with one, two or three substituents selected from the group consisting of C₃₋₆cycloalkyl and C₁₋₄alkyl;
R^{xa} and R^{xb} are each independently selected from the group consisting of hydrogen;
C₁₋₄alkyl; C₃₋₆cycloalkyl; C₁₋₄alkyl substituted with 1, 2 or 3 halo atoms; and C₁₋₄alkyl substituted with one —OH, —OC₁₋₄alkyl, or NR^{11c}R^{11d};
R^{1b} represents F or Cl;
R^{1c} represents H or halo;
Y¹ represents —CR^{5a}R^{5b}—, —O— or —NR^{5c}-;
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents C₁₋₅alkyl; 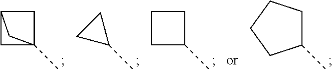
R^{5a}, R^{Sb}R⁵, R^{7a},and R^{7b}, are each independently selected from the group consisting of hydrogen, C₁₋₄alkyl and C₃₋₆cycloalkyl;
R³ represents —C₁₋₆alkyl-NR^{8a}R^{8b}, —C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl;
wherein each of the C₁₋₄alkyl or C₁₋₆alkyl moieties in the R³ definitions independently of each other may be substituted with one, two or three substituents each independently selected from the group consisting of cyano, halo, —OH, and —O—C₁₋₄alkyl;
R^{8a} and R^{8b} are each independently selected from the group consisting of hydrogen;
C₁₋₆alkyl; —C(═O)—C₁₋₄alkyl; —C(═O)—O—C₁₋₄alkyl; —C(═O)—NR^{12a}R^{12b}; and C₁₋₆alkyl substituted with one, two or three substituents each independently selected from the group consisting of —OH, cyano, halo, —C—C, —CH═CH, —S—C₁₋₄alkyl, —S(═O)₂—C₁₋₄alkyl, —S(═O)₂—NR^{11a}R^{11b},
—O—C₁₋₄alkyl, —C(═O)—NR^{10a}R^{10b}, —NR^{10c}—C(═O)—C₁₋₄alkyl, —O—C₁₋₄alkyl substituted with one NR^{11a}R^{11b} and —O—C₁₋₄alkyl substituted with one, two or three halo atoms;
R^{9a}, R^{9b}, R^{10a}, R^{10b}, R^{10c}, R¹¹, R^{11a}, R^{11b}, R^{12a}, and R^{12b} are each independently selected from the group consisting of hydrogen and C₁₋₆alkyl;
R^{11c} and R^{11d} are each independently selected from the group consisting of hydrogen, C₁₋₆alkyl, and —C(═O)—C₁₋₄alkyl;
or a pharmaceutically acceptable salt or a solvate thereof;
provided however that at least one of the following conditions is fulfilled:
a) R^{1a} represents Het wherein the 5- or 6-membered monocyclic aromatic ring is substituted with three substituents selected from the group consisting of C₃₋₆cycloalkyl and C₁₋₄alkyl;
b) R^{1a} represents —C(═O)—NR^{xa}R^{xb}; and R^{xa} is selected from the group consisting of C₁₋₄alkyl substituted with one —OH, —OC₁₋₄alkyl, or NR^{11c}R^{11d}; and C₁₋₄alkyl substituted with 1, 2 or 3 halo atoms;
c) R^{1c} represents halo;
d) R⁴ is other than isopropyl;
e) R³ represents —C₁₋₆alkyl-NR^{8a}R^{1b}; and R^{8a} is C₁₋₆alkyl substituted with one, two or three substituents each independently selected from the group consisting of —C≡C,
—CH═CH, —S—C₁₋₄alkyl, —S(═O)₂—NR^{11a}R^{11b}, —O—C₁₋₄alkyl substituted with one NR^{11a}R^{11b} and —O—C₁₋₄alkyl substituted with one, two or three halo atoms.

2. The compound according to claim 1, wherein
R^{1a} represents —C(═O)—NR^{xa}R^{xb};
R^{xa} and R^{xb} are each independently selected from the group consisting of C₁₋₄alkyl; and
C₁₋₄alkyl substituted with one —OH, or NR^{11c}R^{11d};
R^{1b} represents F;
Y¹ represents —O—;
R² represent hydrogen;
R⁴ represents C₁₋₆alkyl;
R³ represents —C₁₋₆alkyl-NR^{8a}R^{8b};
wherein the C₁₋₆alkyl moiety in the R³ definition may be substituted with one, two or three
—OH substituents;
R^{8a} and R^{8b} are each independently selected from the group consisting of hydrogen;
C₁₋₆alkyl; and C₁₋₆alkyl substituted with one, two or three substituents each independently selected from the group consisting of —C—C, —CH═CH, —S—C₁₋₄alkyl, —S(═O)₂—NR^{11a}R^{11b},
—O—C₁₋₄alkyl, —O—C₁₋₄alkyl substituted with one NR^{11a}R^{11b} and —O—C₁₋₄alkyl substituted with one, two or three halo atoms;
R^{11a} and R^{11b} represent hydrogen;
R^{11c} and R^{11a} are each independently selected from the group consisting of hydrogen and —C(═O)—C₁₋₄alkyl;
provided however that at least one of the following conditions is fulfilled:
a) R^{1a} represents —C(═O)—NR^{xa}R^{xb}; and R^{xa} is selected from the group consisting of C₁₋₄alkyl substituted with one —OH or NR^{11c}R^{11d}
b) R^{11c} represents halo;
c) R⁴ tert-butyl;
d) R³ represents —C₁₋₆alkyl-NR^{8a}R^{8b}; and R^{8a} is C₁₋₆alkyl substituted with one, two or three substituents each independently selected from the group consisting of —C≡C,
—CH═CH, —S—C₁₋₄alkyl, —S(═O)₂—NR^{11a}R^{11b}, —O—C₁₋₄alkyl substituted with one NR^{11a}R^{11b} and —O—C₁₋₄alkyl substituted with one, two or three halo atoms.

3. The compound according to claim 1, wherein R^{1a} represents —C(═O)—NR^{xa}R^{xb}.

4. The compound according to claim 1, wherein
R^{1a} represents —C(═O)—NR^{xa}R^{xb};
Y¹ represents —O—;
U represents N;
n1 is 1, n2 is 2, n3 is 1, and n4 is 1;
R^{1b} represents F;
R^{1c} represents H;
R² represents hydrogen;
R⁴ represents C₁₋₅alkyl; and
R³ represents —C₁₋₆alkyl-NR^{8a}R^{8b}.

5. The compound according to claim 1, wherein U represents N.

6. The compound according to claim 1, wherein Y¹ represents —O—.

7. The compound according to claim 1, wherein Rib represents F.

8. A pharmaceutical composition comprising a compound as claimed in claim 1 and a pharmaceutically acceptable carrier or diluent.

9. A process for preparing a pharmaceutical composition comprising mixing a pharmaceutically acceptable carrier or diluent with a therapeutically effective amount of a compound according to claim 1.

10. A compound as claimed in claim 1 for use as a medicament.

11. A compound as claimed in claim 1 for use in the prevention or treatment of cancer, myelodysplastic syndrome (MDS) and diabetes.

12. The compound for use according to claim 11, wherein cancer is selected from leukemias, myeloma or a solid tumor cancer such as prostate cancer, lung cancer, breast cancer, pancreatic cancer, colon cancer, liver cancer, melanoma and glioblastoma.

13. The compound for use according to claim 12, wherein the leukemia is selected from acute leukemias, chronic leukemias, myeloid leukemias, myelogeneous leukemias, lymphoblastic leukemias, lymphocytic leukemias, Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, and leukemias exhibiting HOX1MEIS1 gene expression signatures.

14. A method of treating or preventing a disorder selected from cancer, myelodysplastic syndrome (MDS) and diabetes comprising administering to a subject in need thereof, a therapeutically effective amount of a compound as claimed in of claim 1.

15. The method according to claim 13 wherein the disorder is cancer.

16. A pharmaceutical composition as claimed in claim 8 for use as a medicament.

17. A pharmaceutical composition as claimed in claim 8 for use in the prevention or treatment of cancer, myelodysplastic syndrome (MDS) and diabetes.

18. The—pharmaceutical composition for use according to claim 17, wherein cancer is selected from leukemias, myeloma or a solid tumor cancer such as prostate cancer, lung cancer, breast cancer, pancreatic cancer, colon cancer, liver cancer, melanoma and glioblastoma.

19. The pharmaceutical composition for use according to claim 18, wherein the leukemia is selected from acute leukemias, chronic leukemias, myeloid leukemias, myelogeneous leukemias, lymphoblastic leukenias, lymphocytic leukenias, Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, and leukemias exhibiting HOX1EIS1 gene expression signatures.

20. A method of treating or preventing a disorder selected from cancer, myelodysplastic syndrome (MDS) and diabetes comprising administering to a subject in need thereof, a therapeutically effective amount of a pharmaceutical composition as claimed in claim 8.

21. The method according to claim 20 wherein the disorder is cancer.


SEQUENCE LISTING (2 sequences)

SEQ ID NO: 1 - protein, 616 aa, Artificial Sequence
  Description of Artificial Sequence Synthetic polypeptide
MGLKAAQKTLFPLRSIDDVVRLFAAELGREEPDLVLLSLVLGFVEHFLAVNRVIPTNVPELTFQPSPAPDPPGGLTYFPVADLSIIAALYARFTAQIRGAVDLSLYPREGGVSSRELVKKVSDVIWNSLSRSYFKDRAHIQSLFSFITGTKLDSSGVAFAVVGACQALGLRDVHLALSEDHAWVVFGPNGEQTAEVTWHGKGNEDRRGQTVNAGVAERSWLYLKGSYMRCDRKMEVAFMVCAINPSIDLHTDSLELLQLQQKLLWLLYDLGHLERYPMALGNLADLEELEPTPGRPDPLTLYHKGIASAKTYYRDEHIYPYMYLAGYHCRNRNVREALQAWADTATVIQDYNYCREDEEIYKEFFEVANDVIPNLLKEAASLLEAGEERPGEQSQGTQSQGSALQDPECFAHLLRFYDGICKWEEGSPTPVLHVGWATFLVQSLGRFEGQVRQKVRIVSREAEAAEAEEPWGEEAREGRRRGPRRESKPEEPPPPKKPALDKGLGTGQGAVSGPPRKPPGTVAGTARGPEGGSTAQVPAPAASPPPEGPVLTFQSEKMKGMKELLVATKINSSAIKLQLTAQSQVQMKKQKVSTPSDYTLSFLKRQRKGLHHHHHH

SEQ ID NO: 2 - protein, 13 aa, Artificial Sequence
  Description of Artificial Sequence Synthetic peptide
  MOD_RES (1)..(1): Beta-alanine
XSARWRFPARPGT